(19) United States
(12) Patent Application Publication
(10) Pub. No.: US 2012/0083845 A1
(43) Pub. Date: Apr. 5, 2012

(54) COMPOUND SPINAL ROD AND METHOD FOR DYNAMIC STABILIZATION OF THE SPINE

(75) Inventors: Charles J. Winslow, Walnut Creek, CA (US); Steven T. Mitchell, Pleasant Hill, CA (US); John J. Flynn, Walnut Creek, CA (US); James F. Zucherman, San Francisco, CA (US); Ken Y. Hsu, San Francisco, CA (US); Henry A. Klyce, Piedmont, CA (US); H. Adam R. Klyce, Berkeley, CA (US)

(73) Assignee: Spartek Medical, Inc., Alameda, CA (US)

(21) Appl. No.: 12/898,139

(22) Filed: Oct. 5, 2010

(51) Int. Cl. A61B 17/70; A61B 17/86

(52) U.S. Cl. 606/264; 606/305

(57) ABSTRACT

Compound spinal rods function as part of the dynamic stabilization prosthesis to provide load sharing with motion preservation as part of a dynamic stabilization prosthesis. Compound spinal rods are used to span from one vertebra to an adjacent vertebra. Compound spinal rods include a first rod connected by a linkage to a second rod. The linkage allows for movement of the first rod relative to the second rod. The movement permitted by the compound spinal rod is designed to enhance the ability of a dynamic stabilization prosthesis to more closely approximate the natural kinematics of the spine without impairing stabilization of the spine.

### CLAIM TO PRIORITY

This application claims priority to the following patents and patent applications:

U.S. patent application Ser. No. 12/566,485, filed Sep. 24, 2009, entitled “VERSATILE POLYAXIAL CONNECTOR ASSEMBLY AND METHOD FOR DYNAMIC STABILIZATION OF THE SPINE” (Attorney Docket No. SPART-01043US1) which claims priority to U.S. Provisional 61/100,625, filed Sep. 26, 2008, entitled “VERSALTILE ASSEMBLY COMPONENTS AND METHODS FOR A DYNAMIC SPINAL STABILIZATION” (Attorney Docket No. SPART-01043US0); and

U.S. patent application Ser. No. 12/566,487, filed Sep. 24, 2009, entitled “VERSATILE OFFSET POLYAXIAL CONNECTOR AND METHOD FOR DYNAMIC STABILIZATION OF THE SPINE” (Attorney Docket No. SPART-01043US2) which claims priority to U.S. Provisional 61/100,625, filed Sep. 26, 2008, entitled “VERSATILE ASSEMBLY COMPONENTS AND METHODS FOR A DYNAMIC SPINAL STABILIZATION” (Attorney Docket No. SPART-01043US0); and

U.S. patent application Ser. No. 12/566,491, filed Sep. 24, 2009, entitled “LOAD-SHARING BONE ANCHOR HAVING A DEFLECTABLE POST AND METHODS FOR DYNAMIC STABILIZATION OF THE SPINE” (Attorney Docket No. SPART-01044US1) which claims priority to U.S. Provisional 61/119,651, filed Dec. 3, 2008, entitled “LOAD-SHARING COMPONENT HAVING A DEFLECTABLE POST AND METHODS FOR DYNAMIC SPINAL STABILIZATION” (Attorney Docket No. SPART-01044US0), and which claims priority to U.S. Provisional 61/122,658, filed Dec. 15, 2008, entitled “LOAD-SHARING COMPONENT HAVING A DEFLECTABLE POST AND METHODS FOR DYNAMIC SPINAL STABILIZATION” (Attorney Docket No. SPART-01044US2), and which claims priority to U.S. Provisional 61/144,426, filed Jan. 13, 2009, entitled “LOAD-SHARING COMPONENT HAVING A DEFLECTABLE POST AND METHODS FOR DYNAMIC SPINAL STABILIZATION” (Attorney Docket No. SPART-01044US3), and which claims priority to U.S. Provisional 61/225,478, filed Jul. 14, 2009, entitled “LOAD-SHARING COMPONENT HAVING A DEFLECTABLE POST AND METHODS FOR DYNAMIC SPINAL STABILIZATION” (Attorney Docket No. SPART-01044US4); and

U.S. patent application Ser. No. 12/566,494, filed Sep. 24, 2009, entitled “LOAD-SHARING COMPONENT HAVING A DEFLECTABLE POST AND METHOD FOR DYNAMIC STABILIZATION OF THE SPINE” (Attorney Docket No. SPART-01044US5) which claims priority to U.S. Provisional 61/119,651, filed Dec. 3, 2008, entitled “LOAD-SHARING COMPONENT HAVING A DEFLECTABLE POST AND METHODS FOR DYNAMIC SPINAL STABILIZATION” (Attorney Docket No. SPART-01044US0), and which claims priority to U.S. Provisional 61/122,658, filed Dec. 15, 2008, entitled “LOAD-SHARING COMPONENT HAVING A DEFLECTABLE POST AND METHODS FOR DYNAMIC SPINAL STABILIZATION” (Attorney Docket No. SPART-01044US2), and which claims priority to U.S. Provisional 61/144,426, filed Jan. 13, 2009, entitled “LOAD-SHARING COMPONENT HAVING A DEFLECTABLE POST AND METHODS FOR DYNAMIC SPINAL STABILIZATION” (Attorney Docket No. SPART-01044US3), and which claims priority to U.S. Provisional 61/225,478, filed Jul. 14, 2009, entitled “LOAD-SHARING COMPONENT HAVING A DEFLECTABLE POST AND METHODS FOR DYNAMIC SPINAL STABILIZATION” (Attorney Docket No. SPART-01044US4); and

U.S. patent application Ser. No. 12/566,498, filed Sep. 24, 2009, entitled “LOAD-SHARING BONE ANCHOR HAVING A DURABLE COMPLIANT MEMBER AND METHOD FOR DYNAMIC STABILIZATION OF THE SPINE” (Attorney Docket No. SPART-01044US6) which claims priority to U.S. Provisional 61/119,651, filed Dec. 3, 2008, entitled “LOAD-SHARING COMPONENT HAVING A DEFLECTABLE POST AND METHODS FOR DYNAMIC SPINAL STABILIZATION” (Attorney Docket No. SPART-01044US0), and which claims priority to U.S. Provisional 61/122,658, filed Dec. 15, 2008, entitled “LOAD-SHARING COMPONENT HAVING A DEFLECTABLE POST AND METHODS FOR DYNAMIC SPINAL STABILIZATION” (Attorney Docket No. SPART-01044US2), and which claims priority to U.S. Provisional 61/144,426, filed Jan. 13, 2009, entitled “LOAD-SHARING COMPONENT HAVING A DEFLECTABLE POST AND METHODS FOR DYNAMIC SPINAL STABILIZATION” (Attorney Docket No. SPART-01044US3), and which claims priority to U.S. Provisional 61/225,478, filed Jul. 14, 2009, entitled “LOAD-SHARING COMPONENT HAVING A DEFLECTABLE POST AND METHODS FOR DYNAMIC SPINAL STABILIZATION” (Attorney Docket No. SPART-01044US4); and

U.S. patent application Ser. No. 12/566,504, filed Sep. 24, 2009, entitled “LOAD-SHARING BONE ANCHOR HAVING A DEFLECTABLE POST WITH A COMPLIANT RING AND METHOD FOR STABILIZATION OF THE SPINE” (Attorney Docket No. SPART-01044US7) which claims priority to U.S. Provisional 61/119,651, filed Dec. 3, 2008, entitled “LOAD-SHARING COMPONENT HAVING A DEFLECTABLE POST AND METHODS FOR DYNAMIC SPINAL STABILIZATION” (Attorney Docket No. SPART-01044US0), and which claims priority to U.S. Provisional 61/122,658, filed Dec. 15, 2008, entitled “LOAD-SHARING COMPONENT HAVING A DEFLECTABLE POST AND METHODS FOR DYNAMIC SPINAL STABILIZATION” (Attorney Docket No. SPART-01044US2), and which claims priority to U.S. Provisional 61/144,426, filed Jan. 13, 2009, entitled “LOAD-SHARING COMPONENT HAVING A DEFLECTABLE POST AND METHODS FOR DYNAMIC SPINAL STABILIZATION” (Attorney Docket No. SPART-01044US3), and which claims priority to U.S. Provisional 61/225,478, filed Jul. 14, 2009, entitled “LOAD-SHARING COMPONENT HAVING A DEFLECTABLE POST AND METHODS FOR DYNAMIC SPINAL STABILIZATION” (Attorney Docket No. SPART-01044US4); and

U.S. patent application Ser. No. 12/566,507, filed Sep. 24, 2009, entitled “LOAD-SHARING BONE ANCHOR HAVING A DEFLECTABLE POST WITH A COMPLIANT RING AND METHOD FOR STABILIZATION OF THE SPINE” (Attorney Docket No. SPART-01044US8) which claims priority to U.S. Provisional 61/119,651, filed Dec. 3, 2008, entitled “LOAD-SHARING COMPONENT HAVING A DEFLECTABLE POST AND METHODS FOR DYNAMIC SPINAL STABILIZATION” (Attorney Docket No. SPART-01044US0), and which claims priority to U.S. Provisional 61/122,658, filed Dec. 15, 2008, entitled “LOAD-SHARING COMPONENT HAVING A DEFLECTABLE POST AND METHODS FOR DYNAMIC SPINAL STABILIZATION” (Attorney Docket No. SPART-01044US2), and which claims priority to U.S. Provisional 61/144,426, filed Jan. 13, 2009, entitled “LOAD-SHARING COMPONENT HAVING A DEFLECTABLE POST AND METHODS FOR DYNAMIC SPINAL STABILIZATION” (Attorney Docket No. SPART-01044US3), and which claims priority to U.S. Provisional 61/225,478, filed Jul. 14, 2009, entitled “LOAD-SHARING COMPONENT HAVING A DEFLECTABLE POST AND METHODS FOR DYNAMIC SPINAL STABILIZATION” (Attorney Docket No. SPART-01044US4); and

U.S. patent application Ser. No. 12/566,511, filed Sep. 24, 2009, entitled “LOAD-SHARING BONE ANCHOR HAVING A DEFLECTABLE POST AND METHOD FOR STABILIZATION OF THE SPINE” (Attorney Docket No. SPART-01044US9) which claims priority to U.S. Provisional 61/119,651, filed Dec. 3, 2008, entitled “LOAD-SHARING COMPONENT HAVING A DEFLECTABLE POST AND METHODS FOR DYNAMIC SPINAL STABILIZATION” (Attorney Docket No. SPART-01044US0), and which claims priority to U.S. Provisional 61/122,658, filed Dec. 15, 2008, entitled “LOAD-SHARING COMPONENT HAVING A DEFLECTABLE POST AND METHODS FOR DYNAMIC SPINAL STABILIZATION” (Attorney Docket No. SPART-01044US2), and which claims priority to U.S. Provisional 61/144,426, filed Jan. 13, 2009, entitled “LOAD-SHARING COMPONENT HAVING A DEFLECTABLE POST AND METHODS FOR DYNAMIC SPINAL STABILIZATION” (Attorney Docket No. SPART-01044US3), and which claims priority to U.S. Provisional 61/225,478, filed Jul. 14, 2009, entitled “LOAD-SHARING COMPONENT HAVING A DEFLECTABLE POST AND METHODS FOR DYNAMIC SPINAL STABILIZATION” (Attorney Docket No. SPART-01044US4); and

U.S. patent application Ser. No. 12/566,516, filed Sep. 24, 2009, entitled “LOAD-SHARING BONE ANCHOR HAVING A NATURAL CENTER OF ROTATION AND METHOD FOR DYNAMIC STABILIZATION OF THE SPINE” (Attorney Docket No. SPART-01044USA) which claims priority to U.S. Provisional 61/119,651, filed Dec. 3, 2008, entitled “LOAD-SHARING COMPONENT HAVING A DEFLECTABLE POST AND METHODS FOR DYNAMIC SPINAL STABILIZATION” (Attorney Docket No. SPART-01044US0), and which claims priority to U.S. Provisional 61/122,658, filed Dec. 15, 2008, entitled “LOAD-SHARING COMPONENT HAVING A DEFLECTABLE POST AND METHODS FOR DYNAMIC SPINAL STABILIZATION” (Attorney Docket No. SPART-01044US2), and which claims priority to U.S. Provisional 61/144,426, filed Jan. 13, 2009, entitled “LOAD-SHARING COMPONENT HAVING A DEFLECTABLE POST AND METHODS FOR DYNAMIC SPINAL STABILIZATION” (Attorney Docket No. SPART-01044US3), and which claims priority to U.S. Provisional 61/225,478, filed Jul. 14, 2009, entitled “LOAD-SHARING COMPONENT HAVING A DEFLECTABLE POST AND METHODS FOR DYNAMIC SPINAL STABILIZATION” (Attorney Docket No. SPART-01044US4); and

U.S. patent application Ser. No. 12/566,519, filed Sep. 24, 2009, entitled “DYNAMIC SPINAL ROD AND METHOD FOR DYNAMIC STABILIZATION OF THE SPINE” (Attorney Docket No. SPART-01044USC) which claims priority to U.S. Provisional 61/119,651, filed Dec. 3, 2008, entitled “LOAD-SHARING COMPONENT HAVING A DEFLECTABLE POST AND METHODS FOR DYNAMIC SPINAL STABILIZATION” (Attorney Docket No. SPART-01044US0), and which claims priority to U.S. Provisional 61/122,658, filed Dec. 15, 2008, entitled “LOAD-SHARING COMPONENT HAVING A DEFLECTABLE POST AND METHODS FOR DYNAMIC SPINAL STABILIZATION” (Attorney Docket No. SPART-01044US2), and which claims priority to U.S. Provisional 61/144,426, filed Jan. 13, 2009, entitled “LOAD-SHARING COMPONENT HAVING A DEFLECTABLE POST AND METHODS FOR DYNAMIC SPINAL STABILIZATION” (Attorney Docket No. SPART-01044US3), and which claims priority to U.S. Provisional 61/225,478, filed Jul. 14, 2009, entitled “LOAD-SHARING COMPONENT HAVING A DEFLECTABLE POST AND METHODS FOR DYNAMIC SPINAL STABILIZATION” (Attorney Docket No. SPART-01044US4); and

U.S. patent application Ser. No. 12/566,522, filed Sep. 24, 2009, entitled “DYNAMIC SPINAL ROD ASSEMBLY AND METHOD FOR DYNAMIC STABILIZATION OF THE SPINE” (Attorney Docket No. SPART-01044USD) which claims priority to U.S. Provisional 61/119,651, filed Dec. 3, 2008, entitled “LOAD-SHARING COMPONENT HAVING A DEFLECTABLE POST AND METHODS FOR DYNAMIC SPINAL STABILIZATION” (Attorney Docket No. SPART-01044US0), and which claims priority to U.S. Provisional 61/122,658, filed Dec. 15, 2008, entitled “LOAD-SHARING COMPONENT HAVING A DEFLECTABLE POST AND METHODS FOR DYNAMIC SPINAL STABILIZATION” (Attorney Docket No. SPART-01044US2), and which claims priority to U.S. Provisional 61/144,426, filed Jan. 13, 2009, entitled “LOAD-SHARING COMPONENT HAVING A DEFLECTABLE POST AND METHODS FOR DYNAMIC SPINAL STABILIZATION” (Attorney Docket No. SPART-01044US3), and which claims priority to U.S. Provisional 61/225,478, filed Jul. 14, 2009, entitled “LOAD-SHARING COMPONENT HAVING A DEFLECTABLE POST AND METHODS FOR DYNAMIC SPINAL STABILIZATION” (Attorney Docket No. SPART-01044US4); and

U.S. patent application Ser. No. 12/566,529, filed Sep. 24, 2009, entitled “CONFIGURABLE DYNAMIC SPINAL ROD AND METHOD FOR DYNAMIC STABILIZATION OF THE SPINE” (Attorney Docket No. SPART-01044USE) which claims priority to U.S. Provisional 61/119,651, filed Dec. 3, 2008, entitled “LOAD-SHARING COMPONENT HAVING A DEFLECTABLE POST AND METHODS FOR DYNAMIC SPINAL STABILIZATION” (Attorney Docket No. SPART-01044US0), and which claims priority to U.S. Provisional 61/122,658, filed Dec. 15, 2008, entitled “LOAD-SHARING COMPONENT HAVING A DEFLECTABLE POST AND METHODS FOR DYNAMIC SPINAL STABILIZATION” (Attorney Docket No. SPART-01044US2), and which claims priority to U.S. Provisional 61/144,426, filed Jan. 13, 2009, entitled “LOAD-SHARING COMPONENT HAVING A DEFLECTABLE POST AND METHODS FOR DYNAMIC SPINAL STABILIZATION” (Attorney Docket No. SPART-01044US3), and which claims priority to U.S. Provisional 61/225,478, filed Jul. 14, 2009, entitled “LOAD-SHARING COMPONENT HAVING A DEFLECTABLE POST AND METHODS FOR DYNAMIC SPINAL STABILIZATION” (Attorney Docket No. SPART-01044US4); and

U.S. patent application Ser. No. 12/566,531, filed Sep. 24, 2009, entitled “A SPINAL PROSTHESIS HAVING A THREE BAR LINKAGE FOR MOTION PRESERVATION AND DYNAMIC STABILIZATION OF THE SPINE” (Attorney Docket No. SPART-01044USF) which claims priority to U.S. Provisional 61/119,651, filed Dec. 3, 2008, entitled “LOAD-SHARING COMPONENT HAVING A DEFLECTABLE POST AND METHODS FOR DYNAMIC SPINAL STABILIZATION” (Attorney Docket No. SPART-01044US0), and which claims priority to U.S. Provisional 61/122,658, filed Dec. 15, 2008, entitled “LOAD-SHARING COMPONENT HAVING A DEFLECTABLE POST AND METHODS FOR DYNAMIC SPINAL STABILIZATION” (Attorney Docket No. SPART-01044US2), and which claims priority to U.S. Provisional 61/144,426, filed Jan. 13, 2009, entitled “LOAD-SHARING COMPONENT HAVING A DEFLECTABLE POST AND METHODS FOR DYNAMIC SPINAL STABILIZATION” (Attorney Docket No. SPART-01044US3), and which claims priority to U.S. Provisional 61/225,478, filed Jul. 14, 2009, entitled “LOAD-SHARING COMPONENT HAVING A DEFLECTABLE POST AND METHODS FOR DYNAMIC SPINAL STABILIZATION” (Attorney Docket No. SPART-01044US4); and

U.S. patent application Ser. No. 12/566,551, filed Sep. 24, 2009, entitled “LOAD-SHARING BONE ANCHOR HAVING A DEFLECTABLE POST AND CENTERING SPRING AND METHOD FOR DYNAMIC STABILIZATION OF THE SPINE” (Attorney Docket No. SPART-01049US1) which claims priority to U.S. Provisional 61/167,789, filed Apr. 8, 2009, entitled “LOAD-SHARING COMPONENT HAVING A DEFLECTABLE POST AND SPRING METHODS FOR DYNAMIC SPINAL STABILIZATION” (Attorney Docket No. SPART-01049US0); and

U.S. patent application Ser. No. 12/566,553, filed Sep. 24, 2009, entitled “LOAD-SHARING COMPONENT HAVING A DEFLECTABLE POST AND CENTERING SPRING AND METHOD FOR DYNAMIC STABILIZATION OF THE SPINE” (Attorney Docket No. SPART-01049US2) which claims priority to U.S. Provisional 61/167,789, filed Apr. 8, 2009, entitled “LOAD-SHARING COMPONENT HAVING A DEFLECTABLE POST AND SPRING METHODS FOR DYNAMIC SPINAL STABILIZATION” (Attorney Docket No. SPART-01049US0); and

U.S. Provisional Application No. 61/261,545, filed Nov. 16, 2009, entitled “LOAD-SHARING BONE ANCHOR HAVING A FLEXIBLE POST AND METHOD FOR DYNAMIC STABILIZATION OF THE SPINE” (Attorney Docket No. SPART-01050US0); and

U.S. patent application Ser. No. 12/566,559, filed Sep. 24, 2009, entitled “LOAD-SHARING BONE ANCHOR HAVING A DEFLECTABLE POST AND AXIAL SPRING AND METHOD FOR DYNAMIC STABILIZATION OF THE SPINE” (Attorney Docket No. SPART-01053US1) which claims priority to U.S. Provisional 61/217,556, filed Jun. 1, 2009, entitled “LOAD-SHARING COMPONENT HAVING A DEFLECTABLE POST AND SPRING METHODS FOR DYNAMIC SPINAL STABILIZATION” (Attorney Docket No. SPART-01053US0); and

U.S. patent application Ser. No. 12/629,811, filed Dec. 2, 2009, entitled “LOW PROFILE SPINAL PROSTHESIS INCORPORATING A BONE ANCHOR HAVING A DEFLECTABLE POST AND COMPOUND SPINAL ROD” (Attorney Docket No. SPART-01057US1) which claims priority to U.S. Provisional 61/119,651, filed Dec. 3, 2008, entitled “LOAD-SHARING COMPONENT HAVING A DEFLECTABLE POST AND METHODS FOR DYNAMIC SPINAL STABILIZATION (Attorney Docket No. SPART-01044US0) and which claims priority to U.S. Provisional 61/122,658, filed Dec. 15, 2008, entitled “LOAD-SHARING COMPONENT HAVING A DEFLECTABLE POST AND METHODS FOR DYNAMIC SPINAL STABILIZATION”(Attorney Docket No. SPART-01044US2) and which claims priority to U.S. Provisional 61/144,426, filed Jan. 13, 2009, entitled “LOAD-SHARING COMPONENT HAVING A DEFLECTABLE POST AND METHODS FOR DYNAMIC SPINAL STABILIZATION” (Attorney Docket No. SPART-01044US3) and which claims priority to U.S. Provisional 61/225,478, filed Jul. 14, 2009, entitled “LOAD-SHARING COMPONENT HAVING A DEFLECTABLE POST AND METHODS FOR DYNAMIC SPINAL STABILIZATION”(Attorney Docket No. SPART-01044US4).

All of the afore-mentioned patent applications are incorporated herein by reference in their entireties.

### BACKGROUND OF INVENTION

Back pain is a significant clinical problem and the costs to treat it, both surgical and medical, is estimated to be over $2 billion per year. One method for treating a broad range of degenerative spinal disorders is spinal fusion. Implantable medical devices designed to fuse vertebrae of the spine to treat have developed rapidly over the last decade. However, spinal fusion has several disadvantages including reduced range of motion and accelerated degenerative changes adjacent the fused vertebrae.

Alternative devices and treatments have been developed for treating degenerative spinal disorders while preserving motion. These devices and treatments offer the possibility of treating degenerative spinal disorders without the disadvantages of spinal fusion. However, current devices and treatments suffer from disadvantages e.g., complicated implantation procedures; lack of flexibility to conform to diverse patient anatomy; the need to remove tissue and bone for implantation; increased stress on spinal anatomy; insecure anchor systems; poor durability, and poor revision options. Consequently, there is a need for new and improved devices and methods for treating degenerative spinal disorders while preserving motion.

### SUMMARY OF INVENTION

The present invention includes a versatile spinal implant system and methods that can dynamically stabilize the spine while providing for the preservation of spinal motion. Embodiments of the invention provide a dynamic stabilization system which includes: versatile components, adaptable stabilization assemblies, and methods of implantation. An aspect of the invention is restoring and/or preserving the natural motion of the spine including the quality of motion as well as the range of motion. Another aspect of the invention is providing for load sharing and stabilization of the spine while preserving motion. Still another aspect of the invention is the ability to stabilize two, three and/or more levels of the spine. Another aspect of the invention is the versatility of assembly of a spinal stabilization prosthesis utilizing the components to accommodate the functional requirements and anatomy of the patient. Another aspect of the invention is to provide a range of components which allows selection of components appropriate to the application and patient anatomy. Another aspect of the invention is to provide components which stabilize the spine while reducing stresses placed on individual components and the interface between those components and the bone of the spine. Another aspect of the invention is to provide components which isolate components of the spinal stabilization assembly which mount to the bone from stresses and loads placed on other components of the spinal stabilization assembly. Another aspect of the invention is to provide procedures and devices which facilitate the process of implantation and assembly. Another aspect of the invention is to provide procedures and devices which minimize disruption of tissues during implantation of a spinal stabilization assembly. Thus, the present invention provides new and improved systems, devices and methods for treating spinal disorders.

A particular aspect of the invention is to provide a spinal rod which provides load sharing with motion preservation as part of a dynamic stabilization prosthesis. Another aspect of the invention is to provide compound spinal rods which include a first rod connected by a linkage to a second rod. Another aspect of the invention is to provide a compound spinal rod which enhances the ability of a dynamic stabilization prosthesis to approximate the natural kinematics of the spine without impairing stabilization of the spine.

These and other objects, features and advantages of the invention will be apparent from the drawings and detailed description which follow.

### BRIEF DESCRIPTION OF THE DRAWINGS

FIG. 1A is a perspective view of a deflection rod assembled with a bone anchor according to an embodiment of the present invention.

FIG. 1B is a perspective view of an offset connector mounted to the bone anchor of FIG. 1A.

FIG. 1C is a perspective view of a compound spinal rod mounted to the bone anchor of FIG. 1A according to an embodiment of the present invention.

FIG. 1D is a posterior view of a multi-level dynamic stabilization prosthesis utilizing the components of FIGS. 1A to 1C according to an embodiment of the present invention.

FIG. 1E is a lateral view of the multi-level dynamic stabilization prosthesis of FIG. 1D.

FIG. 2A is an exploded view of bone anchor according to an embodiment of the invention.

FIG. 2B is a perspective view of the bone anchor of FIG. 2A.

FIGS. 2C and 2D are sectional views of the bone anchor of FIG. 2A.

FIG. 2E is a perspective view of the bone anchor of FIG. 2A in combination the connector of FIG. 1B and compound spinal rod of FIG. 1C.

FIGS. 3A, 3B, and 3C are exploded, sectional, and perspective views of a compound spinal rod according to an embodiment of the present invention.

FIG. 4A is a lateral view of the lumbar spine illustrating the natural kinematics of the spine during extension and flexion.

FIG. 4B is a lateral view of the lumbar spine illustrating the kinematic constraints placed on the spine by a rigid spinal rod system during extension and flexion.

FIGS. 4C and 4D show the kinematic modes of an embodiment of the dynamic spine stabilization prosthesis of the invention utilizing a bone anchor and a compound spinal rod in accordance with embodiments of the invention.

FIG. 4E is a graph illustrating the kinematics of the dynamic spine stabilization prosthesis of FIGS. 4C and 4D.

FIG. 4F is a lateral view of the spine illustrating the kinematics of the spine supported by the dynamic spine stabilization prosthesis of FIGS. 4C, 4D, and 4E.

FIGS. 5A, 5B and 5C are exploded, sectional and perspective views of an alternative compound spinal rod and its components in accordance with an embodiment of the present invention.

FIG. 5D shows the kinematic modes of the compound spinal rod of FIGS. 5A, 5B and 5C.

FIG. 5E shows a lateral view of a dynamic spine stabilization prosthesis incorporating the compound spinal rod of FIGS. 5A-5C in accordance with an embodiment of the present invention.

FIGS. 6A and 6B are exploded and perspective views of an alternative compound spinal rod and its components in accordance with an embodiment of the present invention.

FIG. 6C shows the kinematic modes of the compound spinal rod of FIGS. 6A and 6B.

FIG. 6D shows a lateral view of a dynamic spine stabilization prosthesis incorporating the compound spinal rod of FIGS. 6A-6B in accordance with an embodiment of the present invention.

FIGS. 7A, 7B and 7C are exploded, sectional, and perspective views of an alternative compound spinal rod and its components in accordance with an embodiment of the present invention.

FIGS. 8A, 8B and 8C are exploded, sectional, and perspective views of an alternative compound spinal rod and its components in accordance with an embodiment of the present invention.

FIGS. 9A, 9B and 9C are exploded, perspective, and sectional views of a coupling adapted to connect a rod to a post or deflectable post in accordance with an embodiment of the present invention.

FIGS. 10A, 10B and 10C are exploded, sectional, and perspective views of an alternative compound spinal rod according to an embodiment of the present invention.

FIGS. 10D-10G show views of alternative compliant members for the compound spinal rod of FIGS. 10A-10C.

FIGS. 11A, 11B and 11C are exploded, sectional, and perspective views of an alternative compound spinal rod according to an embodiment of the present invention.

FIG. 11D shows an enlarged perspective views of the compliant member of the compound spinal rod of FIGS. 10A-10C.

FIGS. 11E-11H show views of alternative compliant members for the compound spinal rod of FIGS. 11A-11C.

FIG. 12A is a perspective view of an alternative compound spinal rod according to an embodiment of the present invention.

FIGS. 12B and 12C are enlarged views of components of the compound spinal rod of FIG. 12A.

FIGS. 12D and 12E are sectional views of the compound spinal rod of FIG. 12A illustrating deflection or the compound spinal rod.

FIGS. 13A, 13B and 13C are exploded, sectional, and perspective views of an alternative compound spinal rod according to an embodiment of the present invention.

FIGS. 14A, 14B and 14C are exploded, sectional, and perspective views of an alternative compound spinal rod according to an embodiment of the present invention.

FIG. 14D is a perspective view of a variation of the compound spinal rod of FIGS. 14A-14C according to an embodiment of the present invention.

### DETAILED DESCRIPTION OF THE PREFERRED EMBODIMENTS

The present invention includes a versatile spinal stabilization system and methods which can dynamically stabilize the spine while providing for the preservation of spinal motion. Alternative embodiments can be used for spinal fusion. In one embodiment the invention provides a system for restoring and/or preserving the natural motion of the spine including the quality of motion as well as the range of motion. In another embodiment the invention provides load sharing and stabilization of the spine while preserving motion. In another embodiment the invention provides the ability to stabilize two, three and/or more levels of the spine. In another embodiment the invention provides versatile assembly of a spinal stabilization prosthesis utilizing the components to accommodate the functional requirements and anatomy of the patient. In another embodiment the invention provides a range of components which allows selection of components appropriate to the application and patient anatomy. In another embodiment the invention provides components which stabilize the spine while reducing stresses placed on individual components and the interface between those components and the bone of the spine. In another embodiment the invention provides components which isolate other components of the spinal stabilization assembly which mount to the bone from stresses and loads placed on other components of the spinal stabilization assembly. In another embodiment, the invention provides procedures and devices which facilitate the process of implantation and assembly. In another embodiment, the invention provides procedures and devices which minimize disruption of tissues during implantation of a spinal stabilization assembly.

In a particular embodiment, the invention provides a spinal rod which provides load sharing with motion preservation as part of a dynamic stabilization prosthesis. In another particular embodiment, the invention provides compound spinal rods which include a first rod connected by a linkage to a second rod. In another particular embodiment the invention provides a compound spinal rod which enhances the ability of a dynamic stabilization prosthesis to approximate the natural kinematics of the spine without impairing stabilization of the spine.

Embodiments of the present invention provide for assembly of a dynamic stabilization prosthesis which supports the spine while providing for the preservation of spinal motion. The dynamic stabilization system includes an anchor system, a deflection system, a vertical rod system and a connection system. The anchor system anchors the construct to the spinal anatomy. The deflection system provides dynamic stabilization while reducing the stress exerted upon the bone anchors and spinal anatomy. The vertical rod system connects different levels of the construct in a multilevel assembly and may in some embodiments include compound spinal rods. The connection system includes coaxial connectors and offset connectors which adjustably connect the deflection system, vertical rod system and anchor system allowing for appropriate, efficient and convenient placement of the anchor system relative to the spine. Alternative embodiments can be used for spinal fusion.

Compound spinal rods, according to particular embodiments of the present invention, provide load sharing while preserving range of motion and reducing stress exerted upon the bone anchors and spinal anatomy. The compound spinal rod includes a first rod connected to a second rod by a linkage. The linkage allows controlled and/or constrained movement of one rod with respect to the other rod. The linkage may include one or more compliant members and/or limit surfaces to control and/or constrain the movement of one rod with respect to the other rod. The force-deflection properties of the compound spinal rod are adaptable and/or customizable to the anatomy and functional requirements of the patient by changing the properties of the compliant member. Different compound spinal rods having different force-deflection properties are adapted to be utilized in different patients or at different spinal levels within the same patient depending upon the anatomy and functional requirements.

Common reference numerals are used to indicate like elements throughout the drawings and detailed description; therefore, reference numerals used in a drawing may or may not be referenced in the detailed description specific to such drawing if the associated element is described elsewhere. The first digit in a reference numeral indicates the series of figures in which the referenced item first appears.

The terms “vertical” and “horizontal” are used throughout the detailed description to describe general orientation of structures relative to the spine of a human patient that is standing. This application also uses the terms proximal and distal in the conventional manner when describing the components of the spinal implant system. Thus, proximal refers to the end or side of a device or component closest to the hand operating the device, whereas distal refers to the end or side of a device furthest from the hand operating the device. For example, the tip of a screw that enters a bone would conventionally be called the distal end (it is furthest from the surgeon) while the head of the screw would be termed the proximal end (it is closest to the surgeon).

### Dynamic Stabilization System

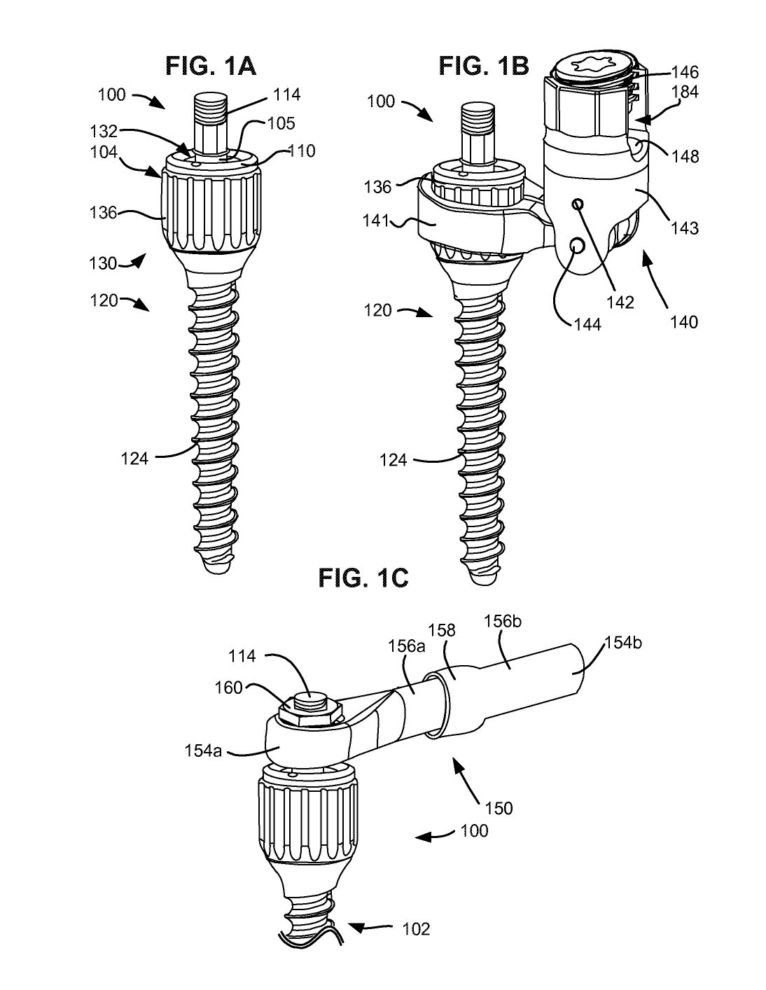

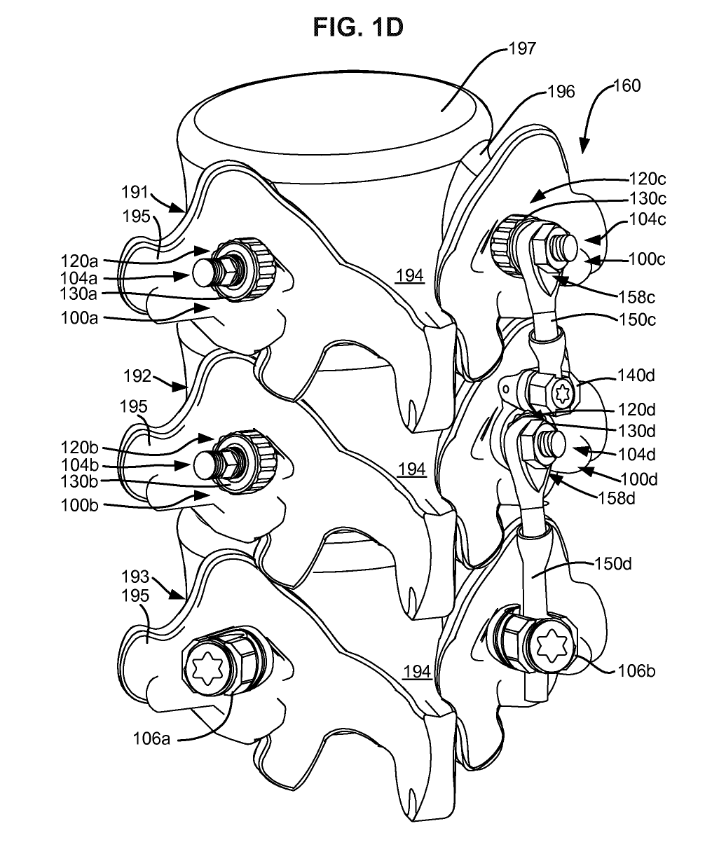

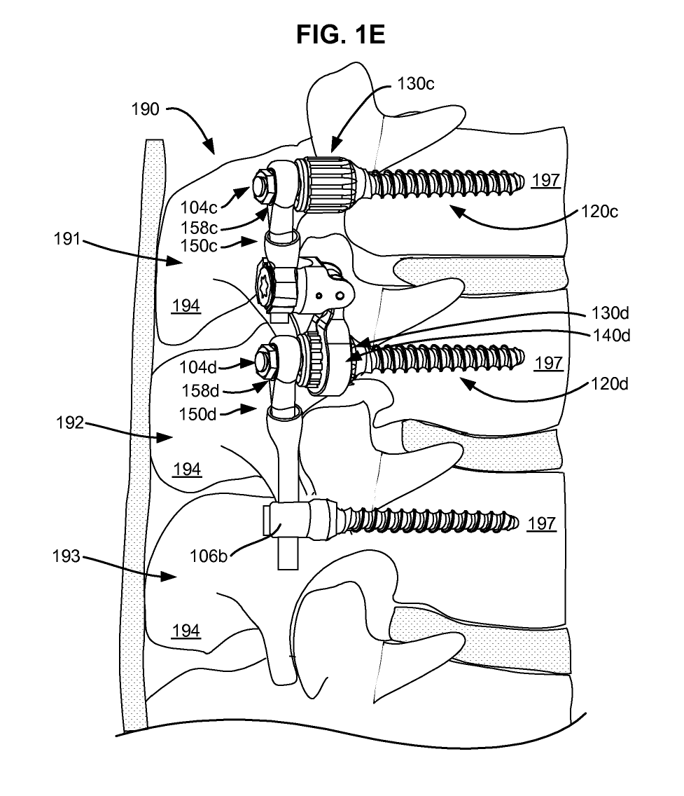

FIGS. 1A-1F introduce components and assemblies of a dynamic stabilization system according to an embodiment of the present invention. The components include anchor system components, deflection rods, vertical rods and connection system components, including for example coaxial and offset connectors. In particular the dynamic stabilization system includes a compound spinal rod. The components are adapted to be implanted and assembled to form a dynamic stabilization prosthesis appropriate for the anatomical and functional needs of a patient.

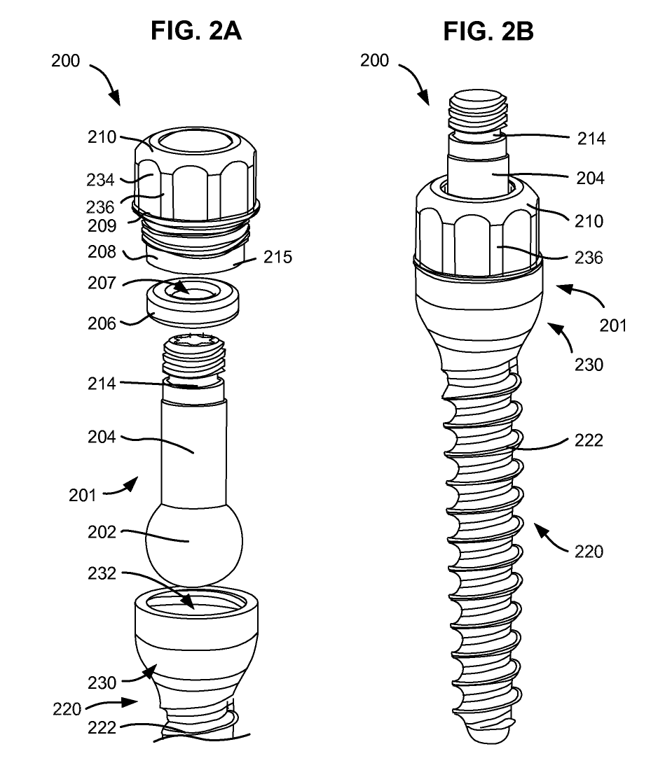

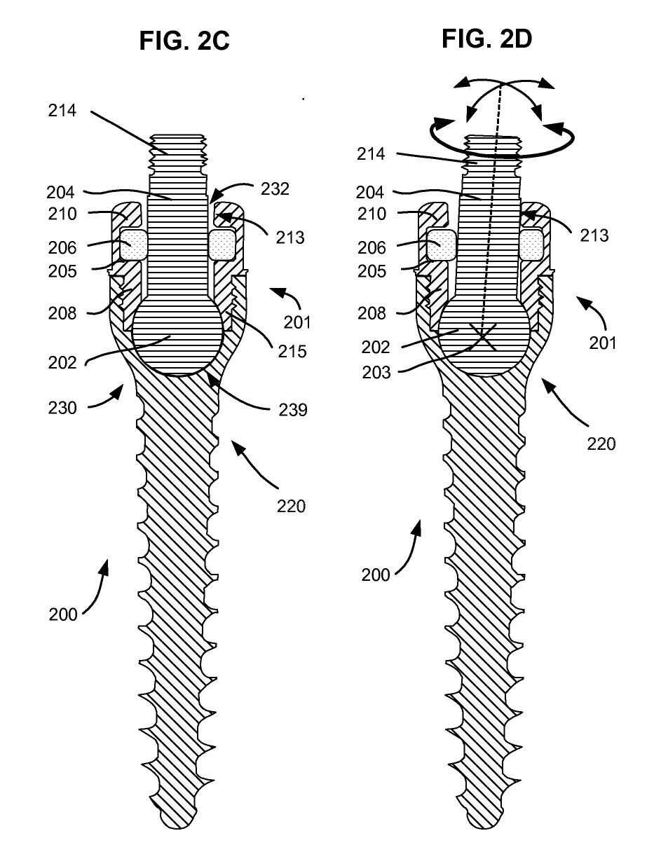

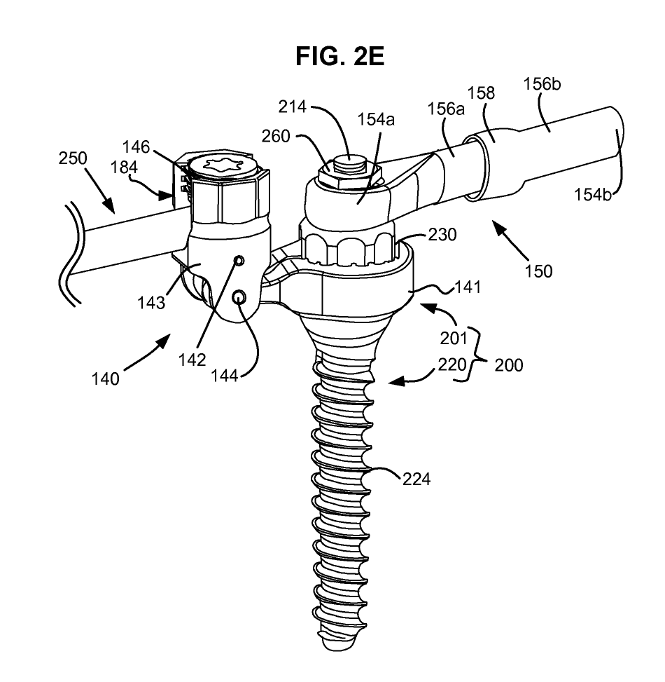

FIG. 1A shows a bone anchor 100 which includes a combination of a deflection rod 104 and bone screw 120. Deflection rod 104 includes a deflectable post 105 which may deflect relative to bone screw 120. Deflectable post 105 may deflect in a controlled manner relative to bone screw 120 thereby providing for load sharing at a spinal segment while preserving range of motion. The deflection rod includes a compliant member (not shown, but see, e.g., o-ring 206 of FIG. 2A) to modulate deflection of deflectable post 105 and may also include limit surfaces (not shown, but see, e.g., limit surface 213 of FIG. 2C) to constrain the deflection of deflectable post 105.

The bone anchor 100 provides stiffness and support where needed to support the loads exerted on the spine which the soft tissues of the spine are no longer able to support. Load sharing is enhanced by the ability to select the appropriate stiffness of the deflection rod in order to match the load sharing characteristics desired. The stiffness/flexibility of deflection of the deflectable post 105 relative to the bone screw 120 is adapted to be controlled and/or customized as will be described below. Deflection rods are, in some cases, formed separately from the bone screws and added to the bone screw before or after implantation. In some cases the deflection rod is integrated into the bone screw during manufacture, in which case portions of the deflection rod, such as the limit surface, are in some cases, provided by portions of the bone screw structure. For embodiments of this invention, the terms “deflection rod” and “loading rod” can be used interchangeably. In the embodiment of FIG. 1A, bone screw 120 is preferably assembled with deflection rod 104 during manufacture of bone anchor 100.

Bone screw 120 is an example of a component of the anchor system. Bone screw 120 includes a housing 130 at the proximal end. Housing 130 has a cavity 132 in the form of a bore which is coaxial with the longitudinal axis of bone screw 120 and open at the proximal end of the housing 130. As shown in FIG. 1A, bone screw 120 has a threaded shank 124 which engages a bone to secure the bone screw 120 onto a bone. Different anchoring components are, in some embodiments, used to anchor the system to different positions in the spine depending upon the anatomy and needs of the patient. For example, in embodiments of the invention the anchor system includes one or more alternative bone anchors known in the art e.g. bone hooks, expanding devices, barbed devices, threaded devices, sutures, staples, adhesive and other devices capable of securing a component to bone instead of or in addition to bone screw 120.

A collar 110 is adapted to secure the deflectable post 105 within cavity 132 of bone screw 120. Collar 110 is secured into a fixed position relative to bone screw 120 by threads and or a welded joint. As shown in FIG. 1A, bone screw 120 includes a housing 130 at the proximal end. Housing 130 includes a cavity 132 for receiving deflection rod 104. Cavity 132 is coaxial with threaded bone screw 120. The proximal end of cavity 132 is threaded (not shown) to receive and engage cap 210. In alternative embodiments different mechanisms and techniques are used to secure the deflection rod 104 to the bone screw 120 including for example, welding, soldering, bonding, and/or mechanical fittings including threads, snap-rings, locking washers, cotter pins, bayonet fittings or other mechanical joints.

As shown in FIG. 1A, deflection rod 104 and deflectable post 105 are oriented in a co-axial, collinear or parallel orientation to bone screw 120. This arrangement simplifies implantation, reduces trauma to structures surrounding an implantation site, and reduces system complexity. Arranging the deflectable post 105 co-axial with the bone screw 120 can substantially transfer a moment force applied by the deflectable post 105 from a moment force tending to pivot or rotate the bone anchor 100 about its axis, to a moment force tending to act perpendicular to the axis of the bone anchor 100. The deflection rod 104 thereby resists repositioning of the bone anchor 100 without the use of locking screws or horizontal bars to resist rotation. Moreover, because deflectable post 105 may undergo controlled deflection in response to loads exerted upon it by the vertical rod system, the deflectable post isolates the bone screw 120 from many loads and motions present in the vertical rod system.

Bone anchor 100 also includes a coupling 136 to which other components are adapted to be mounted. As shown in FIG. 1A, coupling 136 is the external cylindrical surface of housing 130. Bone anchor 100 thus provides two mounting positions, one being the mount 114 of deflectable post 105 and one being the surface of housing 130 (an external or offset mounting position). Thus, a single bone anchor 100 can serve as the mounting point for one, two or more components. A deflection rod 104 is adapted to be coaxially mounted in the cavity 132 of the housing 130 and one or more additional components are adapted to be externally mounted to the outer surface of the housing—coupling 136. For example, a component of the connection system is, in some embodiments, mounted to the outer surface/coupling 136 of the housing 130—such a connector is referred to herein as an offset head or offset connector (See, e.g. FIG. 1B).

FIG. 1B shows a component of the connection system which is, adapted to be mounted externally to the housing 130 of bone anchor 100. FIG. 1B shows a perspective view of offset connector 140 mounted externally to housing 130 of a bone anchor 100. Connector 140 is an example of an offset head or offset connector. Offset connector 140 comprises six components and allows for two degrees of freedom of orientation and two degrees of freedom of position in connecting a vertical rod or compound spinal rod to a bone anchor 100. The six components of offset connector 140 are dowel pin 142, pivot pin 144, locking set screw 146, plunger 148, clamp ring 141 and saddle 143. Saddle 143 has a slot 184 sized to receive a rod, for example, a vertical rod or compound spinal rod 150 of FIG. 1C. Locking set screw 146 is mounted at one end of slot 184 such that it is tightened to secure a rod within slot 184.

Clamp ring 141 is sized such that, when relaxed it can slide freely up and down the housing 130 of bone anchor 100 and rotate around the housing 130. However, when locking set screw 146 is tightened on a rod, the clamp ring 141 grips the housing and prevents the offset connector 140 from moving in any direction. Saddle 143 is pivotably connected to clamp ring 141 by pivot pin 144. Saddle 143 can pivot about pivot pin 144. However, when locking set screw 146 is tightened on a rod, the plunger 148 grips the clamp ring 141 and prevents further movement of the saddle 143. In this way, operation of the single set screw 146 serves to lock the clamp ring 141 to the housing 130 of the bone anchor 100, fix saddle 143 in a fixed position relative to clamp ring 141 and secure a rod within the slot 184 of offset connector 140.

The connector 140 of FIG. 1B is provided by way of example only. It is desirable to have a range of different connectors which are compatible with the anchor system and deflection system. The connectors have different attributes including, for example, different degrees of freedom, range of motion, and amount of offset which attributes more appropriate for a particular relative orientation and position of two bone anchor 100 and/or patient anatomy. Each connector is sufficiently versatile to connect a vertical rod to a bone anchor 100 in a range of positions and orientations while being simple for the surgeon to adjust and secure.

In preferred embodiments a set or kit of connectors is provided which allows the dynamic stabilization system to be assembled in a manner that adapts a particular dynamic stabilization prosthesis to the patient anatomy rather than adapting the patient anatomy for implantation of the prosthesis (for example by removing tissue\bone to accommodate the system). In a preferred embodiment, the set of connectors making up the connection system has sufficient flexibility to allow the dynamic stabilization system to realize a suitable dynamic stabilization prosthesis in all situations that will be encountered within the defined target patient population. Alternative embodiments of connection system components including coaxial heads and offset connectors can be found in the related patent applications incorporated by reference above.

A vertical rod or compound spinal rod is adapted to be connectable to mount 114 of deflectable post 105. FIG. 1C shows a perspective view of a compound spinal rod 150. Compound spinal rod 150 includes a first elongated rod 156a and a second elongate rod 156b. The rods 156a, 156b are preferably 5 mm titanium rods. First rod 156a is connected to second rod 156b by linkage 158. Linkage 158 allows controlled and constrained movement of rod 156a with respect to rod 156b. Rod 156a has a coupling 154a at one end for connecting compound spinal rod 150 to mount 114 of bone anchor 100. Rod 156b has a coupling 154b at one end for connecting compound spinal rod 150 to another bone anchor or connector (not shown). As shown in FIG. 1C, compound spinal rod 150 is mounted to a mount 114 of a bone anchor 100. Mount 114 is passed through an aperture in coupling 154a (not shown). A nut 160 is then secured to mount 114 securing coupling 154a to mount 114. In some embodiments coupling 154a permits compound spinal rod 150 to pivot and rotate relative to deflectable post 105. Note that a connector 140, such as shown in FIG. 1B, is adapted to be mounted to housing 130 to connect bone anchor 100 to a second vertical rod or compound spinal rod (not shown).

The components of the dynamic stabilization system are adapted to be assembled and implanted in the spine of a patient to provide a multilevel dynamic stabilization prosthesis which provides dynamic stabilization of the spine and load sharing. FIGS. 1D and 1E show posterior and lateral views of three adjacent vertebrae 191, 192 and 193. Referring first to FIG. 1D, as a preliminary step, bone anchors 100a, 100b, 100c, and 100d comprising deflection rods 104a, 104b, 104c and 104d and bone screws 120a, 120b, 120c, and 120d, have been implanted in vertebrae 191 and 192 on the left and right sides of the spinous process 194 between the spinous process 194 and the transverse process 195 in the pedicles 196 of each vertebra. In the example shown in FIG. 1D, polyaxial screws 106a, 106b are implanted in the pedicles 196 of vertebra 193.

In preferred procedures, the bone screw is directed so that the threaded portion is implanted within one of the pedicles 196 angled towards the vertebral body 197 of each vertebra. The threaded region of each bone screw is fully implanted in the vertebrae 191, 192. As shown in FIG. 1E, the bone screws 120a, 120b, 120c are long enough that the threaded portion of the bone screw extends into the vertebral body 197 of the vertebra. As shown in FIG. 1E, the housings 130a, 130b, 130c, 130d of each bone screw remain partly or completely exposed above the surface of the vertebrae so a connection system component can be secured to each bone screw 120a, 120b, 120c and 120d.

After installation of bone screws 120a, 120b, 120c, 120d and polyaxial screws 106a, 106b, the vertical rod system components and connection system components are installed and assembled. FIG. 1D shows, on the right side of the vertebrae, one way to assemble deflection system components and connection system components to form a dynamic stabilization prosthesis 160. (See also, lateral view of FIG. 1E). An offset connector 140d is shown mounted to housing 130d of bone screw 120d. A first compound spinal rod 150c is connected at one end to deflection rod 104c. Compound spinal rod 150c is connected at the other end by offset connector 140d to bone screw 120d. A second compound spinal rod 150d is connected at one end to deflection rod 104d. Compound spinal rod 150d is connected at the other end to polyaxial screw 106b.

The dynamic stabilization prosthesis 160 of FIG. 1D thus has a compound spinal rod 150c, 150d stabilizing each spinal level (191-192 and 192-193). Each of the compound spinal rods 150c, 150d is secured rigidly at one end to a bone screw (120b, 120c). Each of the compound spinal rods 150c, 150d is secured at the other end to a bone anchor 100c, 100d thereby allowing for some movement and load sharing by the dynamic stabilization prosthesis. Offset connector 140d permits assembly of the dynamic stabilization prosthesis for a wide range of different patient anatomies and/or placements of bone anchors 100a, 100b, 100c and 100d. An identical or similar dynamic stabilization prosthesis 160 would preferably be implanted on the left side of the spine. In alternative embodiments, a compound spinal rod is used at one level and a vertical rod which is not a compound spinal rod is used at an adjacent level.

In the embodiment shown in FIGS. 1A-1E, the bone anchors and compound spinal rods can be designed with different amounts of stiffness and range of motion by selecting among different deflection components. By selection of materials and dimensions, bone anchors and compound spinal rods can be provided in a range from a highly rigid configurations to very flexible configurations and still provide stabilization to the spine. Load sharing is enhanced by the ability to select the appropriate stiffness of the bone anchors and compound spinal rods in order to match the load sharing characteristics desired. By selecting the appropriate stiffness of the bone anchors and compound spinal rods to match the physiology of the patient and the loads that the patient places on the spine, a better outcome is realized for the patient.

The force/deflection curve of a bone anchor or compound spinal rod can be customized based on the choice of dimensions and materials. Furthermore, each of the bone anchors and compound spinal rods in the dynamic stabilization prosthesis can have a different stiffness, flexibility or range of motion. Thus, for, example, in one embodiment of a dynamic spinal stabilization prosthesis, a first bone anchor or compound spinal rod has a first stiffness, flexibility or range of motion, and a second bone anchor or compound spinal rod has a second different stiffness, flexibility or range of motion depending on the needs of the patient. In another embodiment, bone anchors and compound spinal rods have different stiffness, flexibility or range of motion properties for each level and/or side of the dynamic stabilization prosthesis depending on the user's needs. In other words, in some embodiments, one portion of a dynamic stabilization prosthesis offers more resistance to movement than another portion based on the design and selection of different bone anchors and compound spinal rods having different stiffness, flexibility or range of motion. Thus, in embodiments of the invention, the bone anchors and compound spinal rods can be made, selected and implanted so that the dynamic stabilization prosthesis replicates, for example, 70% of the range of motion and flexibility of the natural intact spine, 50% of the range of motion and flexibility of the natural intact spine and/or a 30% of the range of motion and flexibility of the natural intact spine.

The particular dynamic stabilization prosthesis 160 and components shown in FIGS. 1A-1E are provided by way of example only. It is an aspect of preferred embodiments of the present invention that a range of components be provided and that the components are adapted to be assembled in different combinations and organizations to create different assemblies suitable for the functional needs and anatomy of different patients. Dynamic stabilization is provided at one or more motion segments and in some cases dynamic stabilization is provided at one or more motion segments in conjunction with fusion at an adjacent motion segment. A particular dynamic stabilization prosthesis may incorporate various combinations of the bone screws, vertical rods, compound spinal rods, compound spinal rods, bone anchors, and connectors described herein and in the related applications incorporated by reference as well as standard spinal stabilization and/or fusion components, for example screws, rods and polyaxial screws.

FIGS. 2A-2E illustrate an embodiment of a bone anchor 200 having an integrated deflection rod 201 and bone screw 220 which is adapted to be utilized as part of a prosthesis for dynamic stabilization of the spine. A deflection rod 201 is incorporated into a bone screw 220 during manufacture. FIG. 2A shows an exploded view of bone anchor 200. As shown in FIG. 2A, deflection rod 201 includes four components: ball-shaped retainer 202, deflectable post 204, o-ring 206 and cap 210. FIG. 2B shows the bone anchor 200 after assembly. FIGS. 2C-2D show sectional views of bone anchor 200 and illustrate deflection of the deflectable post 204. FIG. 2E shows a sub-assembly of a dynamic spinal prosthesis incorporating bone anchor 200 and a compound spinal rod 150.

Referring first to FIG. 2A, bone anchor 200 includes a deflectable post 204 which has a retainer 202 at one end. Retainer 202 is a spherical structure formed in one piece with deflectable post 204. At the other end of deflectable post 204 is a mount 214. Mount 214, in this embodiment, is suitable for connecting to a vertical rod. In alternative embodiments, a ball is used in place of mount 214 as previously described. In this embodiment, mount 214 is also formed in one piece with deflectable post 204 and retainer 202. This piece is preferably made of cobalt chrome while, the rest of the bone anchor 200 is preferably made of titanium and/or stainless steel. The o-ring is made of a polymer as described below. In alternative embodiments, deflectable post 204 is formed separately from and securely attached to one or more of mount 214 and retainer 202 by laser welding, soldering or other bonding technology. Alternatively, deflectable post 204 is formed separately and mechanically engages one or more of mount 214 and retainer 202 using, for example, threads. For example, a lock ring, toothed locking washer, cotter pin or other mechanical device can be used to secure deflectable post 204 to one or more of mount 214 and retainer 202. As shown in FIG. 2A, mount 214 is a low profile mount configured to fit within a ball-joint 240 of a vertical rod component.

Bone anchor 200 includes a deflection rod 201 assembled with a bone screw 220, which comprises a bone screw 224 connected to a housing 230. Housing 230 has a cavity 232 oriented along the axis of bone screw 220 at the proximal end and configured to receive deflection rod 201. In other embodiments, housing 230 is longer while cap 210 is a smaller part. Cap 210, in this embodiment, is designed to perform multiple functions including holding o-ring 206 as well as securing retainer 202 in cavity 232 of bone screw 220. In the embodiment of FIG. 2A, cap 210 has an outer surface 234 adapted for mounting a component, e.g. an offset connector. Housing 230 may, in some embodiments, be cylindrical as previously described.

As also shown in FIG. 2A, outer surface 234 of housing 230 is provided with splines/flutes or registration elements 236. Splines/flutes 236 are adapted to be engaged by a driver that mates with splines/flutes 236 for implanting bone screw 220. Cap 210, by integrating the functions of the collar and sleeve, reduces the complexity of the deflection rod 201 and also increases the strength of the deflection rod 201 or allows a reduction in size for the same strength. Cap 210 comprises a cylindrical shield section 208 connected to a collar section 209. Cap 210 is designed to mate with cavity 232 of housing 230. The shield section 208 and collar section 209 are preferably formed in one piece. However, in alternative embodiments they are formed separately and then secured together. Shield section 208 is threaded adjacent collar section 209 in order to engage threaded cavity 232. Cap 210 may alternatively, or additionally, be joined to housing 230 by, for example, laser welding.

O-ring 206 has a round central aperture 207 for receiving the deflectable post 204 (see FIG. 2B). O-ring 206 fits within a groove 205 of cap 210 with the aperture 207 aligned with the central bore of cap 210 (see FIG. 2C). O-ring 206 is a compliant member which exerts a centering force upon deflectable post 204. Other shapes and configurations of compliant members are used in other embodiments, including, for example, tubes, sleeves and springs arranged to be compressed by deflection of the deflectable post 204 and exert a centering force upon deflectable post 204. O-ring 206 is preferably made from polycarbonate urethane (for example, Bionate®) or another hydrophilic polymer. This material is further described in U.S. Pat. No. 5,133,742, issued Jul. 28, 1992, and entitled and U.S. Pat. No. 5,229,431, issued Jul. 20, 1993, and entitled “Crack-Resistant Polycarbonate Urethane Polymer Prosthesis and the Like,” both of which are incorporated herein by reference.

Referring now to FIG. 2B, which shows a perspective view of bone anchor 200 having a deflection rod 201 assembled with a bone screw 220. When assembled, deflectable post 204 is positioned within cap 210 which is positioned within housing 230 of bone screw 220. O-ring 206 (See FIG. 2A) is first positioned within shield 208 of cap 210. Deflectable post 204 is then positioned through aperture 207 of o-ring 206 and cap 210. Deflectable post 204, o-ring 206 and cap 210 are then connected to cavity 232 of housing 230. The cap 210 is then secured to the threaded proximal end of cavity 232. Deflectable post 204 extends out of housing 230 and cap 210 such that mount 214 is accessible for connection to a compound spinal rod (not shown). There is a gap between deflectable post 204 and cap 210 which permits deflection of deflectable post 204 through a predefined range before deflection is limited by contact with cap 210.

Cap 210 has splines/flutes 236 for engagement by a wrench to allow cap 210 to be tightened to housing 230. Housing 230 is alternatively, or additionally, provided with splines/flutes or registration elements 236. The flutes/splines 236 are also useful to allow engagement of the cap/housing assembly by an implantation tool and/or by a connector. The flutes/splines or registration elements 236 allow the cap/housing to be gripped and have torque applied to allow implantation or resist rotation of a connector. Cap 210 may alternatively, or additionally, be laser welded to housing 230 after installation to secure the components. Cap 210 secures deflectable post 204 and o-ring 206 within cavity 232 of bone screw 220. (See FIG. 2C).

FIG. 2C shows a sectional view of a bone anchor 200. As shown in FIG. 2C, retainer 202 fits into a hemispherical pocket 239 in the bottom of cavity 232 of housing 230. The bottom edge of cap 210 includes a flange 215 which secures ball-shaped retainer 202 within hemispherical pocket 239 while allowing rotation of ball-shaped retainer 202. As shown in FIG. 2C, o-ring 206 occupies the space between deflectable post 204 and cap 210. In other embodiments, o-ring 206 may sit between deflectable post 204 and a housing of bone screw 220. O-ring 206 is secured within groove 205 of cap 210. O-ring 206 is compressed into groove 205. Groove 205 is, in some embodiments, slightly wider than necessary to accommodate o-ring 206 in order that o-ring 206 may expand axially while being compressed radially. The extra space in groove 205 reduces the possibility that o-ring 206 will become pinched between deflectable post 204 and the inside of cap 210. Cap 210 thereby secures both retainer 202 and o-ring 206 to housing 230.

O-ring 206 is compressed by deflection of deflectable post 204 towards shield 208 in any direction (see FIG. 2D). The o-ring 206 can act as a fluid lubricated bearing and allow the deflectable post 204 to also rotate about the longitudinal axis of the deflectable post 204 and the bone screw 220. Other materials and configurations can also allow the post to rotate about the longitudinal axis of the post and the bone screw.

FIG. 2D illustrates the deflection of deflectable post 204 of bone anchor 200 in response to a load placed on mount 214. Applying a force to mount 214 causes deflection of deflectable post 204. Initially, deflectable post 204 pivots about a pivot point 203 indicated by an X. Deflectable post 204 may pivot about pivot point 203 in any direction. Concurrently, or alternatively, deflectable post 204 can rotate about the long axis of deflectable post 204 (which also passes through pivot point 203). In this embodiment, pivot point 203 is located at the center of ball-shaped retainer 202. As shown in FIG. 2D, deflection of deflectable post 204 compresses the material of o-ring 206. The force required to deflect deflectable post 204 depends upon the dimensions of deflectable post 204, o-ring 206, groove 205 and shield 208 of cap 210 as well as the attributes of the material of o-ring 206. The o-ring 206 exerts a centering force back on deflectable post 204 pushing it back towards a position coaxial with bone screw 220.

After further loading and deflection, deflectable post 204 comes into contact with limit surface 213 of cap 210. Limit surface 213 is oriented such that when deflectable post 204 makes contact with limit surface 213, the contact is distributed over an area to reduce stress on deflectable post 204. After deflectable post 204 comes into contact with limit surface 213, further deflection requires deformation (bending) of deflectable post 204. Deflectable post 204 is relatively stiff, and the force required to deflect deflectable post 204 therefore increases significantly after contact of deflectable post 204 with cap 210. In a preferred embodiment, deflectable post 204 may deflect from 0.5 mm to 2 mm in any direction before making contact with limit surface 213. More preferably, deflectable post 204 may deflect approximately 1 mm before making contact with limit surface 213.

FIG. 2E illustrates the subassembly resulting from mounting connector 140 of FIGS. 1B, 1D and 1E to the housing of bone anchor 200 and also mounting compound spinal rod 150 of FIG. 1C. As shown in FIG. 2E, connector 140 connects bone anchor 200 to a compound spinal rod 250 (shown in part). Thus, bone anchor 200 is connected by compound spinal rods 150, 250 to other bone screws or bone anchors (not shown) on neighboring vertebrae to create a dynamic stabilization prosthesis which spans three vertebrae as illustrated, for example, in FIGS. 1D and 1E. Spinal 250 is in some cases identical to spinal rod 150. Spinal rod 250 is in alternative embodiments different than spinal rod 150. Spinal rod 150 and/or spinal rod 250 are in some embodiments replaced by conventional rigid spinal rods.

During implantation, connector 140 is adjusted to accommodate the angle from which compound spinal rod 250 approaches bone anchor 200. Note that connector 140 provides sufficient degrees of freedom to connect compound spinal rod 250 securely to housing 230. After adjustments are made, set screw 146 is tightened securing compound spinal rod 250 to saddle 143, locking the angle of saddle 143 relative to clamp ring 141, and securing clamp ring 141 to housing 230. Compound spinal rod 150 is connected to mount 214 of deflectable post 204 by coupling 154a such that compound spinal rod 150 can rotate about deflectable post 204 and pivot relative to deflectable post 204. Deflectable post 204 is also adapted to rotate within housing 230 of bone screw 220 and pivot relative to housing 230. The pivoting of deflectable post 204 is controlled and/or limited by components of bone anchor 200 as described in greater detail in the applications referred to above and incorporated by reference herein.

### Compound Spinal Rod

Vertical rods and/or compound spinal rods are used to span adjacent vertebra to provide stabilization. The vertical rods and compound spinal rods operate in conjunction with bone anchors to contribute to load sharing and motion preservation. In some embodiments, it is desirable to utilize compound spinal rods which have one or more degrees of freedom of movement in addition to or instead of the coupling connecting the compound spinal rod to the bone screw/bone anchor. Compound spinal rods include a first rod connected by a linkage to a second rod (see e.g. compound spinal rod 150 of FIG. 1C). The linkage allows for movement of the first rod relative to the second rod. The movement permitted by the compound spinal rod is designed to enhance the ability of a spinal stabilization prosthesis to more closely approximate the natural kinematics of the spine without impairing the stabilization of the spine. In some embodiments, compound spinal rods contribute to load sharing and motion preservation as part of a spinal stabilization prosthesis. In some embodiments, compound spinal rods also support increased interpedicular distance and forward translation of a vertebra during flexion of the spine.

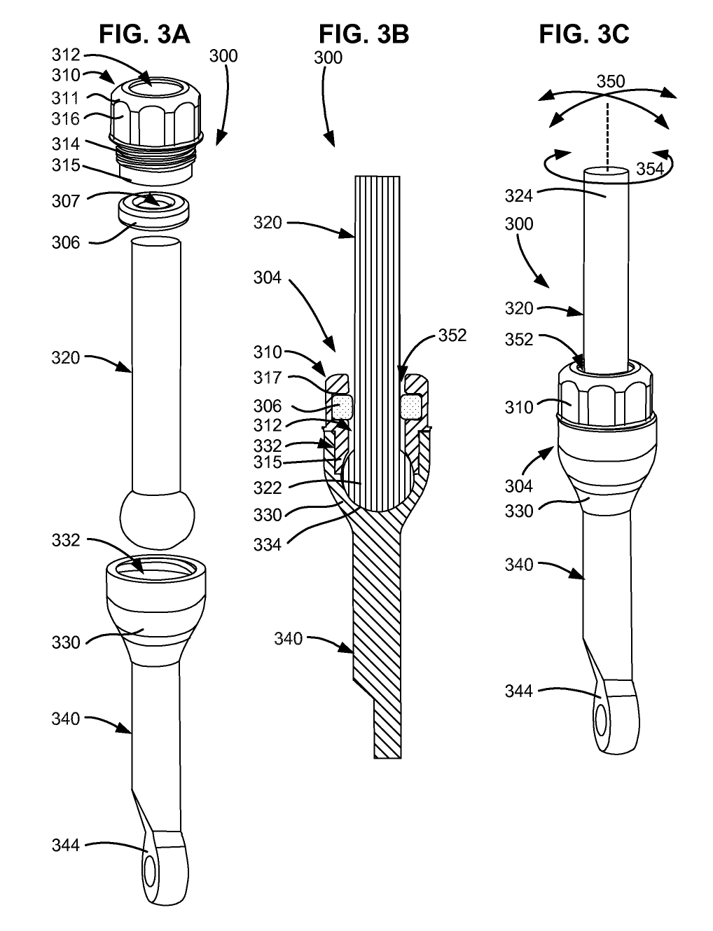

FIGS. 3A-3C illustrate the design and function of a compound spinal rod 300 according to an embodiment of the invention. FIGS. 3A-3C are exploded, sectional and perspective views of compound spinal rod 300. Referring first to FIG. 3A which shows the components of compound spinal rod 300. As shown in FIG. 3A, compound spinal rod 300 includes a first rod 320 and a second rod 340. Rod 320 includes a ball-shaped retainer 322 at one end (similar in design to retainer 202 of FIG. 2A) and a coupling 324 at the other end—in this case merely the cylindrical surface of the rod 320 to which a conventional pedicle screw can be mounted. Retainer 322 is preferably made of cobalt chrome. Rod 320 is preferably made in one piece including coupling 324 and retainer 322. Rod 340 has a housing 330 at one end and a coupling 344 at the other end. Housing 330 is similar in design to housing 230 of FIG. 2A. Rod 340 is preferably made in one piece including coupling 344 and housing 330. Compound spinal rod 300 also includes a cap 310 having a bore therethrough 312 (similar in design to cap 210 of FIG. 2A).

Compound spinal rod 300 includes an o-ring 306 (similar in design to o-ring 206 of FIG. 2A). O-ring 306 has a round central aperture 307 for receiving the rod 320 (see FIG. 2B).The o-ring is made of a hard-wearing compliant polymer. O-ring 306 is a compliant member which exerts a centering force upon rod 320 to keep it in alignment with rod 340.)-ring 306 is in some case round in section, square in section, or another shape compatible with the shape of groove 317 (see FIG. 3B). Other shapes and configurations of compliant members are used in other embodiments in place of o-ring 306, including, for example, tubes, sleeves and springs arranged to be compressed by deflection of the rod 320 and exert a centering force upon rod 320. O-ring 306 is preferably made from polycarbonate urethane (for example, Bionate®) or another hydrophilic polymer. This material is further described in U.S. Pat. No. 5,133,742, issued Jul. 28, 1992, and entitled and U.S. Pat. No. 5,229,431, issued Jul. 20, 1993, and entitled “Crack-Resistant Polycarbonate Urethane Polymer Prosthesis And The Like,” which is incorporated herein by reference. The o-ring 306 can act as a fluid lubricated bearing and allow the rod 320 to rotate about the longitudinal axis of the rod 320.

Housing 330 has a cavity 332 oriented along the axis of rod 340 and configured to receive retainer 322 and cap 310. Cap 310, in this embodiment, is designed to hold o-ring 306 in position around rod 320 as well as securing retainer 322 in cavity 332 of housing 330. O-ring 306 fits within a groove (not shown) of cap 310 with the aperture 307 aligned with the central bore 312 of cap 310 (see FIG. 3B). Cap 310 has an outer surface 316 which is shaped to allow cap 310 to be gripped by a tool for tightening cap 310 to housing 330. Cap 310 is designed to mate with cavity 332 of housing 330. Cap 310 includes a shield section 314 and collar section 311 that are preferably formed in one piece. Shield section 314 is threaded adjacent collar section 311 in order to engage cavity 332. Cap 310 is, in alternative embodiments, joined to housing 330 by, for example, laser welding.

Referring now to FIG. 3B, which shows a sectional view of compound spinal rod 300 as assembled. When assembled, O-ring 306 is first positioned within a groove 317 within cap 310. Rod 320 is then positioned in cap 310 through aperture 307 of o-ring 306 with coupling 324 passing out of central bore 312 of cap 310. Threaded sleeve 314 is then secured into cavity 332 of housing 330. The bottom edge of cap 310 includes a flange 315 which secures ball-shaped retainer 322 within hemispherical pocket 334 while allowing rotation of ball-shaped retainer 322. Cap 310 thus secures retainer 322 within housing 330 and holds o-ring 306 around rod 320. O-ring 306 is secured within groove 317 of cap 310. O-ring 306 is sized and configured such that o-ring 306 is compressed by deflection of rod 320 towards cap 310 in any direction.

Referring now to FIG. 3C which shows a perspective view of compound spinal rod 300 as assembled. Housing 330, retainer 322 and o-ring 306 (not shown) form a linkage 304 connecting rod 320 and rod 340 such that coupling 324 of rod 320 can move relative to coupling 344 of rod 340. Rod 340 is held in compliant alignment with rod 320 but can pivot a few degrees in any direction as shown by arrows 350 by compression of o-ring 306. Note that there is a gap 352 between rod 320 and cap 310 which permits deflection of rod 320 through a predefined range before deflection is limited by contact with cap 310. Rod 320 may also rotate 360 degrees about its long axis relative to rod 340 as shown by arrow 354. In this embodiment, the rod 320 pivots and rotates about axes which pass through the center of retainer 322. Compound spinal rod 300, by incorporating linkage 304, allows controlled and constrained motion between rod 320 and rod 340 thereby allowing for greater range of motion in a dynamic stabilization prosthesis and also reducing stresses on the dynamic stabilization prosthesis and the bones to which it is attached.

### Preserving Natural Motion of the Spine

With age, the vertebral bodies of the spine and intervertebral discs can degenerate resulting in discogenic instability. This spinal degeneration reduces the load-bearing ability of the spine, causes pain, reduces range of motion and can induce compensatory bone growth. The bone growth can lead to further reduction in range of motion and spinal stenosis in which the bone compresses blood vessels and nerves passing along the spine leading to inflammation and more pain.

A number of spinal prostheses have been proposed to maintain or restore the load-bearing capability of the spine, reduce discogenic instability, provide pain relief after discectomy, to top off degenerative discs above or below vertebral fusion, and/or to support degenerative discs without fusion. The basic objectives of such prostheses are load sharing and stabilization of the spine to remediate the problems identified above and reduce pain. However, the spine is a very complex structure and it is very difficult to provide a prosthesis for load sharing and stabilization that does not also change the natural kinematics of the spine causing additional artifacts, instabilities and as a result further degeneration of the spine. However, as described above, compound spinal rods and bone anchors are able to provide stabilization and load sharing with motion preservation.

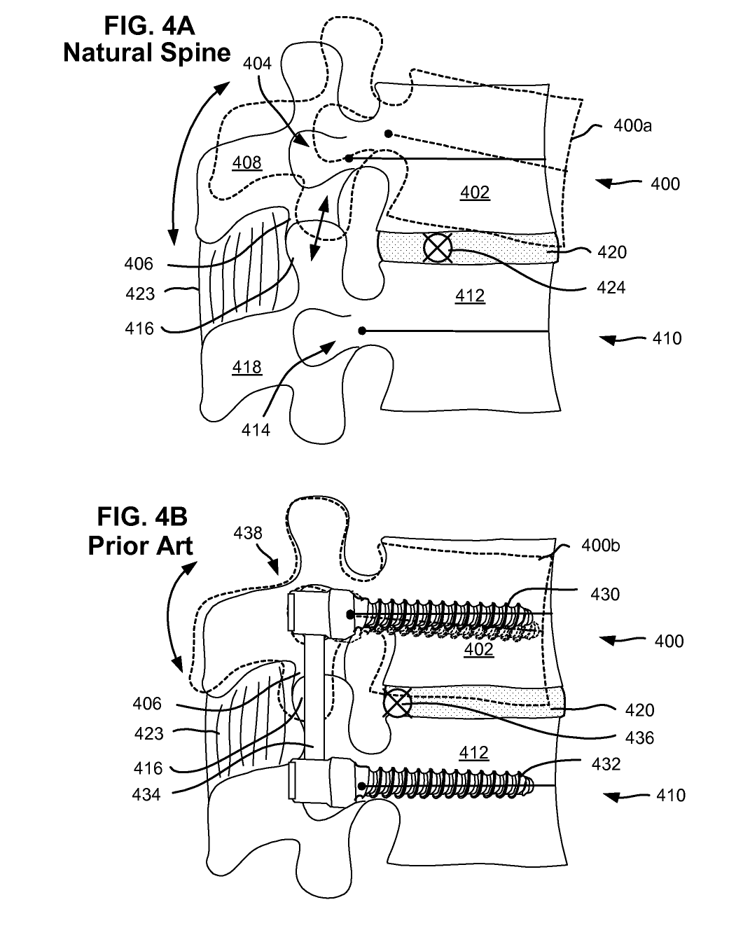

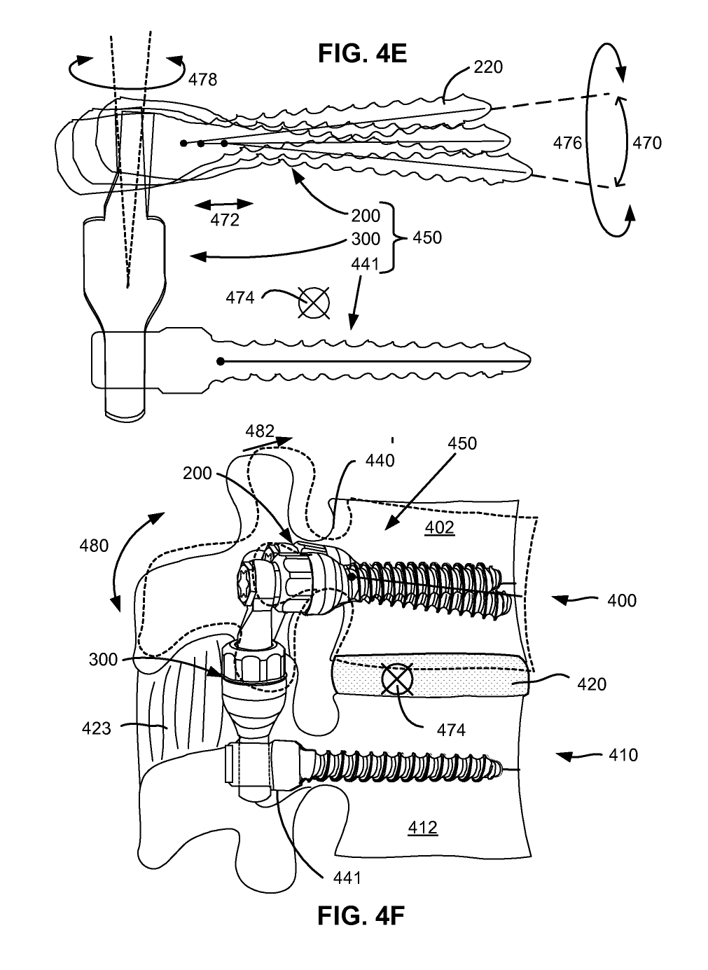

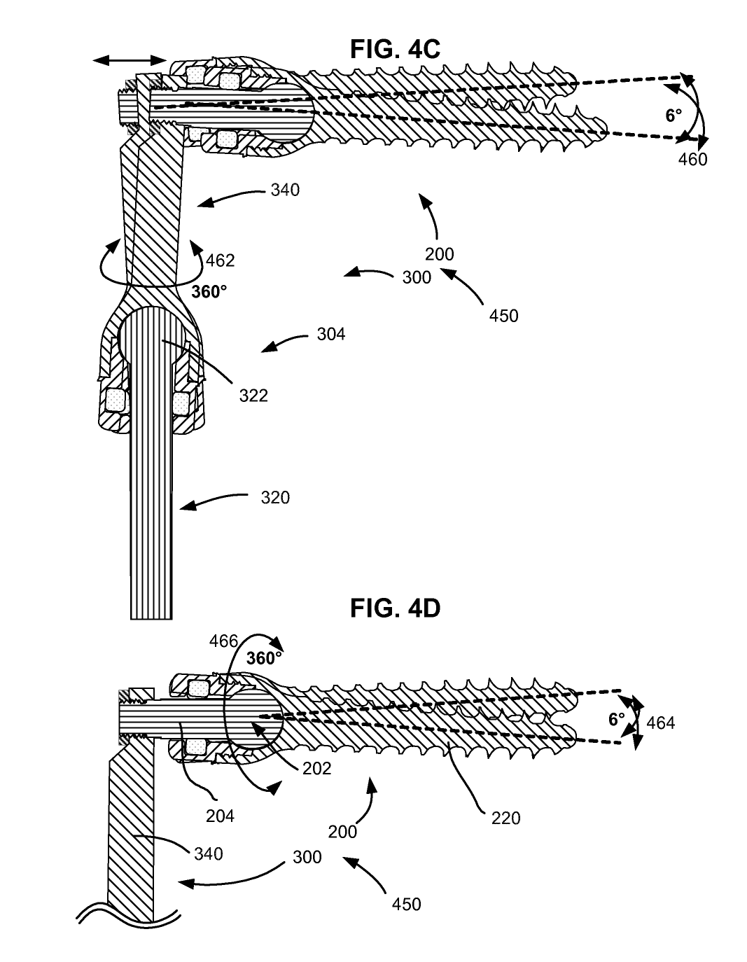

FIGS. 4A-4F illustrate and compare and contrast the motion constraints imposed by a rigid spinal stabilization prosthesis to the flexibility of a dynamic spinal stabilization prosthesis incorporating compound spinal rod 300 of FIGS. 3A-3C. Referring first to FIG. 4A which shows a lateral view of the lumbar spine illustrating the natural kinematics of the spine during extension and flexion. A superior vertebra 400 (for example L4) is shown relative to an inferior vertebra 410 (for example L5). The primary load bearing structures are the vertebral bodies 402 and 412. Between the vertebral bodies lies an intervertebral disc 420. Dorsal of the spinal bodies lie the pedicles 404, 414, facets 406, 416 and spinous processes 408, 418. Between the spinous process is a ligamentous band called the interspinous ligament 423.

As the spine flexes and extends the vertebrae move relative to one another while maintaining alignment of the vertebral bodies to support the weight of the upper body. In the healthy lumbar spine significant extension and flexion of the spine is possible in the lumbar region—approximating 45 degrees of total flexion over the entire lumbar region. Between extension and flexion, the superior vertebra 400 may move through an angle or range of about 15 degrees with respect to the inferior vertebra 410. In the healthy spine the natural center of rotation 424 for this rotation is located within the intervertebral disc 420. Rotation about the natural center of rotation 424 causes elongation of the interspinous ligament 423 and slight separation of the facets 406, 416. However, this rotation does not occur alone.

The healthy spine exhibits a phenomenon called coupling in which rotation or translation about or along one axis or plane is consistently associated with another motion about or along a second axis or plane. The dashed line 400a shows the position of the superior vertebra during flexion. As can be seen, during flexion, not only does the superior vertebra 400 rotate about the natural center of rotation 424, but it also translates cranially and dorsally. As a consequence, normal flexion also induces up to approximately an 8 mm increase in the distance between the pedicles 404, 414 from a combination of elevation and forward translation. This is enabled by elongation of the interspinous band and facet separation. Similarly, lateral bending of the spine is coupled with relative axial rotation of the vertebrae.

FIG. 4B is a lateral view of the lumbar spine illustrating the kinematic constraints placed on the spine by a rigid spinal prosthesis 438 during extension and flexion during extension and flexion. As shown in FIG. 4B, a pedicle screw 430 is implanted in the superior vertebra 400 and a pedicle screw 432 is implanted in the inferior vertebra 410. The pedicle screws are connected by a conventional rigid spinal rod or vertical rod 434. The vertical rod 434 and pedicle screws 430, 432 form a theoretically rigid spinal prosthesis 438 in that there are no joints/linkages which allow motion between any of the components after assembly. The vertical rod 434 transmits some of the load from the superior vertebra 400 to the inferior vertebra 410 thereby reducing the load on the vertebral bodies 402, 412 and the intervertebral disc 420.

During flexion of the spine, some rotation is permitted by flexing of the vertical rod 434 and the connections between the vertical rod 434 and the pedicle screws 430 and 432. The dashed lines 400b show the relative movement of the superior vertebra 400. However, the flexing of the vertical rod places significant strain upon the pedicle screws and the interface between the pedicle screws 430, 432 and the bone which can lead either to device failure, backing out of the screws or damage to the pedicles. Thus, an artifact of a rigid spinal prosthesis 438 as shown in FIG. 4B, is that the relative rotation of the vertebrae 499, 410 is constrained and the interpedicular distance is fixed.

As a result of the artifact introduced by the rigid spinal prosthesis 438, no elongation of the interspinous ligament 423 is possible and the center of rotation 436 is moved significantly dorsally of the natural center of rotation to the dorsal edge of the intervertebral disc or even further. Not only is facet separation prevented but the flexure about the new center of rotation can actually push the facets together increasing loading of the facet joints 406, 416. The rigid spinal prosthesis 438 also interferes with the natural coupling of the spine by precluding and/or limiting the translation of the superior vertebra which is normally associated with flexion. Furthermore, constraining motion at one segment of the spine is thought to create additional stress at adjacent segments and might therefore accelerate degeneration at those spinal segments (adjacent-level disease).

In order to overcome the problems caused by a rigid spinal prosthesis 438, a dynamic spine stabilization prosthesis attempts to preserve anatomical spinal motion and motion quality. An ideal prosthesis should be able to maintain intersegmental stability and permit appropriate motion at a spinal segment, e.g. ˜15 degrees of flexion/extension, ˜2 degrees of axial rotation, ˜6 degrees lateral bending as well as relative translation of the vertebrae ˜2 mm of left-right yaw, ˜2 mm of elevation (separation), and/or ˜2 mm of dorsal-ventral shift. The ideal prosthesis should also allow complex combinations of these motions and permit the coupling exhibited in the anatomical spine. The prosthesis should be able to preserve these motions required for normal spinal function while providing load sharing without abnormal load distribution, and spinal segment stabilization including limiting motion beyond anatomically desirable limits.

FIGS. 4C and 4D show the kinematic modes of a dynamic spine stabilization prosthesis 450 utilizing compound spinal rod 300 of FIGS. 3A-3C and bone anchor 200 of FIGS. 2A-2E in accordance with embodiments of the invention. FIGS. 4C and 4D show the kinematic modes of a bone anchor 200 in conjunction with a compound spinal rod 300. FIG. 4C shows the kinematic modes of bone anchor 200 relative to fixed rod 320 of compound spinal rod 300 assuming no motion internal to bone anchor 200. The movement is supported by linkage 304 of compound spinal rod 300. As shown in FIG. 4C, rod 340 pivots and rotates about ball 322 of rod 320. Rod 340 (and bone anchor 200) can pivot 3 degrees in any direction from perpendicular relative to fixed rod 320 of compound spinal rod as shown by arrow 460 for a total range of motion of 6 degrees. Rod 340 (and bone anchor 300) can also rotate 360 degrees relative to fixed rod 320 as shown by arrow 462.

FIG. 4D shows the kinematic modes of threaded anchor 220 relative to deflectable post 204 (and rod 340 of compound spinal rod 300) based solely on internal motion within bone anchor 200. As shown in FIG. 4D, threaded anchor 220 pivots and rotates about ball 202 of deflectable post 204. Threaded anchor 220 can pivot 3 degrees in any direction from perpendicular relative to deflectable post 204 as shown by arrow 464 for a total range of motion of 6 degrees. Threaded anchor 220 can also rotate 360 degrees relative to deflectable post 204 as shown by arrow 466.

The kinematics of the deflectable post 204 relative to rod 320 and of the threaded anchor 220 relative to deflectable post 204 combine to generate more complex kinematics than would be available with either component alone. The compound kinematics more closely approximate the natural kinematics of the spine. FIGS. 4E and 4F illustrate the compound kinematics of a dynamic stabilization prosthesis 450 incorporating a bone anchor 200 and compound spinal rod 300 and a conventional fixed bone screw 441.

Referring first to FIG. 4E which shows a simplified illustration of the kinematics of a dynamic spine stabilization prosthesis 450 showing the movement of bone anchor 200 and compound spinal rod 300 relative to fixed bone screw 441. As shown in FIG. 4E, the kinematics of the bone anchor 200 and compound spinal rod 300 combine to generate more complex kinematics than would be available with either component alone. Dynamic stabilization prosthesis 450 incorporating both the bone anchor 200 and compound spinal rod 300 allows not only a flexing motion (arrow 470) but also coupled translation (arrow 472) of a bone anchor 200 relative to a fixed bone screw 441. Moreover, the bone anchor may 200 may rotate around the axis of the compound spinal rod 300 as shown by arrow 478 permitting axial rotation of the spine. Additionally, the bone anchor may rotate around its own axis as shown by arrow 476 permitting lateral bending of the spine. The kinematics enabled by dynamic stabilization prosthesis 450 thus closely approximate the natural kinematics of the spine shown in FIG. 4A.

The pivoting motion and translation are coupled and compliantly modulated by the o-rings (not shown) of the bone anchor 200 and compound spinal rod 300. Moreover, the pivoting and translation are constrained by contact with the caps (not shown) of the bone anchor 200 and compound spinal rod 300 thus providing segmental stability. Furthermore the center of rotation 474 is maintained at an anatomically desirable position. Maintenance of a natural center of rotation 474 helps prevent uneven loading of the vertebral bodies 402, 412. The kinematics enabled by dynamic stabilization prosthesis 450 thus closely approximate the natural kinematics of the spine shown in FIG. 4A preserving the natural center of rotation while stabilizing the spine.

FIG. 4F is a lateral view of the spine illustrating the kinematics of a spinal segment supported by the dynamic spine stabilization prosthesis 450 of FIG. 4E. FIG. 4F shows a fixed bone screw 441 implanted in the inferior vertebra 410 and a bone anchor implanted in the superior vertebra 400. The fixed bone screw 441 is connected to the bone anchor 200 by compound spinal rod 300 to form a dynamic stabilization prosthesis 450. The compound spinal rod 300 transmits some of the load from the superior vertebra 400 to the inferior vertebra 410 thereby reducing the load on the vertebral bodies 402, 412 and the intervertebral disc 420. The compound spinal rod 300 also enables forward translation of the superior vertebra 400 relative to the inferior vertebra 410 coupled with flexion as shown by arrows 480 and 482. Furthermore the center of rotation 474 is maintained at an anatomically desirable position in the intervertebral disc 420. Maintenance of the natural center of rotation helps prevent uneven loading of the vertebral bodies 402, 412. The kinematics of the prosthesis by allowing translation of vertebra 400 relative to vertebra 410 also serves to preserve facet separation during flexion seen in the natural spine. Consequently, a dynamic spinal stabilization prosthesis incorporating both compound spinal rod 300 and bone anchor 200 can stabilize the spine and provide load sharing while preserving the natural kinetics of the spine (see FIG. 4A). Furthermore by allowing more natural kinematics, stain on the components and the bone interface is reduced leading to enhanced durability, safety and efficacy.

Referring again to FIG. 4F, the rotation of the bone anchor 200 around its axis and around the axis of the compound spinal rod 300 also permit kinematics impossible with rigid pedicle screw systems. For example, lateral bending of the spine may couple with relative rotation of the vertebrae 400, 410. In the rigid spinal implant of FIG. 4B, there is no provision for such rotation which would therefore resolve as strain upon the components and component/bone interface. However, dynamic stabilization prosthesis 450 allows both changes in the side-to-side intervertebral distance and coupled axial rotation of the vertebrae 400, 410 closely approximating the natural kinematics of the spine. Dynamic stabilization assemblies incorporating embodiments of the present invention can support complex combinations of natural movements and the coupled rotations and translations of the spine, for example, lateral bending with twisting, lateral bending with flexion. Thus, natural motion of the spine is stabilized and preserved.

The close approximation of the kinematics of the dynamic stabilization prosthesis 450 and the natural kinematics of the spine results in reduced stresses at the implant/bone interface and, by using a natural center of rotation, allows even stress distribution across the vertebral bodies and intervertebral disc. The prosthesis has a decreased stiffness and increased range of motion compared to conventional rigid vertical rod systems supporting the implant segment while reducing stresses on adjacent segments. The dynamic spine stabilization prosthesis, incorporating a compound spinal rod 300 and bone anchor is more robust than flexible rod systems. The degree of compliance in the compound spinal rod 300 and bone anchor 200 can also be tailored for the individual based upon load and anatomy. The result is anatomical load displacement curves, stabilization and preservation of natural motion and a robust surgical remediation of spinal degeneration.

### Alternative Compound Spinal Rods

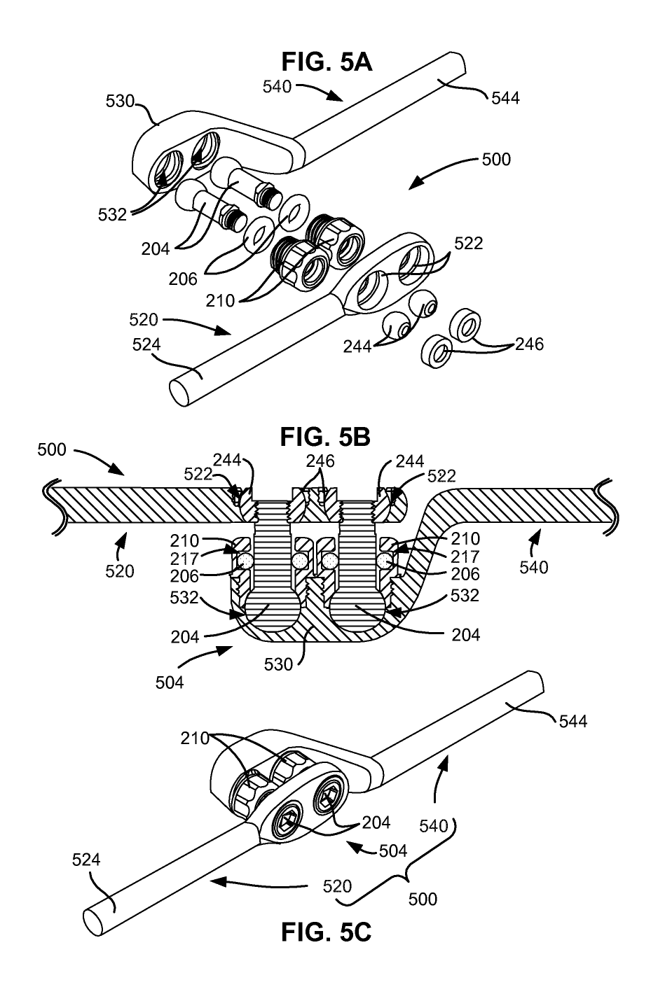

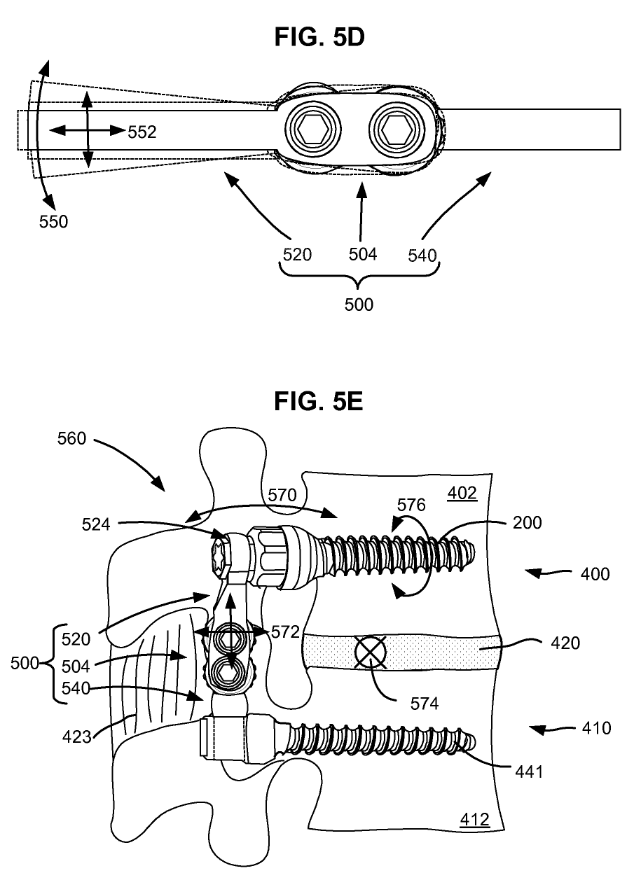

FIGS. 5A-5E illustrate the design and function of another compound spinal rod 500 according to an embodiment of the invention. FIGS. 5A-5C are exploded, sectional and perspective views of compound spinal rod 500. FIG. 5D shows the kinematic modes of the compound spinal rod of FIGS. 5A, 5B and 5C. FIG. 5E shows a lateral view of an example of a dynamic stabilization prosthesis incorporating compound spinal rod 500.

Referring first to FIG. 5A which shows the components of compound spinal rod 500. As shown in FIG. 5A, compound spinal rod 500 includes a first rod 520 and a second rod 540, two deflectable posts 204, two o-rings 206, two caps 210, two balls 244 and two races 246. Rod 540 includes a housing 530 at one end in which are two cavities 532, each configured to receive the deflectable posts 204, o-rings 206 and caps 210 in the manner described with respect to cavity 232 of FIGS. 2A-2D. Rod 540 is preferably made in one piece including coupling 544 and housing 530. Rod 520 includes two hemispherical pockets 522 at one end and a coupling 524 at the other end. The two hemispherical pockets 522 are configured to receive the balls 244 and races 246 in the manner described with respect to pocket 242 of FIGS. 2A-2D. Rod 520 is preferably made in one piece. Housing 530 has two cavities 532 oriented perpendicular to the axis of rod 540 and configured to receive deflectable posts 204, caps 210 and o-rings 206. Caps 210 are designed to hold o-rings 206 in position around deflectable posts 204 as well as securing deflectable posts 204 in cavities 532 of housing 530.

Referring now to FIG. 5B, which shows a sectional view of compound spinal rod 500 as assembled. When assembled, o-rings 206 are first positioned within grooves 217 within caps 210. Deflectable posts 204 are then positioned in caps 210 through o-rings 206. Caps 210 are the secured into cavities 532 of housing 530. Caps 210 thus secure deflectable posts 204 within housing 530 and hold o-rings 206 around deflectable post 204. Deflectable posts 204 can pivot and rotate relative to housing 530 as previously described. O-rings 206 are compressed by deflection of deflectable posts 204 and exert centering forces upon deflectable posts 204 to keep them perpendicular to rod 540. The balls 244 are received into pockets 522 of rod 520. The balls 244 are secured within pockets 522 by races 246 such that balls can pivot and rotate within pockets 522. The balls 244 are then secured to the ends of deflectable posts 204 which extend from caps 210. Housing 530, deflectable posts 204, o-rings 206, caps 210, balls 244, races 246 and pockets 522 form a linkage 504 connecting rod 520 and rod 540. The completed linkage 504 allows compliant and constrained movement of rod 520 relative to rod 540.

Referring now to FIG. 5C which shows a perspective view of compound spinal rod 500 as assembled. As shown in FIG. 5C, rod 540 is connected to rod 520 by linkage 504. Rod 540 is held in compliant alignment with rod 520 but can pivot a few degrees. Rod 540 can also translate relative to rod 520. The range of motion of rod 540 relative to rod 520 is constrained by caps 210 which limit the deflection of deflectable posts 204. By altering the dimensions of the caps 210 the range of motion is increased or decreased. The motion of rod 540 relative to rod 520 is also compliantly controlled by o-rings 206 (not shown) which apply centering forces upon deflectable posts 204 (See FIG. 5B). By changing the dimensions, design or material of o-rings 206 the amount of deflection of rod 540 can by changed for a given load. Thus linkage 504 can be manufactured to be stiffer or more compliant and the range of motion can be controlled as necessary or desirable for a particular application or patient. Compound spinal rod 500, by incorporating linkage 504, allows controlled motion between rod 520 and rod 540 thereby allowing for greater range of motion in a dynamic stabilization prosthesis and also reducing stresses on the dynamic stabilization prosthesis and the bones to which it is attached.

Referring now to FIG. 5D which shows the kinematics of compound spinal rod 500. As shown in FIG. 5D, rod 520 and rod 540 are connected by linkage 504. Rod 540 is held in compliant alignment with rod 520 but can pivot a few degrees in certain directions as shown by arrow 550. Rod 540 can also translate relative to rod 520 as shown by arrows 552. In some embodiments linkage 504 is configured so that translation is limited to extension of the compound spinal rod 500 and compression of compound spinal rod 500 is prevented. The range of motion of rod 540 relative to rod 520 is constrained by caps 210 and o-rings 206 which limit the deflection of deflectable posts 204 (See FIG. 5B). In this embodiment, the rod 520 pivots about an axis parallel to deflectable posts 204 and positioned midway between deflectable posts 204. Compound spinal rod 500, by incorporating linkage 504, allows controlled motion between rod 520 and rod 540 thereby allowing for greater range of motion in a dynamic stabilization prosthesis and also reducing stresses on the dynamic stabilization prosthesis and the bones to which it is attached.

FIG. 5E is a lateral view of two vertebrae 400, 410 of the spine showing an embodiment of a dynamic stabilization prosthesis 560 incorporating compound spinal rod 500. As shown in FIG. 5E, compound spinal rod 500 is connected at one end by coupling 524 to a bone anchor 200 and at the other end by coupling 544 to fixed bone screw 441. Coupling 524 is modified to connect to bone anchor 200 and may also include a ball-joint to permit pivoting and rotation of bone anchor 200 relative to rod 520. Dynamic stabilization prosthesis 560 supports some of the load transmitted from the superior vertebra 400 to the inferior vertebra 410 reducing stresses on the vertebral bodies 402, 412 and disc 420.

Dynamic stabilization prosthesis also compliantly supports and constrains relative movement of superior vertebra 400 relative to inferior vertebra 410. Dynamic stabilization prosthesis 560 incorporating both the bone anchor 200 and compound spinal rod 500 allows not only a flexing motion (arrow 570) but also coupled translation (arrows 572) of a bone anchor 200 relative to a fixed bone screw 441. Furthermore the center of rotation 574 is maintained at an anatomically desirable position. Maintenance of a natural center of rotation 574 helps prevent uneven loading of the vertebral bodies 402, 412. The pivoting motion and translation are coupled and compliantly modulated by the o-rings (not shown) of the bone anchor 200 and compound spinal rod 500. Moreover, the pivoting and translation are constrained by contact with the caps (not shown) of the bone anchor 200 and compound spinal rod 500 thus providing segmental stability. Additionally, the bone anchor 200 may rotate around its own axis as shown by arrow 576 permitting lateral bending of the spine. The kinematics enabled by dynamic stabilization prosthesis 560 thus closely approximate the natural kinematics of the spine shown in FIG. 4A. The deflection/force response for each of the movement modes of the dynamic stabilization prosthesis can be controlled by controlling the force/deflection properties and range of motion of the compound spinal rod 500 and bone anchor 200 as previously discussed.

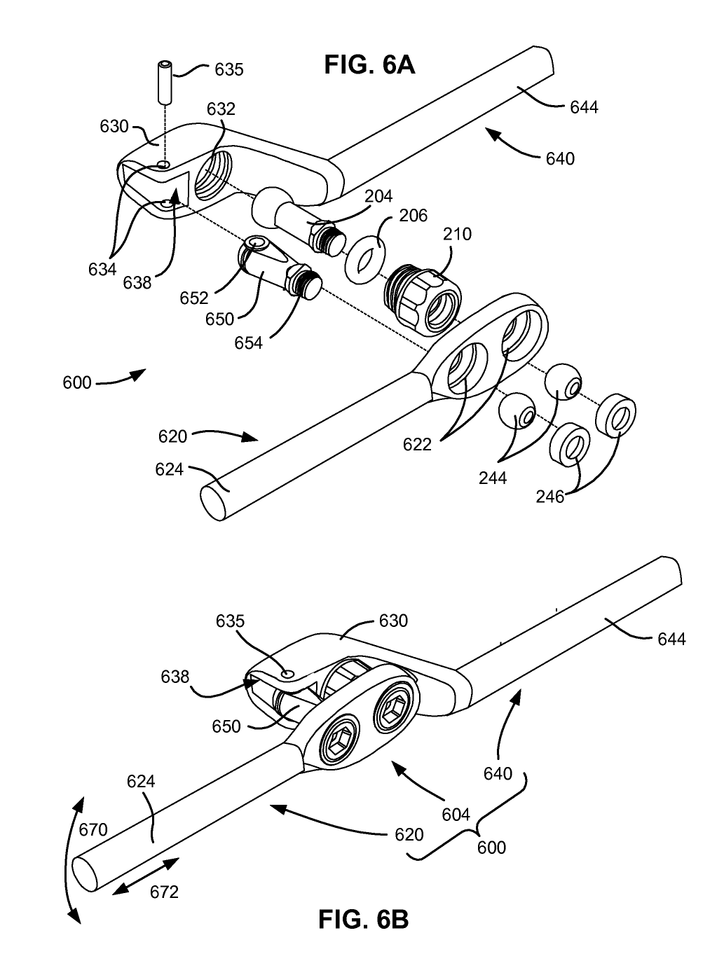

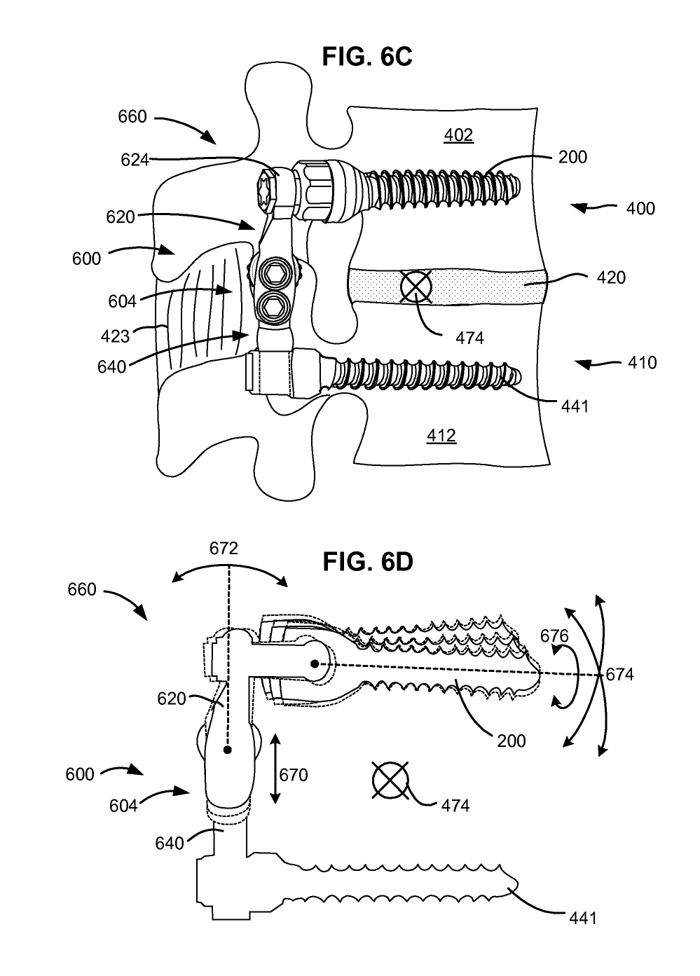

FIGS. 6A-6D illustrate the design and function of another compound spinal rod 600 according to an embodiment of the invention. FIGS. 6A and 6B are exploded and perspective views of compound spinal rod 600. FIG. 6C shows a lateral view of an example of a dynamic stabilization prosthesis 660 incorporating compound spinal rod 600. FIG. 6D shows the kinematic modes of the dynamic stabilization prosthesis 660 of FIG. 6C.

Referring first to FIG. 6A which shows the components of compound spinal rod 600. As shown in FIG. 6A, compound spinal rod 600 includes a first rod 620 and a second rod 640, deflectable post 204, o-ring 206, cap 210, pivot rod 650, pin 635, two balls 244 and two races 246.

Rod 640 includes a housing 630 at one end in which there is one cavity 632 and one slot 638. Cavity 632 is configured to receive the deflectable post 204, o-ring 206 and cap 210 in the manner described with respect to cavity 532 of FIGS. 5A-5C. Rod 640 is preferably made in one piece including coupling 644 and housing 630. Housing 630 has one cavity 632 oriented perpendicular to the axis of rod 640 and configured to receive deflectable post 204, cap 210 and o-ring 206. Cap 210 is designed to hold o-ring 206 in position around deflectable post 204 as well as securing deflectable post 204 in cavities 632 of housing 630.

During assembly, o-ring 206 is first positioned within cap 210. Deflectable post 204 is then positioned in cap 210 through o-ring 206. Cap 210 is then secured into cavity 632 of housing 630. Cap 210 thus secures deflectable post 204 within housing 630 and holds o-ring 206 around deflectable post 204. Deflectable post 204 can pivot and rotate relative to housing 630 as previously described. In this embodiment, pivot rod 650 replaces the second deflectable post of the embodiment of FIGS. 5A-5E. Pivot rod 650 is received in slot 638 of housing 630. Pivot rod 650 has an aperture 652 for receiving pin 635. Pin 635 passes through apertures 634 of housing 630 securing pivot rod 650 into slot 638. Pivot rod 650 may pivot around the axis of pin 635 but that is the sole degree of freedom of motion.

Rod 620 includes two hemispherical pockets 622 at one end and a coupling 624 at the other end. The two hemispherical pockets 622 are configured to receive the balls 244 and races 246 in the manner described with respect to pockets 522 of FIGS. 5A-5C. Rod 620 is preferably made in one piece. The balls 244 are received into pockets 622 of rod 620. The balls 244 are secured within pockets 622 by races 246 such that balls can pivot and rotate within pockets 622. The balls 244 are then secured to the ends of deflectable post 204 and pivot rod 650. Housing 630, deflectable posts 204, o-rings 206, caps 210, balls 244, races 246 and pockets 622 form a linkage 604 connecting rod 620 and rod 640. The completed linkage 604 allows constrained movement of rod 620 relative to rod 640.

Referring now to FIG. 6B which shows a perspective view of compound spinal rod 600 as assembled. As shown in FIG. 6C, rod 640 is connected to rod 620 by linkage 604. Rod 640 is held in compliant alignment with rod 620 but can pivot a few degrees in certain directions as shown by arrow 650. Rod 640 can also translate relative to rod 620 as shown by arrow 672. However the translation is limited to extension or compression of compound spinal rod 600 because there is no lateral deflection of pivot rod 650. In some embodiments linkage 604 is configured so that translation is limited to extension of the compound spinal rod 600 and compression of compound spinal rod 600 is prevented. The range of motion of rod 640 relative to rod 620 is constrained by caps 210 and o-rings 206 which limit the deflection of deflectable posts 204 (See FIG. 6B). In this embodiment, the rod 620 pivots about the axis of pivot rod. Compound spinal rod 600, by incorporating linkage 604, allows controlled motion between rod 620 and rod 640 thereby allowing for greater range of motion in a dynamic stabilization prosthesis and also reducing stresses on the dynamic stabilization prosthesis and the bones to which it is attached.

FIG. 6C is a lateral view of two vertebrae 400, 410 of the spine showing an embodiment of a dynamic stabilization prosthesis 660 incorporating compound spinal rod 600. As shown in FIG. 6C, compound spinal rod 600 is by coupling 624 to bone anchor 200 and at the other end to fixed bone screw 441. Note that coupling 624 is adapted in the case to be secured to the mount (not shown) of bone anchor 200. Coupling 624 may simply be a bore sized to receive the mount (not shown) or may comprise a ball-joint for allowing pivoting and/or rotation at the connection between rod 620 and bone anchor 200.

FIG. 6D shows the principal modes in which dynamic stabilization prosthesis 660 incorporating compound spinal rod 600 can move. As shown in FIG. 6D, the dynamic stabilization prosthesis 660 supports extension and compression of compound spinal rod 600 as shown by arrow 670 corresponding to stretching and compression of the interspinous ligament 423. Dynamic stabilization prosthesis 660 also supports pivoting of rod 620 relative to rod 640 as shown by arrow 672. Relative movement of the rod 640 and rod 620 in each of these modes requires deflection of the deflectable post 204 and compression of o-ring 206 (not shown) of compound spinal rod 600. The deflection/force response for each of the movement modes of the compound spinal rod 600 can, therefore, be controlled by controlling the force/deflection properties of the deflectable post 204 in the manner previously discussed. The compound spinal rod 600 will be more constrained with respect to the bending modes compared to compound spinal rod 500 because the pivot rod is constrained to a single axis of movement. Also as previously discusses bone anchor may pivot and rotate relative to rod 620 as shown by arrows 674 and 676.

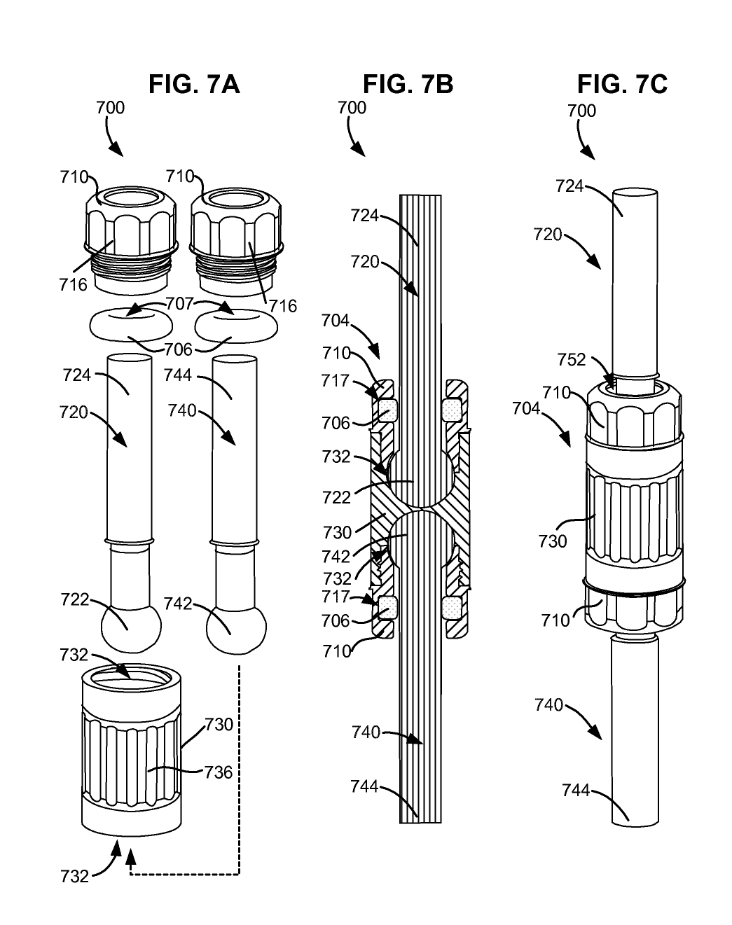

FIGS. 7A-7C illustrate the design and function of another compound spinal rod 700 according to an embodiment of the invention. FIGS. 7A-7C are exploded, sectional and perspective views of an alternative compound spinal rod 700 and its components. Referring first to FIG. 7A which shows the components of compound spinal rod 700. As shown in FIG. 7A, compound spinal rod 700 includes a first rod 720, a housing 730, and a second rod 740. Rods 720 and 740 include ball-shaped retainers 722, 742 at one end (similar in design to retainer 202 of FIG. 2A) and couplings 724, 744 at the other end—in this case merely the cylindrical surface of the rods 724, 744 to which a conventional pedicle screw can be mounted. Retainers 722, 742 are preferably made of cobalt chrome. Rods 720, 740 are preferably made in one piece including couplings 724, 744 and retainers 722, 742. Housing 730 is generally cylindrical with a cavity 732 in each end similar to the cavity 232 of FIG. 2A. Compound spinal rod 700 also includes two caps 710 having a bore therethrough (similar in design to cap 210 of FIG. 2A) and two o-rings 706 (similar in design to o-ring 206 of FIG. 2A). O-rings 706 have round central apertures 707 for receiving the rods 720 and 740 (see FIG. 2B).The o-rings 706 are made of a hard-wearing compliant polymer.

Housing 730 has a cavity 732 at each end oriented along the axis of rod 740 and configured to receive retainers 722, 742 and caps 710. Caps 710 are designed to hold o-rings 706 in position around rods 720, 740 as well as securing retainers 722, 742 in cavities 732 of housing 730. Caps 710 each have an outer surface 716 which is shaped to allow the surface 716 to be gripped by a tool for tightening cap 710s to housing 730. Housing 730 similarly has an outer surface 736 which is shaped to allow housing 730 to be gripped by a tool. Caps 710 are designed to mate with cavities 732 as previously described.

Referring now to FIG. 7B, which shows a sectional view of compound spinal rod 700 as assembled. During assembly, o-rings 706 are first positioned within grooves 717 within caps 710. Rods 720, 740 are then each positioned in a cap 710 through apertures 707 of o-rings 706 with couplings 724, 744 passing out of the central bores of the caps 710. The caps 710 are then secured to the cavities 732 of housing 730. The caps 710 secure retainers 722, 724 within housing 730 and hold o-rings 706 around rods 720, 740 while allowing rotation of ball-shaped retainers 722, 724 and pivoting of rods 720, 740 relative to housing 730.

As shown in FIG. 7B, o-rings 706 are secured within grooves 717 of caps 710. O-rings 706 are sized and configured such that o-rings 706 are compressed by deflection of rods 720, 740 towards caps 710 in any direction. O-rings 706 exert a centering forces upon rods 720, 740 to align them with housing 730 and each other. Other shapes and configurations of compliant members are used in other embodiments, including, for example, tubes, sleeves and springs arranged to be compressed by deflection of the rods 720, 740 and exert a centering force upon them. The o-rings 706 can act as a fluid lubricated bearing and allow the rods 720, 740 to also rotate about the longitudinal axis of the rods 720, 740 relative to housing 730 and each other. Housing 730, caps 710, retainers 722, 724 and o-rings 706 form a linkage 704 connecting rod 720 and rod 740 such that the coupling 724 of rod 720 may move relative to the coupling 744 of rod 740.

Referring now to FIG. 7C which shows a perspective view of compound spinal rod 700 as assembled. Housing 730, o-rings 706, caps 710 and retainers 722, 742 form a linkage 704. Linkage 704 allows compliant and constrained movement of coupling 72 relative to coupling 744. Rod 740 is held in compliant alignment with rod 720 but both rods 720, 740 may pivot a few degrees in any direction with respect to housing 730 and each other by compression of o-rings 706. Note that deflection of rods 720, 740 is limited by contact with caps 710. Note that there is a gap 752 between rod 720 and cap 710 and a similar gap 752 between rod 740 and cap 710 which permits deflection of rods 720 and 740 through a predefined range before deflection is limited by contact with caps 710. Rods 720 and 740 may also rotate 360 degrees about their long axis relative to housing 730 and each other. In this embodiment, the rods 720, 740 pivot and rotate relative to housing 730 about axes which pass through the centers of retainer 722, 724. Compound spinal rod 700 is adapted to be incorporated into a dynamic stabilization prosthesis in a similar manner to the compound spinal rods previously described. Compound spinal rod 700, by incorporating linkage 704, allows controlled motion between rod 720 and rod 740 thereby allowing for greater range of motion in a dynamic stabilization prosthesis and also reducing stresses on the dynamic stabilization prosthesis and the bones to which it is attached. Compound spinal rod 700 is adapted to be incorporated into a dynamic stabilization prosthesis in a similar manner to the compound spinal rods previously described. Compound spinal rod 700, by incorporating linkage 704, allows controlled motion between rod 720 and rod 740 thereby allowing for greater range of motion in a dynamic stabilization prosthesis and also reducing stresses on the dynamic stabilization prosthesis and the bones to which it is attached.

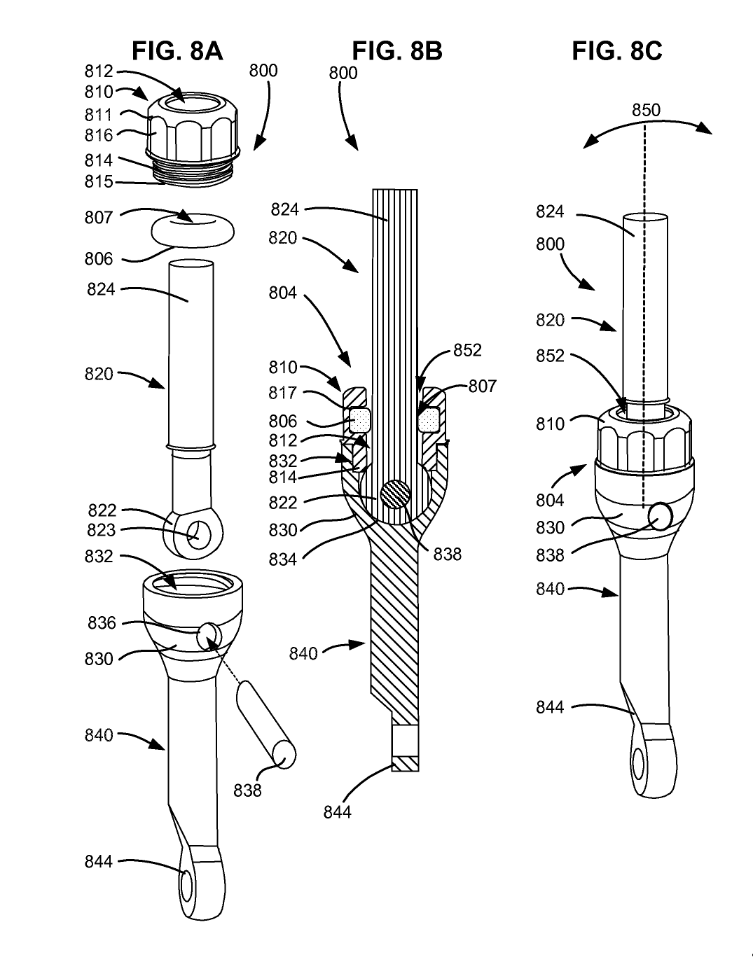

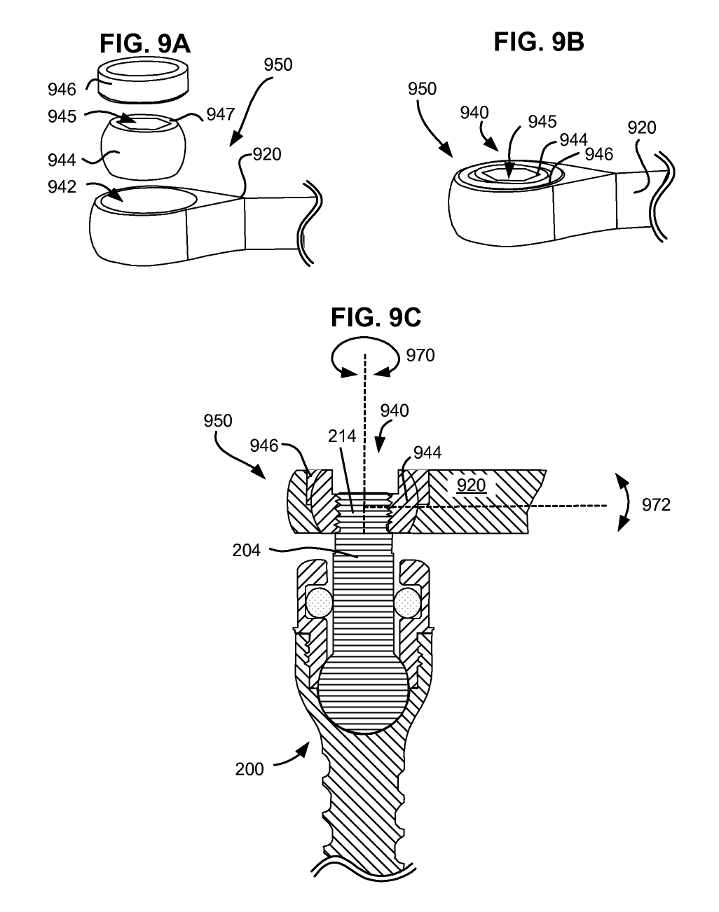

Compound spinal rod 700 can be utilized in the prostheses, linkages, and assemblies as described above and illustrated for example in FIGS. 1D, 1E, 2E, 4C, 4D, 5E, 6C and 6D and accompanying text. Compound spinal rod can be modified through the use of different couplings on the rods including rods, apertures, ball-joints pivoting joints and the like as shown for example in FIGS. 8A and 9A-9C.

FIGS. 8A-8C illustrate the design and function of another compound spinal rod 800 according to an embodiment of the invention. FIGS. 8A-8C are exploded, sectional and perspective views of compound spinal rod 800.

Referring first to FIG. 8A which shows the components of compound spinal rod 800. As shown in FIG. 8A, compound spinal rod 800 includes a first rod 820 and a second rod 840. Rod 820 includes a disc-shaped retainer 822 at one end and a coupling 824 at the other end. Retainer 822 is preferably made of cobalt chrome. Rod 820 is preferably made in one piece including coupling 824 and retainer 822. Rod 840 has a housing 830 at one end and a coupling 844 at the other end. Housing 830 is similar in design to housing 230 of FIG. 2A. However housing 830 is adapted to mate with disc-shaped retainer 822. Housing 830 also includes a transverse bore 836 for receiving a pin 838. Rod 840 is preferably made in one piece including coupling 844 and housing 830. Compound spinal rod 800 also includes a cap 810 having a bore therethrough 812 (similar in design to cap 210 of FIG. 2A) and an compliant member 806 (similar in design to o-ring 206 of FIG. 2A). Compliant member 806 has a round central aperture 807 for receiving the rod 820 (see FIG. 2B).The compliant member 806 is made of a hard-wearing compliant polymer. The compliant member need not be a ring as deflection of rod 820 will be constrained by pin 838 to a single axis.

Housing 830 has a cavity 832 oriented along the axis of rod 840 and configured to receive retainer 822 and cap 810. Cap 810, in this embodiment, is designed to hold compliant member 806 in position around rod 820. Disc-shaped retainer 822 is held in cavity 832 by a pin which passes through transverse bore 836 and disc bore 823. Cap 810 has an outer surface 816 which is shaped to allow cap 810 to be gripped by a tool for tightening cap 810 to housing 830. Cap 810 is designed to mate with cavity 832 of housing 830. Cap 810 includes a shield section 814 and collar section 811 that are preferably formed in one piece. Shield section 814 is threaded adjacent collar section 811 in order to engage cavity 832. Cap 810 may alternatively, or additionally, be joined to housing 830 by, for example, laser welding. Compliant member 806 fits within a groove 817 of cap 810 with the aperture 807 aligned with the central bore 812 of cap 810 (See FIG. 8B).

Referring now to FIG. 8B, which shows a sectional view of compound spinal rod 800 as assembled. When assembled, compliant member 806 is positioned within groove 817 within cap 810. Rod 820 is then positioned in cap 810 through aperture 807 of compliant member 806 with coupling 824 passing out of central bore 812 of cap 810. Threaded sleeve 814 is then secured into cavity 832 of housing 830. Cap 810 thus holds compliant member 806 around rod 820. Pin 838 passes through disc bore 823 to secure disc-shaped retainer 822 within a complementary pocket 834 of cavity 832 while allowing rotation of disc-shaped retainer 822 about the axis of pin 838. As shown in FIG. 8B, compliant member 806 is secured within groove 817 of cap 810. Compliant member 806 is sized and configured such that compliant member 806 is compressed by deflection of rod 820 towards cap 810. Compliant member 806 exerts a centering force upon rod 820 to keep it in alignment with rod 840.

Referring now to FIG. 8C which shows a perspective view of compound spinal rod 800 as assembled. Housing 830, disc-shaped retainer 822, cap 810, pin 838 and compliant member 806 form a linkage 804 connecting rod 820 and rod 840 such that coupling 824 of rod 820 may move relative to coupling 844 of rod 840. Rod 840 is held in compliant alignment with rod 820 but can pivot a few degrees around pin in any direction as shown by arrows 850 by compression of compliant member 806. Note that there is a gap 852 between rod 820 and cap 810 which permits deflection of rod 820 through a predefined range before deflection is limited by contact with cap 810. Compound spinal rod 800 is adapted to be incorporated into a dynamic stabilization prosthesis in a similar manner to the compound spinal rods previously described. Compound spinal rod 800, by incorporating linkage 804, allows controlled motion between rod 820 and rod 840 thereby allowing for greater range of motion in a dynamic stabilization prosthesis and also reducing stresses on the dynamic stabilization prosthesis and the bones to which it is attached. Compound spinal rod 800 can be utilized in the prostheses, linkages, and assemblies as described above and illustrated for example in FIGS. 1D, 1E, 2E, 4C, 4D, 5E, 6C and 6D and accompanying text. Compound spinal rod can be modified through the use of different couplings on the rods including rods, apertures, ball-joints pivoting joints and the like as shown for example in FIGS. 9A-9C.

### Couplings for Compound Spinal Rods

FIGS. 9A-9C illustrate alternative couplings adapted to connect a rod of a compound spinal rod to a post/deflectable post of a bone screw or bone anchor. FIG. 9A shows an exploded view of rod coupling 950. FIG. 9B shows a perspective view of the rod coupling 950. FIG. 9C show sectional views of rod coupling 950 illustrating the kinematics of the coupling with respect to a deflectable post.

Referring first to FIG. 9A which shows the components of a preferred embodiment of a rod coupling 950 for use with a compound spinal rod. Rod coupling 950 includes a ball 944 and race 946. Ball 944 is preferably made of cobalt chrome alloy for better wear. Ball 944 may alternatively be made of titanium or titanium alloy with a cobalt chrome coating. Ball 944 has a central aperture 945 designed to be secured to a threaded post. Central aperture 945 is threaded to enable ball 944 to be secured to the threads of a threaded post (not shown). Central aperture 945 also has a female hex socket 947 which may mate with a wrench in order to tighten ball 944 to the threaded end of a post. Ball 944 is received in a spherical pocket 942 in the end of a rod 920. Ball 944 is secured in spherical pocket 942 by race 946. Race 946 is secured to vertical rod 950 by, for example, threads and/or laser welding. When secured, ball 944 may rotate and pivot in the spherical pocket 942. Advantageously, there is no nut extending beyond ball 944 thus reducing the profile of the connection between mount 914 and vertical rod 950. To put it another way, the ball 944 acts as its own nut to secure ball 944 to a threaded post. Ball joint 940 allows greater range of motion and reduces torsional stresses on the dynamic stabilization assembly and the bones to which it is attached.

FIG. 9B shows a perspective view of rod coupling 950. Rod coupling 950 is assembled by placing ball 944 in pocket 942 of rod 920. Race 946 is then secured into pocket 942 by threads and/or laser welding. Race 946, ball, 944 and pocket 942 form a ball-joint 940 once assembled. Ball 944 is trapped in the spherical pocket formed by pocket 942 and race 946 but is free to pivot and rotate within the pocket. Central aperture 945 is accessible from either end of pocket 942 for attachment to the post of a bone screw or bone anchor.

FIG. 9C shows a sectional view of coupling 950 assembled with bone anchor 200 of FIGS. 2A-2E. FIG. 9C. As shown in FIG. 9C, ball 944 is secured to the mount 214 of deflectable post 204. To attach the coupling 950 to a post of a bone screw or bone anchor, ball 944 is threaded onto the threads of a threaded mount and tightened into place. When coupling 950 is secured to deflectable post 204, rod 920 may rotate 360 degrees around ball 944 as shown by arrow 970. Rod 920 may also pivot around ball 944 up to 15 degrees from perpendicular to deflectable post 204. Coupling 950 thereby allows for greater range of motion in a dynamic stabilization prosthesis and also reduces stresses on a dynamic stabilization prosthesis and the bones to which it is attached.

Coupling 950 is adapted to be incorporated as the coupling of one or more rods of the compound spinal rods previously described. The pocket 942 is preferably formed in one piece with the rod for assembly of the coupling 950, however in some cases the coupling is formed and assembled separately from the rod and then attached to the rod. In alternative embodiments, coupling 950 is adapted to be secured by a separate nut or other separate fastener to a post or deflectable post. Also, in alternative embodiments coupling 950 is configured to allow pivoting but not rotation or to allow rotation but not pivoting.

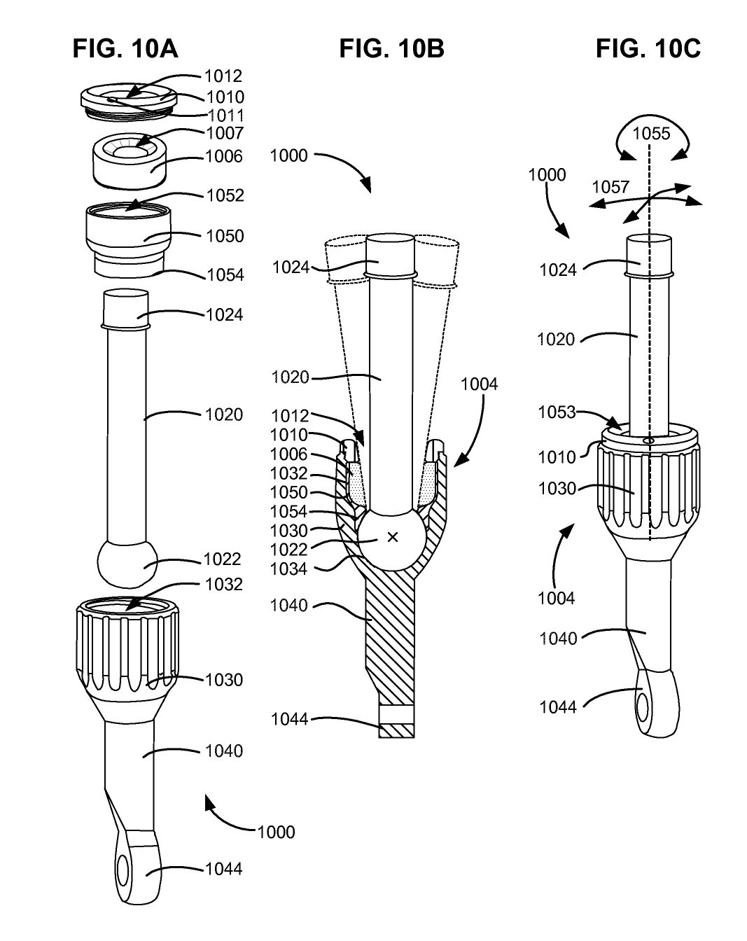

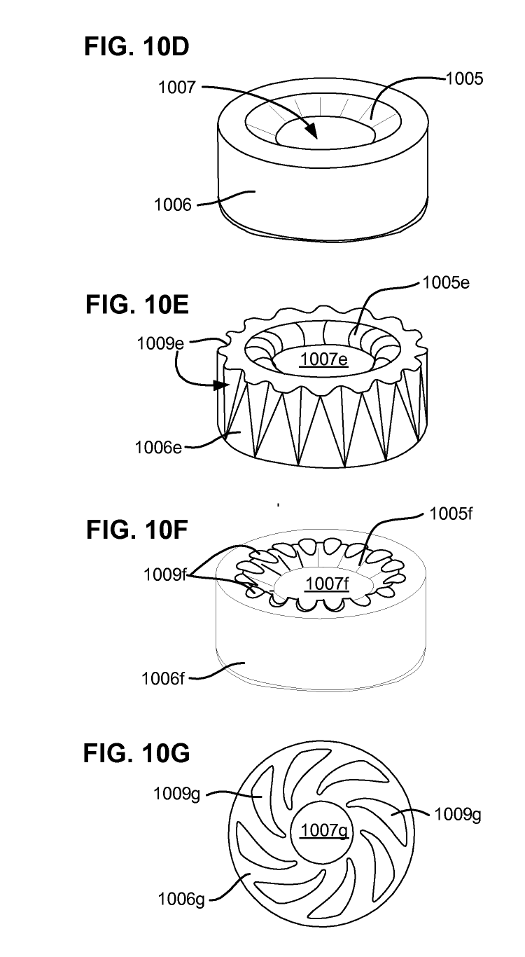

FIGS. 10A-10C are exploded, sectional and perspective views of an alternative compound spinal rod 1000. Referring first to FIG. 10A which shows the components of compound spinal rod 1000. As shown in FIG. 10A, compound spinal rod 1000 includes a first rod 1020 and a second rod 1040. Rod 1020 includes a ball-shaped retainer 1022 at one end (similar in design to retainer 202 of FIG. 2A) and a coupling 1024 at the other end—in this case merely the cylindrical surface of the rod 1020 to which a conventional pedicle screw can be mounted. Retainer 1022 is preferably made of cobalt chrome. Rod 1020 is preferably made in one piece including coupling 1024 and retainer 1022. Rod 1040 has a housing 1030 at one end and a coupling 1044 at the other end. Rod 1040 is preferably made in one piece including coupling 1044 and housing 1030. Compound spinal rod 1000 also includes a cap 1010 having a bore therethrough 1012 and a sleeve 1050 having a bore therethrough 1052.

Compound spinal rod 1000 includes a compliant bushing 1006. Bushing 1006 has a round central aperture 1007 for receiving the rod 1020 (see also FIG. 10B). The bushing 1006 is made of a hard-wearing compliant polymer. Bushing 1006 is a compliant member which exerts a centering force upon rod 1020 to keep it in alignment with rod 1040. Bushing 1006 is preferably made from polycarbonate urethane (for example, Bionate®) or another hydrophilic polymer. The bushing 1006 can act as a fluid lubricated bearing and allow the rod 1020 to rotate about the longitudinal axis of the rod 1020. Compound spinal rod 1000 also includes a metal sleeve 1050. Sleeve 1050 has a central aperture for receiving bushing 1006. Sleeve 1050 has at its distal end a flange 1054 for securing retainer 1022 or rod 1020 into cavity 1032 of housing 1030.

Housing 1030 has a cavity 1032 oriented along the axis of rod 1040 and configured to receive retainer 1022, sleeve 1050, bushing 1006, and cap 1010. Cap 1010, in this embodiment, is designed to hold bushing 1006 in position around rod 1020 as well as secure sleeve 1050 within cavity 1032 of housing 1030. Bushing 1006 fits within sleeve 1050 with the aperture 1007 aligned with the central bore 1012 of cap 1010 (see FIG. 10B). Cap 1010 has sockets 1011 which are adapted to be engaged by a pin wrench for tightening cap 1010 to housing 1030. Cap 1010 is threaded in order to engage the threaded proximal end of cavity 1032. Cap 1010 is, in alternative embodiments, joined to housing 1030 by, for example, laser welding.

Referring now to FIG. 10B, which shows a sectional view of compound spinal rod 1000 as assembled. When assembled, Bushing 1006 is positioned within sleeve 1050. Rod 1020 is then positioned through aperture 1007 of bushing 1006. Cap 1010 is then pushed over coupling 1024 with coupling 1024 passing out of central bore 1012 of cap 1010. Sleeve 1050, retainer 1022 and bushing 1006 are pushed into cavity 1032 of housing 1030. Cap 1010 is then secured into the threaded proximal end of cavity 1032 of housing 1030.

The flange 1054 of sleeve 1050 secures ball-shaped retainer 1022 within a hemispherical pocket 1034 at the distal end of cavity 1032 while allowing rotation of ball-shaped retainer 1022. Sleeve 1050 thus secures retainer 1022 within housing 1030 and holds bushing 1006 around rod 1020. Cap 1010 secures both bushing 1006 and sleeve 1050 in position. Housing 1030, sleeve 1050, retainer 1022 and bushing 1006 form a linkage 1004 connecting rod 1020 and rod 1040 such that coupling 1024 of rod 1020 can move relative to coupling 1044 of rod 1040.Bushing 1006 is sized and configured such that bushing 1006 is compressed by deflection of rod 1020 towards sleeve 1050 in any direction.

Referring now to FIG. 10C which shows a perspective view of compound spinal rod 1000 as assembled. Rod 1040 is held in compliant alignment with rod 1020 by bushing 2006 but can pivot a few degrees in any direction as shown by arrows 1057 by compression of bushing 1006. Note that there is a gap 1053 between rod 1020 and cap 1010 which permits deflection of rod 1020 through a predefined range before deflection is limited by contact with cap 1010. Rod 1020 may also rotate 360 degrees about its long axis relative to rod 1040 as shown by arrow 1055. In this embodiment, the rod 1020 pivots and rotates about axes which pass through the center of retainer 1022. Compound spinal rod 1000, by incorporating linkage 1004, allows controlled and constrained motion between rod 1020 and rod 1040 thereby allowing for greater range of motion in a dynamic stabilization prosthesis and also reducing stresses on the dynamic stabilization prosthesis and the bones to which it is attached.

FIG. 10D shows an enlarged perspective view of bushing 1006. Bushing 1006 is made of a compliant material which permits movement of rod 1020 relative to shield 1050 (see FIG. 10A). The bushing 1006 effectively controls the deflection of the rod 1020 relative to rod 1040. Bushing 1006 is preferably made of a compliant biocompatible polymer, for example PCU or PEEK. The properties of the material and dimensions of bushing 1006 are selected to achieve the desired force/deflection characteristics for linkage 1004 (see FIG. 10C). In a preferred embodiment, the bushing is made of PCU, is 2 mm thick when uncompressed and may be compressed to about 1 mm in thickness by deflection of the rod 1020 before rod 1020 contacts cap 1010.

As can be seen from FIG. 10D, a relief 1005 forms a conical depression in the proximal surface of bushing 1006 surrounding the central aperture 1007 which receives rod 1020 (not shown). The removal of material from the proximal surface of bushing 1006 forms a relief 1005 adapted to allow compression of bushing 1006 without bushing 1006 becoming trapped/pinched between rod 1020 and collar 1010 (see FIG. 10B). Bushing 1006 may also be shaped to modify the compliance of bushing 1006, for example by providing additional regions of relief or voids within the body of bushing 1006.

FIG. 10E shows a perspective view of an alternative bushing 1006e, also having a relief 1005e in the proximal surface surrounding the central aperture 1007e which receives rod 1020. The relief 1005e is curved—the curve extending from the perimeter of central aperture 1007e to the proximal end of bushing 1006e which is engaged by collar 1010 upon assembly (see FIG. 10B). In this embodiment, the outer circumference of bushing 1006e is provided with a plurality of scallops 1009e. Scallops 1009e reduce the volume of material at the proximal end of bushing 1006e. Scallops 1009e serve to make the bushing 1006e more compliant/flexible. During deflection of rod 1020 (see FIG. 10C) the bushing 1006e can expand into the void left by scallops 1009e further reducing the possibility that bushing 1006e will become trapped between rod 1020 and collar 1010. The scallops are larger in depth at the proximal end of bushing 1006e (top in FIG. 10E) and taper towards this distal end of bushing 1006e (bottom in FIG. 10E). In the bushing 1006e, the scallops make the proximal end of bushing 1006e more compliant than the distal end of bushing 1006e. This is advantageous as the geometry of linkage 1004 results in greater compression at the proximal end of bushing 1006e than the distal end of bushing 1006e. Increasing the flexibility of the proximal end of bushing 1006e thus serves to balance out the forces applied to rod 1040 by the proximal and distal regions of bushing 1006e allowing for a more even distribution of loading and “work” within the bushing 1006e and improving the longevity of bushing 1006e.

FIG. 10F shows a perspective view of another alternative bushing 1006d. Bushing 1006d has a relief 1005f in the proximal surface surrounding the central aperture 1007f. Relief 1005f takes the form of a conical depression in the proximal surface of bushing 1006f. Bushing 1006f also has a plurality of voids 1009f which penetrate from the proximal surface of bushing 1006f into the body of bushing 1006f along an axis parallel to the axis of central aperture 1007d. As shown in FIG. 10F, voids 1009f are circular in section. Voids 1009f may be, for example cylindrical apertures which pass all the way through bushing 1006f. Alternatively, the voids 1000f may be cylindrical apertures which pass part of the way but not all of the way through bushing 1006f. Alternatively, voids 1009f may be conical voids in which the size of the void diminishes as the void passes through bushing 1006f. The voids serve similar functions as scallops 1009e of FIG. 10E. For example, voids 1009f serve to increase the compliance of the material/region of bushing 1009f and provide space for the bushing to be pushed into by rod 1040 thereby avoiding pinching between rod 1040 and collar 1010 (See FIG. 10B).

FIG. 10G shows a sectional view of another alternative bushing 1006g. As shown in FIG. 10G, bushing 1006g includes a plurality of voids 1009g within the body of bushing 1006g. Voids 1006g spiral out from a position adjacent central aperture 1007g towards the outer edge of bushing 1006g. As shown, voids 1009g may be larger towards the outer edge of bushing 1006g where there is more material. As previously discussed voids 1009g may have a different cross-section at different levels in bushing 1006g. For example, voids 1009g may have a larger area at the proximal end of bushing 1006g (closest to collar 1010 of FIG. 10B) than at the distal end of bushing (closest to retainer 1022 of FIG. 10B) thereby increasing the flexibility of bushing 1006g where rod 1020 has the greatest amount of deflection. The voids 1009g serve similar functions as scallops 1009e of FIG. 10E. For example, the voids 1009g serve to increase the compliance of the material/region of bushing 1006g and provide space for the bushing 1006g to be pushed into by rod 1020 thereby avoiding pinching between rod 1020 and collar 1010 (See FIG. 10B).

The bushings 1006, 1006c, 1006d and 1006e show alternative configurations designed to achieve the function of controlling the movement of a rod within a linkage. Such bushings may be incorporated into any of the deflection rod systems described herein. Different designs and combinations of relief and voids than those illustrated may be utilized to adjust the flexibility of the bushing and prevent pinching of the bushing between the rod and other components of the linkage.

Compound spinal rod 1000 can be utilized in the prostheses, linkages, and assemblies as described above and illustrated for example in FIGS. 1D, 1E, 2E, 4C, 4D, 5E, 6C and 6D and accompanying text. Compound spinal rod can be modified through the use of different couplings on the rods including rods, apertures, ball-joints, pivoting joints and the like as shown for example in FIGS. 8A and 9A-9C.

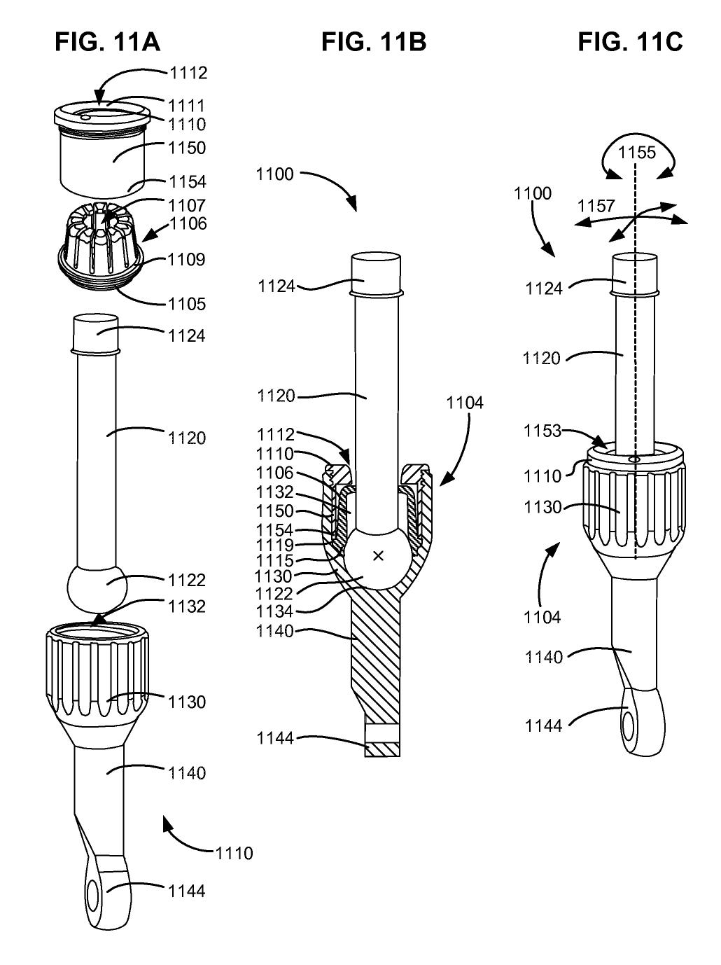

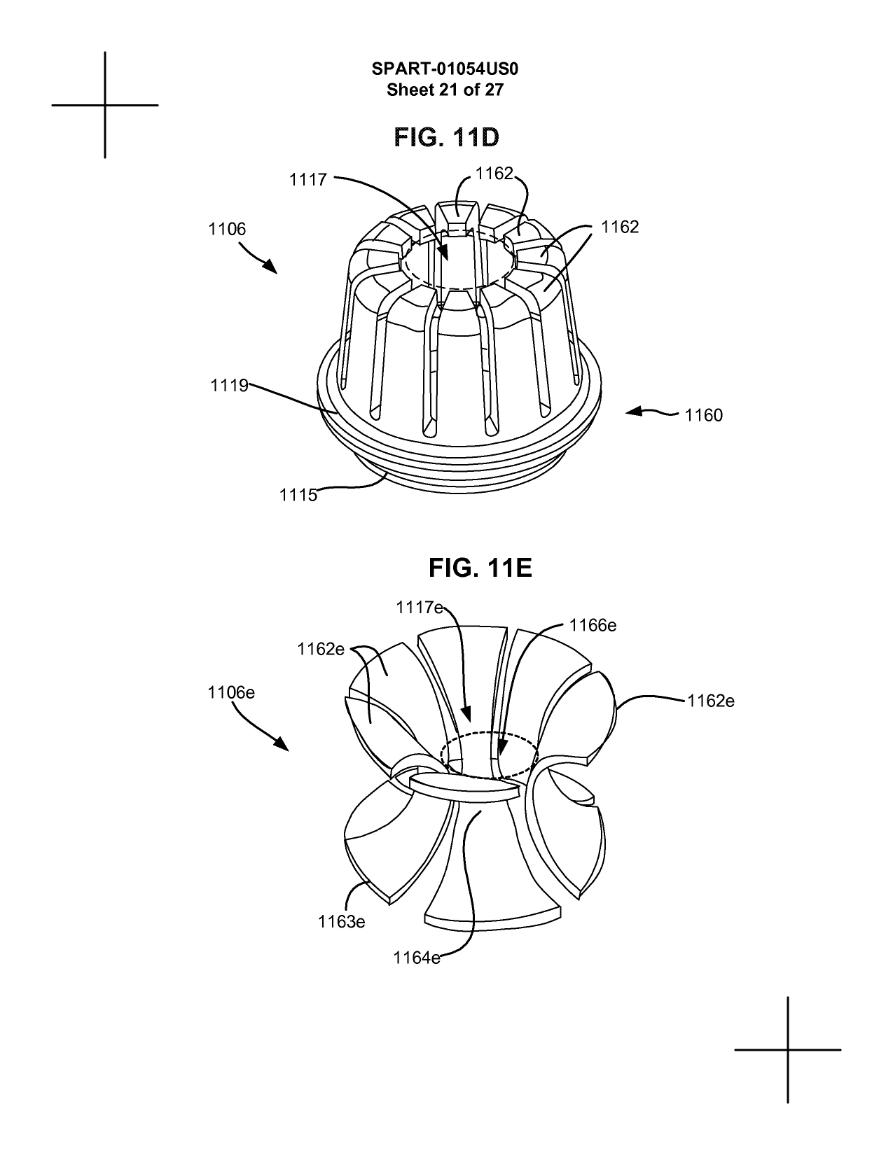

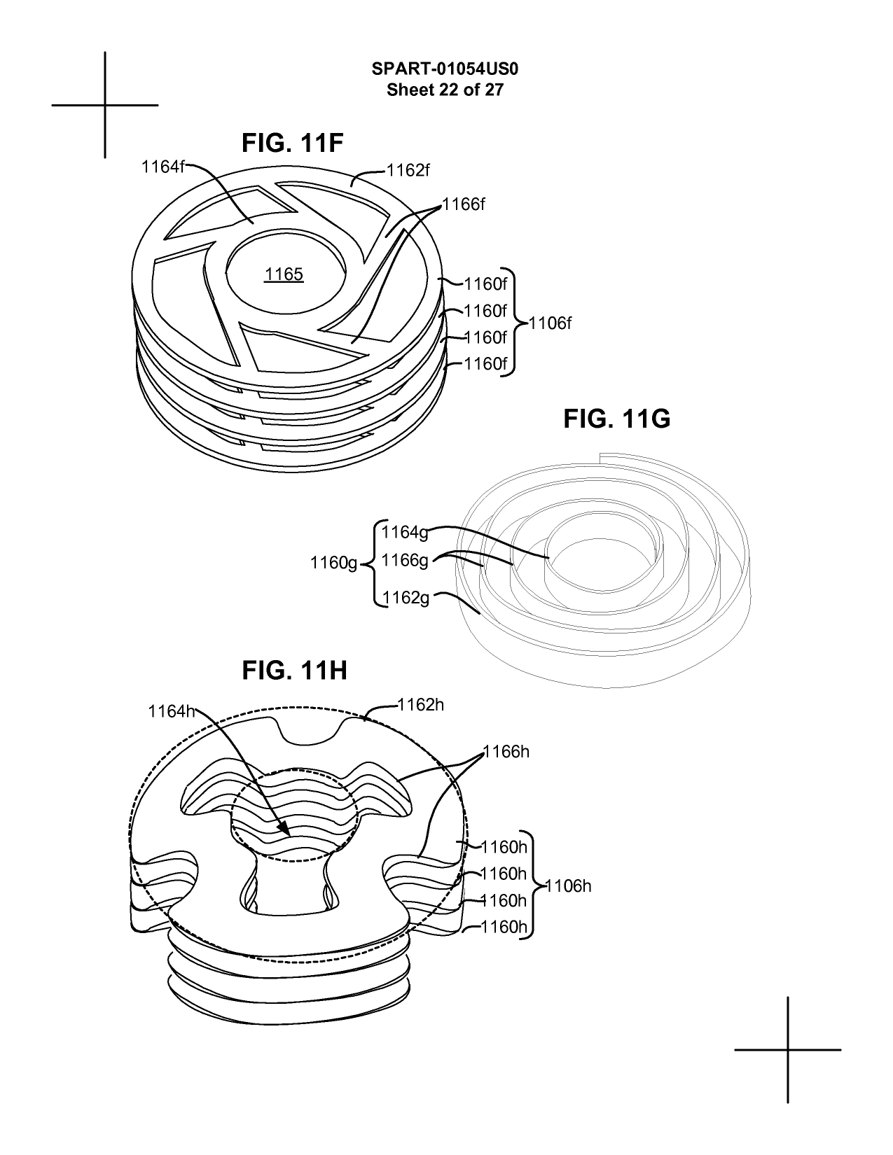

FIGS. 11A, 11B, and 11C are exploded, sectional, and perspective views of an alternative compound spinal rod according to an embodiment of the present invention. FIG. 11D shows an enlarged perspective view of the compliant member of the compound spinal rod of FIGS. 10A-10C. FIGS. 11E-11H show views of alternative compliant members for the compound spinal rod of FIGS. 11A-11C.

Referring first to FIG. 11A which shows the components of compound spinal rod 1100. As shown in FIG. 11A, compound spinal rod 1100 includes a first rod 1120 and a second rod 1140. Rod 1120 includes a ball-shaped retainer 1122 at one end and a coupling 1124 at the other end—in this case merely the cylindrical surface of the rod 1120 to which a conventional pedicle screw can be mounted. Retainer 1122 is preferably made of cobalt chrome. Rod 1120 is preferably made in one piece including coupling 1124 and retainer 1122. Rod 1140 has a housing 1130 at one end and a coupling 1144 at the other end. Rod 1140 is preferably made in one piece including coupling 1144 and housing 1130.

Compound spinal rod 1100 includes a compliant centering spring 1106. Centering spring 1106 has a round central aperture 1107 for receiving the rod 1120 (see also FIG. 11B). The centering spring 1106 is made of a hard-wearing compliant polymer. Centering spring 1106 is a compliant member which exerts a centering force upon rod 1120 to keep it in alignment with rod 1140. Centering spring 1106 is preferably made from polyetheretherketone PEEK. Centering spring 1106 has an internal flange 1115 at the distal end for engaging the retainer 1122. Centering spring also has an external rim 1119 for engaging the lower edge 1154 of sleeve 1150.

Compound spinal rod 1100 also includes a cap 1110 having a bore therethrough 1112. Cap 1110 also includes an integrated sleeve 1150 through which bore 1112 passes. Bore 1112 is size to receive a portion of centering spring 1106. The lower edge 1154 of sleeve 1150 is adapted to engage the rim 1119 of centering spring 1106 to secure it within cavity 1132 of housing 1130. having a bore therethrough 1152. Sleeve 1150 has a central aperture for receiving. The distal end 1154 of sleeve 1150 is designed to engage rim 1119 of centering spring 1116 for securing centering spring 1116, and retainer 1122 into cavity 1132 of housing 1130.

Housing 1130 has a cavity 1132 oriented along the axis of rod 1140 and configured to receive retainer 1122, sleeve 1150, centering spring 1106, and cap 1110. Cap 1110, in this embodiment, is designed to hold centering spring 1106 in position around rod 1120 as well as secure sleeve 1150 within cavity 1132 of housing 1130. Centering spring 1106 fits partially within sleeve 1150 with the aperture 1107 aligned with the central bore 1112 of cap 1110 (see FIG. 11B). Cap 1110 has sockets 1111 which are adapted to be engaged by a pin wrench for tightening cap 1110 to housing 1130. Cap 1110 is threaded in order to engage the threaded proximal end of cavity 1132. Cap 1110 is, in alternative embodiments, joined to housing 1130 by, for example, laser welding.

Referring now to FIG. 11B, which shows a sectional view of compound spinal rod 1100 as assembled. When assembled, Centering spring 1106 is partially positioned within sleeve 1150. The distal end 1154 of sleeve 1150 engages rim 1119 of centering spring 1116. Rod 1120 is positioned through aperture 1107 of centering spring 1106, through aperture 1112 of cap 1110 and sleeve 1150. Sleeve 1150, retainer 1122 and centering spring 1106 are pushed into cavity 1132 of housing 1130. Cap 1110 is then secured into the threaded proximal end of cavity 1132 of housing 1130.

The flange 1115 of sleeve 1106 secures ball-shaped retainer 1122 within a hemispherical pocket 1134 at the distal end of cavity 1132 while allowing rotation of ball-shaped retainer 1122. The distal end 1154 or sleeve 1150 secures centering spring 1106 against retainer 1122 within housing 1130 and holds centering spring 1106 around rod 1120. Cap 1110 secures centering spring 1106, retainer 1122 and sleeve 1150 in position. Housing 1130, sleeve 1150, retainer 1122 and centering spring 1106 form a linkage 1104 connecting rod 1120 and rod 1140 such that coupling 1124 of rod 1120 can move relative to coupling 1144 of rod 1140. Centering spring 1106 is sized and configured such that centering spring 1106 is compressed by deflection of rod 1120 towards sleeve 1150 in any direction.

Referring now to FIG. 11C which shows a perspective view of compound spinal rod 1100 as assembled. Rod 1140 is held in compliant alignment with rod 1120 by centering spring 1106 but can pivot a few degrees in any direction as shown by arrows 1157 by deforming centering spring 1106. Note that there is a gap 1153 between rod 1120 and cap 1110 which permits deflection of rod 1120 through a predefined range before deflection is limited by contact with cap 1110. Rod 1120 may also rotate 360 degrees about its long axis relative to rod 1140 as shown by arrow 1155. In this embodiment, the rod 1120 pivots and rotates about axes which pass through the center of retainer 1122. Compound spinal rod 1100, by incorporating linkage 1104, allows controlled and constrained motion between rod 1120 and rod 1140 thereby allowing for greater range of motion in a dynamic stabilization prosthesis and also reducing stresses on the dynamic stabilization prosthesis and the bones to which it is attached.

FIG. 11D shows an enlarged view of centering spring 1106. As shown in FIG. 11D, centering spring 1106 comprises a ring-shaped base 1160 from which extends a plurality of lever arms 1162. The lever arms extend upwards from base 1160 and extend in towards the central axis of ring-shaped base 1160. The lever arms 1162 define an aperture 1117 which is large enough for the passage of rod 1140 (not shown). Ring-shaped base 1160 also includes rim 1119 which is engaged by the distal end 1154 of the sleeve 1150 (See FIG. 11B).

The centering spring 1106 is selected such that the lever arms 1162 resist bending away from the position shown and thus resist deflection of rod 1140. The stiffness of compound spinal rod 1100 is affected by the spring rate of centering spring 1106. The stiffness of the compound spinal rod 1100 can be changed for example by increasing the spring rate of centering spring 1106 and conversely, the stiffness may be reduced by decreasing the spring rate of centering spring 1106. The spring rate of the centering spring 1106 can be, for example, increased by increasing the thickness of the lever arms 1162 and/or decreasing the length of the lever arms 1162. Alternatively and/or additionally changing the materials of the centering spring 1106 can also affect the spring rate. For example, making centering spring 1106 out of stiffer material increases the spring rate and thus reduces deflection of rod 1140 for the same amount of load—all other factors being equal. Centering spring 1106 is preferably made of a biocompatible polymer or metal. Centering spring 1106 may, for example, be made from PEEK, Bionate®, Nitinol, steel and/or titanium.

The stiffness of the compound spinal rod 1100 is also affected by factors beyond the spring rate of centering spring 1106. By changing the dimensions and or geometry of the rod 1140, centering spring 1106 and the sleeve 1150, the deflection characteristics of the compound spinal rod 1100 can be changed. For example, the stiffness of the compound spinal rod 1100 can be increased by increasing the distance from the pivot point of the rod 1140 to the point of contact between the lever arms 1162 surrounding aperture 1164 and the rod 1140. Conversely, the stiffness of the compound spinal rod 1100 can be decreased by decreasing the distance from the pivot point of the rod 1140 to the point of contact between the lever arms 1162 surrounding aperture 1164 and the rod 1140. The stiffness of the compound spinal rod may thus be varied or customized according to the needs of a patient by controlling the material and design of centering spring 1106 and other components of linkage 1104.

FIG. 11E shows an enlarged view of an alternative spring 1106e. As shown in FIG. 11E, spring 1106 comprises a plurality of spring elements 1162e. Each spring element 1162e is in the form of a leaf spring. Each spring element 1162e has a first end 1165e and a second end 1163e shaped to engage the sleeve 1150 of cap 1110 (see FIG. 11B) and maintain the orientation of the spring elements 1162e. Between the first end 1165e and second end 1163e, the spring elements curve in towards a raised middle section 1164e which is designed to engage the rod 1140 (see FIG. 11B). When the plurality of spring elements 1162e is assembled, the middle sections 1164 define an aperture 1166 sized to receive the rod 1140. When assembled with rod 1140, movement of rod 1140 pushes on middle section 1164 of one or more spring elements 1162e causing the one or more spring elements 1162e to flatten out. The spring elements resist this deformation and apply a restoring force to the rod 1140 to cause it to return to the center position. The force applied to rod 1140 is dependent upon the spring rate of spring elements 1162e and the amount of deflection of rod 1140.

Spring elements 1162e may be individual elements as shown, or they may be joined together, for example at the first ends 1165e and/or second ends 1163e. If joined together, spring elements 1162e may all be connected, or may be connected in two parts such that the two parts may be assembled from either side of rod 1140 during assembly with sleeve 1150. Spring elements 1162e may, in some embodiments, be formed in one piece, for example, machined or molded from a single block of material. In other embodiments, spring elements 1162e may be formed as separate pieces and then attached to one another.

The spring rate of each spring element 1162e may be controlled during design by choice of the design, dimensions and material of the spring element 1162e. For example, making the material of the spring elements 1162e thicker or reducing the length of the spring element 1162e can increase the spring rate of the spring element. Also, the material of the spring element 1162e may be selected to achieve the desired force-deflection characteristics. The spring elements 1162e may be identical thereby resulting in a force-deflection curve that is substantially uniform in all directions (isotropic). In other embodiments, the spring elements may have different spring rates thereby allowing the force-deflection curve of the deflection rod to be anisotropic—i.e. the deflection of rod 1140 has different force-deflection characteristics in different directions. Spring elements 1162e are in embodiments made from biocompatible metals (e.g.) titanium; superelastic metals (e.g.) titanium and/or biocompatible polymers (e.g. PEEK).

The spring/spring elements in the compound spinal rod of FIGS. 11A-11E are designed to elastically deform in the radial direction (relative to rod 1104). In alternative embodiments, different spring designs are used to control deflection of rod 1104 including, for example, spring washers, Belleville washers/disc springs, CloverDome™ spring washers, CloverSprings™, conical washers, wave washers, coil springs and finger washers. For example, a centering spring can includes one or more planar planer spring elements. Each planar spring element can be cut or stamped from a flat sheet of material. The planar spring elements are preferably made of a biocompatible elastic polymer or metal. For example, the planar spring elements may be made from, Bionate®, Peek, Nitinol, steel and/or titanium. The dimensions and material of the planar spring elements and rod are selected to achieve the desired force-deflection characteristics for deflectable the rod. In some embodiments, the number of planar spring elements used in a particular compound spinal rod may be selectable such that stiffer compound spinal rods have a larger number of planar spring elements and more compliant deflection rods have a lower number of planar spring elements. In other embodiments, the spring rate of each planar spring element may be adjusted by design, dimension or material changes.

FIG. 11F shows an enlarged view of one possible embodiment of a centering spring 1106f which includes a plurality of planar spring elements 1160f. As shown in FIG. 11F, planar spring element 1160f comprises an inner ring 1164f connected to an outer ring 1162f by a plurality of oblique lever arms 1166f. Outer ring 1162f is sized to fit within the cavity 1132 of housing 1130 (See FIG. 11A). Inner ring 1164f is sized so that aperture 1165f just fits over rod 1104. The arrangement of lever arms 1166f allows inner ring 1164f to deflect laterally with respect to outer ring 1162f by deforming lever arms 1166f. The lever arms 1166f resist the deformation. When assembled with rod 1104 and housing 1130 inner ring 1164f engages rod 1104 and outer ring 1162f engages housing 1130. When rod 1104 deflects towards housing 1130, lever arms 1166f are elastically deformed. The planar spring elements 1160f impart a return force upon rod 1104, pushing it away from housing 1130 toward the center (neutral position). The force applied by spring 1106f to rod 1104 is dependent upon the spring rate of planar spring elements 1160f and the amount of deflection of rod 1104.

FIG. 11G shows an enlarged view of an alternative embodiment of a spring element 1160g. As shown in FIG. 11G, spring element 1160g is a coil spring. The coil spring 1160g is wound to form an inner ring 1164g and an outer ring 1162g. The outer ring 1162g is sized to fit within cavity 1132 (See FIG. 11B). The inner ring 1164g is sized so that aperture 1165g just fits over rod 1104. Between inner ring 1164g and outer ring 1162g, are a plurality of helical coils 1166g. The arrangement of coils 1166g allows inner ring 1164g to deflect laterally with respect to outer ring 1162g by deforming coils 1166g. The coils 1166g resist the deformation. When assembled with rod 1104 and housing 1130, coil spring 1160g imparts a return force upon rod 1104 when rod 1104 deflects towards housing 1130 (see FIG. 11B). One or more coil springs 1160g may be used in the compound spinal rod of FIGS. 11A-11C.

FIG. 11H shows an enlarged view of an alternative embodiment of a spring 1106h comprising a plurality of domed spring washers 1160h. The domed spring washer 1160h has an inner aperture 1164h and an outer circumference 1162h. The outer circumference 1162h is sized to fit within cavity 1132 (see FIG. 11B). The inner aperture 1164h is sized to fit over rod 1104. Domed spring washer 1160h has a plurality of interior and exterior cutouts 1166h. These cutouts 1166h increase the compliance of domed spring washer 1160h (but reduce stiffness). The cutouts are designed to allow the desired degree of lateral deformation while still providing the desired spring rate. The pattern of cutouts 1166h shown in FIG. 11H forms a clover pattern but other patterns may be used, for example, fingers. The design of domed spring washer 1160h allows inner aperture 1164h to deflect laterally with respect to outer circumference 1162h by deforming the material of domed spring washers 1160h. The material of domed spring washers 1160h resists the deformation. When assembled with rod 1104 and housing 1130 of FIG. 11B, domed spring washers 1160h of spring 1106h impart a return force upon rod 1104 when rod 1104 deflects towards housing 1130. One or more spring washers 1160h may be used in the deflection rod of FIGS. 11A-11C.

Compound spinal rod 1100 can be utilized in the prostheses, linkages, and assemblies as described above and illustrated for example in FIGS. 1D, 1E, 2E, 4C, 4D, 5E, 6C and 6D and accompanying text. Compound spinal rod can be modified through the use of different couplings on the rods including rods, apertures, ball-joints pivoting joints and the like as shown for example in FIGS. 8A and 9A-9C.

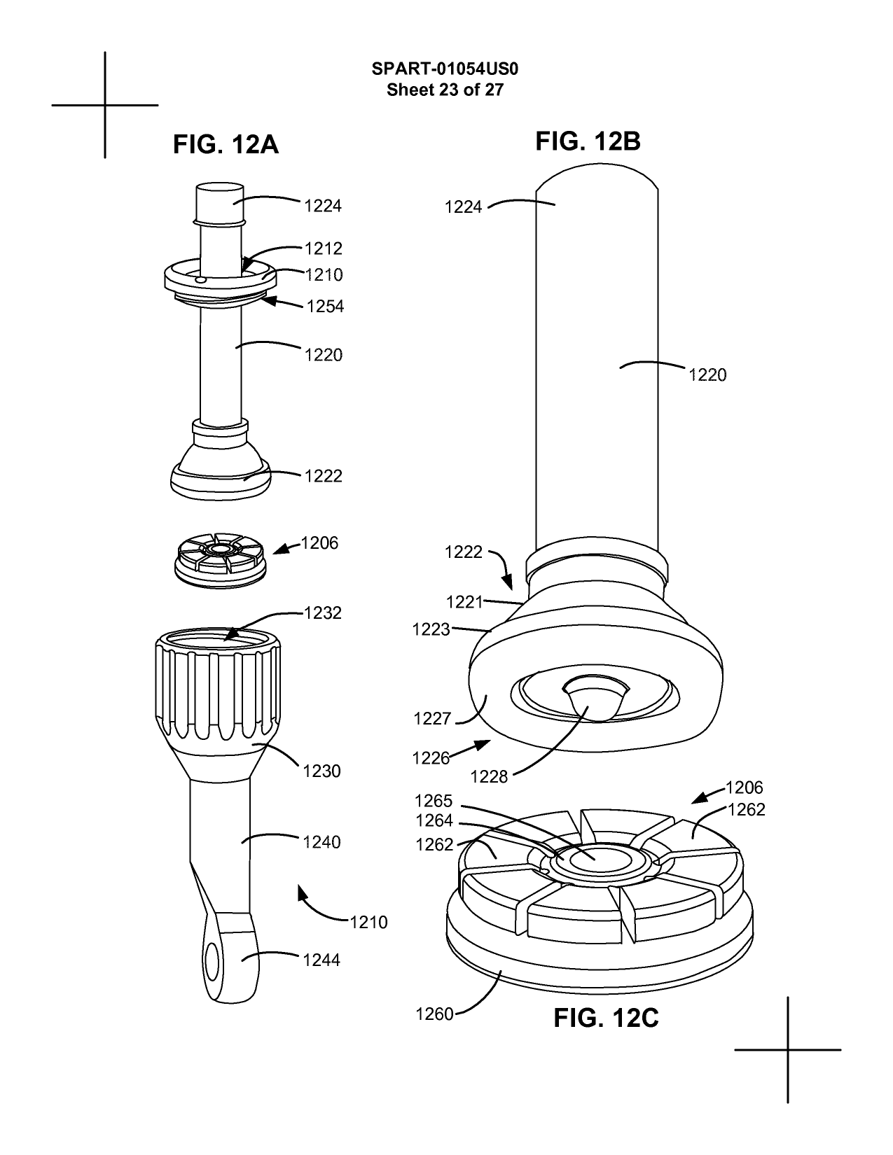

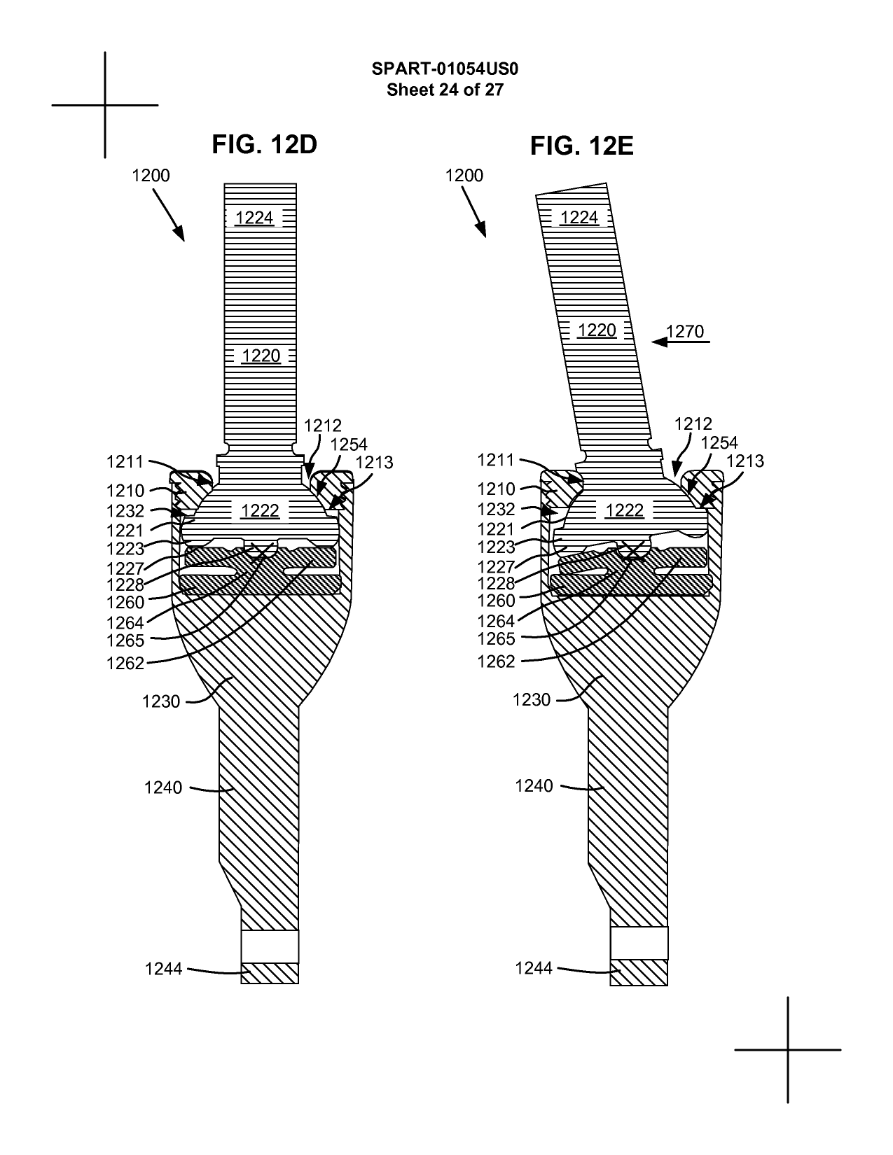

FIGS. 12A through 12E illustrate the design and operation of another embodiment of a compound spinal rod according to the present invention. FIG. 12A shows an exploded view of compound spinal rod 1200. As shown in FIG. 12A, compound spinal rod 1200 includes a first rod 1220 and a second rod 1240, a spring 1206, and a cap 1210. Rod 1220 includes generally hemispherical retainer 1222 at one end and a coupling 1224 at the other end—in this case merely the cylindrical surface of the rod 1220 to which a conventional pedicle screw can be mounted. Retainer 1222 is preferably made of cobalt chrome. Rod 1220 is preferably made in one piece including coupling 1224 and retainer 1222. Rod 1240 has a housing 1230 at one end and a coupling 1244 at the other end. Rod 1240 is preferably made in one piece including coupling 1244 and housing 1230. Housing 1230 has a cavity 1232 oriented along the axis of rod 1240 and configured to receive spring 1206 and retainer 1222.

Centering spring 1206 is a compliant member which exerts a centering force upon retainer 1222 to keep rod 1220 in alignment with rod 1240 (See, e.g., FIGS. 12D, 12E). Centering spring 1206 fits within cavity 1232 between retainer 1222 and the end of cavity 1232. Centering spring 1206 is in this embodiment, axially compressible. To put it another way, deflection of rod 1220 away from alignment with the axis of rod 1240 compresses spring 1206 in a direction generally parallel to the axis of rod 1240. Centering spring 1206 is preferably made from polyetheretherketone PEEK.

Compound spinal rod 1200 also includes a cap 1210 having a bore therethrough 1212. Cap 1210 is designed to hold retainer 1222 in cavity 1232 of housing 1230. Bore 1212 is sized to fit rod 1220 so that rod 1220 can extend through bore 1212 out of cavity 1232. The lower edge 1254 of cap 1210 is adapted to engage the retainer 1222 to secure it within cavity 1232 of housing 1230. Cap 1210 is threaded in order to engage the threaded proximal end of cavity 1232. Cap 1210 is, in alternative embodiments, joined to housing 1130 by, for example, laser welding.

FIG. 12B shows an enlarged perspective view of rod 1220, retainer 1222 and coupling 1224, which are made in one piece in this embodiment. Coupling 1224 is formed at the proximal end of rod 1220. In this case, coupling 1224 is merely the cylindrical surface of the rod 1220 to which a conventional pedicle screw can be mounted. Retainer 1222 can be made of cobalt chrome. Rod 1220 is preferably made in one piece including coupling 1224 and retainer 1222. In alternative embodiments, retainer 1222 and/or mount 1224 may be formed separately from rod 1220 and attached to rod 1220 by laser welding, soldering or other bonding technology. Alternatively, retainer 1222 and/or mount 1224 may mechanically engage the rod 1220.

Retainer 1222 has a curved proximal surface 1221 which is generally hemispherical. Rod 1220 extends from the center of curved proximal surface 1221. At the edge of curved proximal surface 1221 is a lip 1223. The distal surface 1226 is generally planar and oriented perpendicular to the longitudinal axis of rod 1220. The distal surface 1226 has a peripheral ridge 1227 adjacent the periphery for deflecting the spring 1206. The distal surface 1226 also has a central nub 1228 which forms the pivot point about which rod 1220 may deflect.

FIG. 12C shows an enlarged perspective view of spring 1206. As shown in FIG. 12C, spring 1206 comprises a circular base 1260. From the middle of circular base 1260 protrudes a column 1264 having a curved indentation 1265 at the proximal end for receiving nub 1228 of rod 1220. Extending laterally from column 1264 is a plurality of lever arms 1262. The material of spring 1206 is selected such that the lever arms resist bending away from the position shown. Circular base 1260 is designed to mate to the distal end of cavity 1232 to hold spring 1206 with lever arms 1262 held perpendicular to the longitudinal axis of bone anchor 1224 in the unloaded state.

The stiffness of compound spinal rod 1200 is affected by the spring rate of spring 1206. The stiffness of the compound spinal rod 1200 can be changed, for example, by increasing the spring rate of spring 1206 and conversely the stiffness may be reduced by decreasing the spring rate of spring 1206. The spring rate of spring 1206 can be increased by increasing the thickness of the lever arms 1262 and/or decreasing the length of the lever arms 1262. Alternatively and/or additionally changing the materials of the spring 1206 can also affect the spring rate. For example, making spring 1206 out of stiffer material increases the spring rate and thus reduces deflection of deflectable rod 1220 for the same amount of load—all other factors being equal. Spring 1206 is preferably made of a biocompatible polymer or metal. Spring 1206 may, for example, be made from PEEK, Bionate®, Nitinol, steel and/or titanium.

Spring 1206 may have the same spring rate in each direction of deflection of the rod 1220 (isotropic). The spring 1206 may have different spring rates in different directions of deflection of the rod 1220(anisotropic). For example, the spring 1206 can be designed to have different spring rate in different directions by adjusting, for example, the length, thickness and/or material of the lever arms 1262 in one direction compared to another direction. A compound spinal rod 1200 incorporating an anisotropic spring would have different force-deflection characteristics imparted to it by the spring 1206 in different directions.

The stiffness of the compound spinal rod 1200 is also affected by factors beyond the spring rate of spring 1206. By changing the dimensions and or geometry of rod 1220, spring 1206 and the retainer 1222, the deflection characteristics of the compound spinal rod 1200 can be changed. For example, the stiffness of the compound spinal rod 1200 can be increased by increasing the distance from the pivot point of the rod 1220 to the point of contact between the lever arms 1262 and the retainer 1222. The stiffness of the compound spinal rod may thus be varied or customized according to the needs of a patient

Referring now to FIGS. 12D and 12E, which show sectional views of a fully assembled compound spinal rod 1200. When assembled, spring 1206 is positioned in the distal end of cavity 1232 of housing 1230. Retainer 1222 is inserted into cavity 1230 so that nub 1228 of retainer 1202 engages indentation 1265 of spring 1206. Ridge 1226 of retainer 1202 makes contact with lever arms 1262. Collar 1210 is positioned over rod 1220 and secured into the threaded opening of cavity 1232. Collar 1232 has a curved surface 1212 which is complementary to the curved surface 1240 of retainer 1202. Collar 1210 secures retainer 1202 within cavity 1230 and traps spring 1206 between retainer 1202 and housing 1230.

When assembled, rod 1220 may pivot about the center of rotation defined by spherical surface 1240—marked by an “X” in FIG. 12E. Rod 1220 may also rotate about its longitudinal axis. FIG. 12E shows a partial sectional view of a fully assembled compound spinal rod 1200. As shown in FIG. 12E, spring 1206 occupies the space between retainer 1202 and housing 1230. When rod 1220 deflects from a position coaxial with bone anchor 1220, ridge 1226 pushes on spring 1206 compressing spring 1206. The spring 1206 is compressed in a direction parallel to the axis of rod 1240. To put it another way a load applied transverse to the axis of the rod 1220 as shown by arrow 1270 is absorbed by compression of spring 1206 in a direction generally parallel to the axis of bone anchor 1220 as shown by arrow 1272.

FIG. 12E illustrates deflection of rod 1220 from alignment with rod 1240. Applying a transverse load to rod 1220 as shown by arrow 1270 causes deflection of rod 1220 relative to shield 1208. Initially rod 1220 pivots about a pivot point 1203 indicated by an X. In this embodiment, pivot point 1203 is located at the center of ball-shaped retainer 1202. In other embodiments, however, pivot point 1203 may be positioned at a different location. For example, for other retainer shapes disclosed in the applications incorporated by reference herein, the retainer may pivot about a point which is at the edge of the retainer or even external to the retainer. As shown in FIG. 12E, deflection of rod 1220 deforms the spring 1206. The force required to deflect rod 1220 from alignment with rod 1240 depends upon the dimensions of rod 1220, spring 1206 and shield 1208 as well as the attributes of the material of spring 1206. In particular, the spring rate of spring 1206 and elements thereof (See FIG. 12B) may be adjusted to impart the desired force-deflection characteristics to compound spinal rod 1200.

As shown in FIG. 12E, after further deflection, rod 1220 comes into contact with limit surface 1211 of collar 1210. Limit surface 1211 is oriented such that when rod 1220 makes contact with limit surface 1211, the contact is distributed over an area to reduce stress on rod 1220 and limit surface 1211. Lip 1242 of retainer 1202 is positioned so that it makes simultaneous contact with the lower limit surface 1213 of collar 1210 on the opposite side of collar 1210. As depicted, the limit surface 1211 is configured such that as the rod 1220 deflects into contact with the limit surface 1211, the limit surface 1211 is aligned/flat relative to the rod 1220 in order to present a larger surface to absorb any load an also to reduce stress or damage on the deflectable.

Additional deflection of rod 1220 after contact with limit surface 1211 may cause elastic deformation (bending) of rod 1220. Because rod 1220 is relatively stiff, the force required to deflect rod 1220 increases significantly after contact of rod 1220 with the limit surfaces 1211, 1213 of collar 1210. For example, the stiffness may double upon contact of the rod 1220 with the limit surfaces 1211, 1213 of collar 1210. In a preferred embodiment, the proximal end of rod 1220 may deflect from 0.5 mm to 12 mm before rod 1220 makes contact with limit surfaces 1211, 1213. More preferably rod 1220 may deflect approximately 1 mm before making contact with limit surfaces 1211, 1213.

Thus as load or force is first applied to the compound spinal rod 1200 by the spine, the deflection of the compound spinal rod responds about linearly to the increase in the load during the phase when deflection of rod 1220 causes compression of spring 1206 as shown in FIG. 12E. After about 1 mm of deflection, when rod 1220 contacts limit surface 1211 and lip 1242 contacts lower limit surface 1213 (as shown in FIG. 12E) the compound spinal rod becomes stiffer. Thereafter a greater amount of load or force needs to be placed on the compound spinal rod in order to obtain the same incremental amount of deflection that was realized prior to this point because further deflection requires bending of rod 1220. Accordingly, the compound spinal rod 1200 provides a range of motion where the load supported increases about linearly as the deflection increases and then with increased deflection the load supported increases more rapidly in order to provide stabilization. To put it another way, the compound spinal rod 1200 becomes stiffer or less compliant as the deflection/load increases.

Compound spinal rod 1200 can be utilized in the prostheses, linkages, and assemblies as described above and illustrated, for example, in FIGS. 1D, 1E, 2E, 4C, 4D, 5E, 6C and 6D and accompanying text. Compound spinal rod can be modified through the use of different couplings on the rods including rods, apertures, ball-joints pivoting joints and the like as shown for example in FIGS. 8A and 9A-9C.

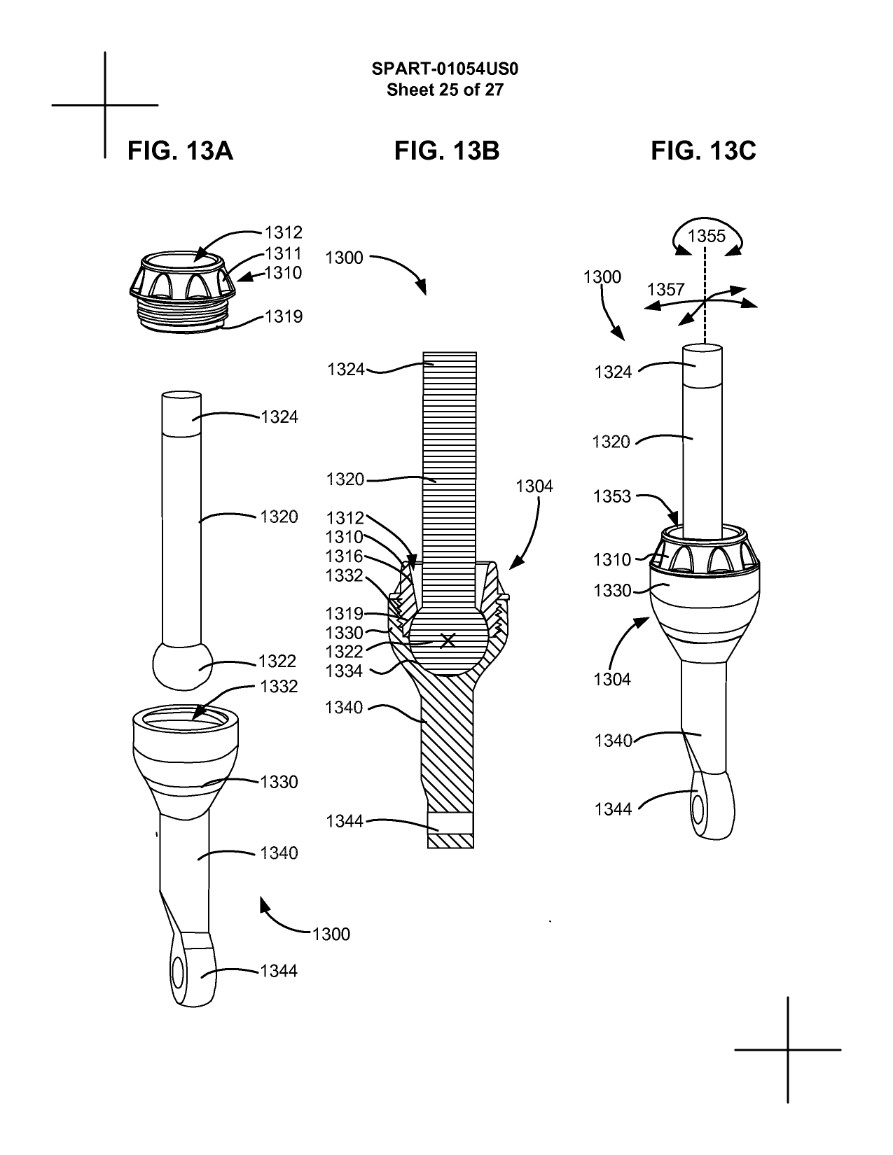

FIGS. 13A, 13B, and 13C are exploded, sectional, and perspective views of an alternative compound spinal rod according to an embodiment of the present invention. Referring first to FIG. 13A which shows the components of compound spinal rod 1300. As shown in FIG. 13A, compound spinal rod 1300 includes a first rod 1320 and a second rod 1340.

Rod 1320 includes a ball-shaped retainer 1322 at one end (similar in design to retainer 202 of FIG. 2A) and a coupling 1324 at the other end—in this case merely the cylindrical surface of the rod 1320 to which a conventional pedicle screw can be mounted. Retainer 1322 is preferably made of cobalt chrome. Rod 1320 is preferably made in one piece including coupling 1324 and retainer 1322.

Rod 1340 has a housing 1330 at one end and a coupling 1344 at the other end. Rod 1340 is preferably made in one piece including coupling 1344 and housing 1330. Housing 1330 has a cavity 1332 oriented along the axis of rod 1340 and configured to receive retainer 1322 and cap 1310.

Compound spinal rod 1300 also includes a cap 1310 having a bore therethrough 1312. Cap 1310, in this embodiment, is designed to secure retainer 1322 within housing 1330 and limit the range of motion of rod 1320. Cap 1310 has surface features 1311 which are adapted to be engaged by a wrench for tightening cap 1310 to housing 1330. Cap 1310 is threaded in order to engage the threaded proximal end of cavity 1332. Cap 1310 is, in alternative embodiments, joined to housing 1330 using other fastening features and or bonding technology, for example, laser welding.

Referring now to FIG. 13B, which shows a sectional view of compound spinal rod 1300 as assembled. Rod 1320 is positioned through central bore 1312 of cap 1310. Cap 1310 is then secured into the threaded proximal end of cavity 1332 of housing 1330. A flange 1319 of cap 1310 secures ball-shaped retainer 1322 within a hemispherical pocket 1334 at the distal end of cavity 1332 while allowing rotation of ball-shaped retainer 1322. Cap 1310 secures retainer 1322 within housing 1330 while allowing rotation and pivoting of first rod 1320 relative to second rod 1340. Housing 1330, retainer 1322 and cap 1310 form a linkage 1304 connecting rod 1320 and rod 1340 such that coupling 1324 of rod 1320 can move relative to coupling 1344 of rod 1340. A conical surface 1316 of bore 1312 operates as a limit surface to limit the angle through which rod 1320 may pivot relative to rod 1340.

Referring now to FIG. 13C which shows a perspective view of compound spinal rod 1300 as assembled. Rod 1340 can pivot a few degrees in any direction as shown by arrows 1357. Note that there is a gap 1353 between rod 1320 and cap 1310 which permits deflection of rod 1320 through a predefined range before deflection is limited by contact with cap 1310. Rod 1320 may also rotate 360 degrees about its long axis relative to rod 1340 as shown by arrow 1355. In this embodiment, the rod 1320 pivots and rotates about axes which pass through the center of retainer 1322. Compound spinal rod 1300, by incorporating linkage 1304, allows constrained motion between rod 1320 and rod 1340 thereby allowing for greater range of motion in a dynamic stabilization prosthesis and also reducing stresses on the dynamic stabilization prosthesis and the bones to which it is attached.

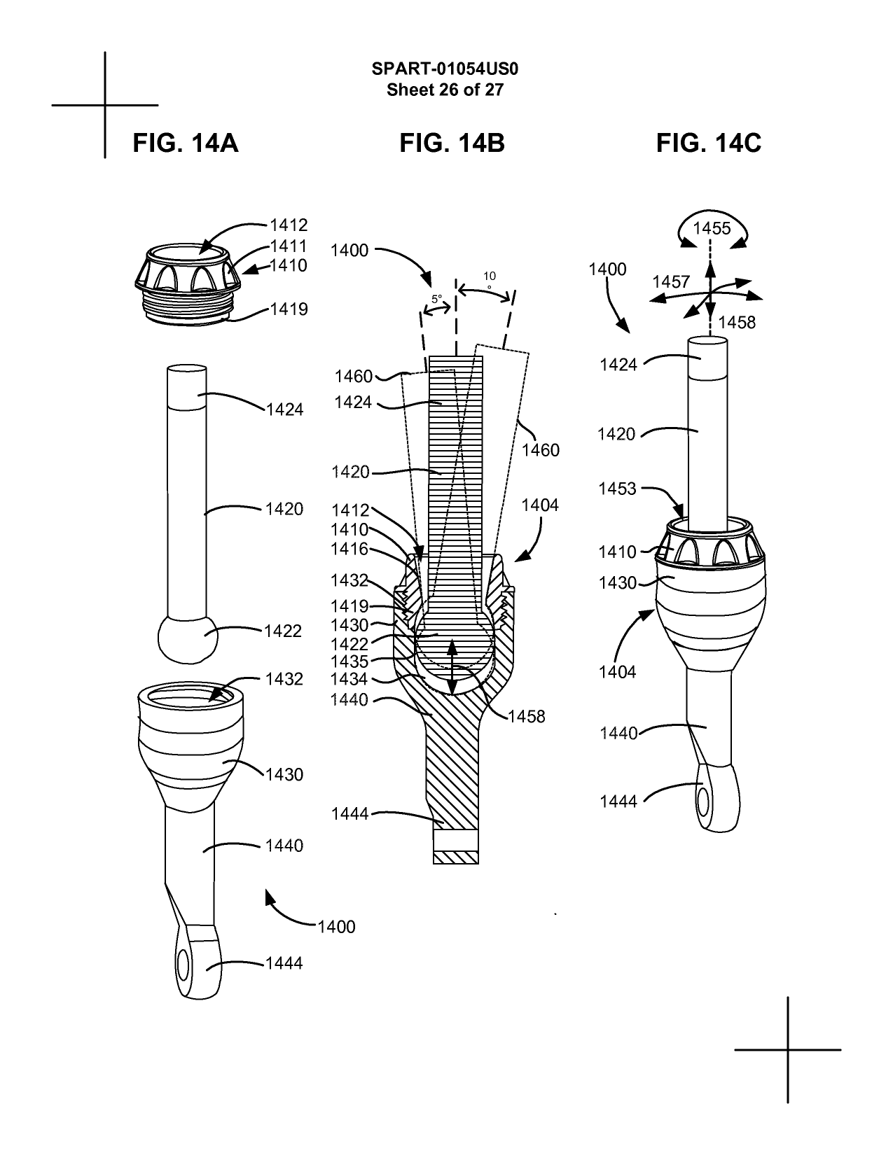

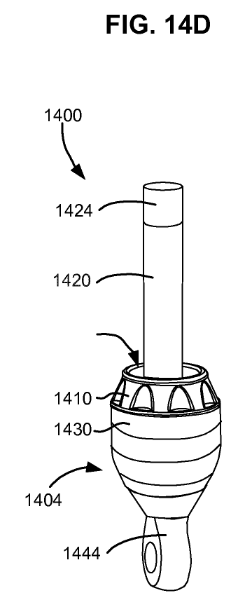

FIGS. 14A, 14B, and 14C are exploded, sectional, and perspective views of an alternative compound spinal rod according to an embodiment of the present invention. Referring first to FIG. 14A which shows the components of compound spinal rod 1400. As shown in FIG. 14A, compound spinal rod 1400 includes a first rod 1420 and a second rod 1440.

Rod 1420 includes a ball-shaped retainer 1422 at one end (similar in design to retainer 202 of FIG. 2A) and a coupling 1424 at the other end—in this case merely the cylindrical surface of the rod 1420 to which a conventional pedicle screw can be mounted. Retainer 1422 is preferably made of cobalt chrome. Rod 1420 is preferably made in one piece including coupling 1424 and retainer 1422.

Rod 1440 has a housing 1430 at one end and a coupling 1444 at the other end. Rod 1440 is preferably made in one piece including coupling 1444 and housing 1430. Housing 1430 has a cavity 1432 oriented along the axis of rod 1440 and configured to receive retainer 1422 and cap 1410.

Compound spinal rod 1400 also includes a cap 1410 having a bore therethrough 1412. Cap 1410, in this embodiment, is designed to secure retainer 1422 within housing 1430 and limit the range of motion of rod 1420. Cap 1410 has surface features 1411 which are adapted to be engaged by a wrench for tightening cap 1410 to housing 1430. Cap 1410 is threaded in order to engage the threaded proximal end of cavity 1432. Cap 1410 is, in alternative embodiments, joined to housing 1430 using other fastening features and or bonding technology, for example, laser welding.

Referring now to FIG. 14B, which shows a sectional view of compound spinal rod 1400 as assembled. Rod 1420 is positioned through central bore 1412 of cap 1410. Cap 1410 is then secured into the threaded proximal end of cavity 1432 of housing 1430. Cap 1410 secures retainer 1422 within housing 1430 while allowing rotation and pivoting of first rod 1420 relative to second rod 1440. A flange 1419 of cap 1410 secures ball-shaped retainer 1422 within a hemispherical pocket 1434 at the distal end of cavity 1432.

In the embodiment of FIGS. 14A-14C, cavity 1432 includes a cylindrical extension 1435 in addition to hemispherical pocket 1434. Retainer 1422 is free to slide within cylindrical extension 1435 until limited by hemispherical pocket 1434 or flange 1419. Thus rod 1420 can slide towards and away from rod 1440 as shown by arrow 1458. The range of sliding motion is selected based upon the range of movement desired between adjacent vertebrae and can be from between 1 mm and 10 mm, but is more preferably between 1 mm and 5 mm, for example 2 mm.

As with the embodiment of FIGS. 13A-13C, retainer 1422 of FIGS. 14A-14C is free to rotate within cavity 1432 thus allowing rod 1420 to pivot and rotate relative to rod 1440. The range through which rod 1420 can pivot is limited by contact between rod 1420 and cap 1410 and in particular the conical interior surface 1416 within bore 1412. In preferred embodiments the angular range of motion is constrained to be within 1 and 10 degrees from axial alignment with rod 1540. It should be noted however that the range through which rod 1420 can pivot increases as retainer 1422 moves towards cap 1410 and away from the base of hemispherical pocket 1434. Thus, in the example shown in FIG. 13B, the range of pivoting motion of rod 1420 is constrained to 5 degrees from alignment with rod 1440 when retainer 1422 is in contact with hemispherical pocket 1434 (see outline 1460). However, the range of pivoting motion of rod 1420 is constrained to 10 degrees from alignment with rod 1440 when retainer 1422 is in contact with flange 1419 (see outline 1462).

Housing 1430, retainer 1422 and cap 1410 form a linkage 1404 connecting rod 1420 and rod 1440 such that coupling 1424 of rod 1420 can move relative to coupling 1444 of rod 1440. A conical surface 1416 of bore 1412 operates as a limit surface to limit the angle through which rod 1420 may pivot relative to rod 1440.

Referring now to FIG. 14C which shows a perspective view of compound spinal rod 1400 as assembled. Rod 1440 can pivot a few degrees in any direction as shown by arrows 1457. Note that there is a gap 1453 between rod 1420 and cap 1410 which permits deflection of rod 1420 through a predefined range before deflection is limited by contact with cap 1410. Rod 1420 may also rotate 360 degrees about its long axis relative to rod 1440 as shown by arrow 1455. In this embodiment, the rod 1420 pivots and rotates about axes which pass through the center of retainer 1422. Compound spinal rod 1400, by incorporating linkage 1404, allows constrained motion between rod 1420 and rod 1440 thereby allowing for greater range of motion in a dynamic stabilization prosthesis and also reducing stresses on the dynamic stabilization prosthesis and the bones to which it is attached.

FIG. 14D is a perspective view of a variation of the compound spinal rod of FIGS. 14A-14C according to an embodiment of the present invention. In the variation shown in FIGS. 14D, second rod 1440 includes coupling 1444. The length of the rods in this and other embodiments is selected such that the compound sliding rod is sized for spanning from one vertebra to an adjacent vertebra. Thus, in embodiments, the rods are from 10 to 50 mm in length. The embodiment of FIG. 14D illustrates a variation in which the length of the second rod 1440 is small. As shown in FIG. 14D, the length of second rod 1440 is such that second rod 1444 is entirely coupling 1444 and there is no shaft intervening between coupling 1444 and housing 1430. A similar configuration may also be applied to each of the embodiments of compound vertical rods described above such that the coupling of the second rod is essentially directly connected to the housing of the second rod and preferably formed in one piece with the housing of the second rod.

### Materials for Embodiments of the Invention

As desired, the implant can, in part, be made of titanium, titanium alloy, or stainless steel. The balls and other components that have surface moving relative to another surface are, in some embodiments, made of coated with cobalt chrome. In some cases Nitinol or nickel-titanium (NiTi) or other super elastic materials including copper-zinc-aluminum and copper-aluminum-nickel are used for elements of the implant, however for biocompatibility, nickel-titanium is the preferred material. The compliant members including: o-rings, bushings and the like are formed of complaint polymers or metals. In systems where a deflectable post or rod will rotate relative to the compliant member, the compliant member is preferably made of a hydrophilic polymer which can act as a fluid lubricated bearing. A preferred material for making the compliant members is a polycarbonate urethane including, for example Bionate®. Bionate® is available in a variety of grades which are selected based upon the design of the implant and the force/deflection attributes desired or necessary for the application. Another preferred material for making the compliant members is polyetheretherketone (PEEK).

Other suitable materials include, for example: polyetherketoneketone (PEKK), polyetherketone (PEK), polyetherketone-etherketoneketone (PEKEKK), and polyetherether-ketoneketone (PEEKK), and polycarbonate urethane (PCU). Still, more specifically, the material can be PEEK 550G, which is an unfilled PEEK approved for medical implantation available from Victrex of Lancashire, Great Britain. (Victrex is located at www.matweb.com or see Boedeker www.boedeker.com). Other sources of this material include Gharda located in Panoli, India (www.ghardapolymers.com). Reference to appropriate polymers that can be used in the spacer can be made to the following documents. These documents include: PCT Publication WO 02/02158 A1, dated Jan. 10, 2002, entitled “Bio-Compatible Polymeric Materials;” PCT Publication WO 02/00275 A1, dated Jan. 3, 2002, entitled “Bio-Compatible Polymeric Materials;” and PCT Publication WO 02/00270 A1, dated Jan. 3, 2002, entitled “Bio-Compatible Polymeric Materials.”

As will be appreciated by those of skill in the art, other suitable similarly biocompatible thermoplastic or thermoplastic polycondensate materials that resist fatigue, have good memory, are flexible, and/or deflectable have very low moisture absorption, and good wear and/or abrasion resistance, can be used without departing from the scope of the invention.

The foregoing description of preferred embodiments of the present invention has been provided for the purposes of illustration and description. It is not intended to be exhaustive or to limit the invention to the precise forms disclosed. Many embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the claims and their equivalents.

What is claimed is:

1. A compound spinal rod comprising:
a first rod,
the first rod having a housing at one end and a first coupling at the other end;
a bore in the housing aligned with a longitudinal axis of the first rod, the bore having an open end and a closed end and the closed end of the bore terminating in a hemispherical pocket;
a second rod;
a ball-shaped retainer at one end of the second rod and a second coupling at the other end of the second rod,
wherein the second rod is received in the bore in the housing of the first rod such that the ball-shaped retainer is positioned within the hemispherical pocket and the coupling of the second rod extends through the open end of the bore;
a fastener which secures the ball-shaped retainer in the hemispherical pocket such that the ball-shaped retainer may pivot and rotate within the hemispherical pocket; and
a compliant member positioned within the bore between the second rod and the housing such that deflection of the second rod away from alignment with the first rod causes compression of the compliant sleeve such that the compliant sleeve applies a force upon the second rod pushing the second rod towards a position in which the second rod is aligned with the first rod.

2. The compound spinal rod of claim 1, wherein:
said housing is associated with a limit surface positioned to contact the second rod after a predetermined amount of deflection of the second rod away from alignment with the first rod; and
wherein further deflection of said second rod beyond said predetermined amount of deflection requires a larger load per unit of deflection than deflection of said second rod up to said predetermined amount of deflection.

3. The compound spinal rod of claim 1, wherein:
said housing is associated with a limit surface positioned to contact the second rod after a predetermined amount of deflection of the second rod away from alignment with the first rod; and
wherein further deflection of said second rod beyond said predetermined amount requires at least double the load per unit of deflection than deflection of said second rod up to said predetermined amount of deflection.

4. The compound spinal rod of claim 1, wherein:
said second rod and ball-shaped retainer are made in one piece; and
said housing and first rod are made in one piece.

5. The compound spinal rod of claim 1, wherein said compliant member is a polymer o-ring.

6. The compound spinal rod of claim 1, wherein said compliant member is a hydrophilic polymer.

7. The compound spinal rod of claim 1, wherein said ball-shaped retainer comprises cobalt chrome.

8. The compound spinal rod of claim 1, wherein said fastener comprises:
a central bore adapted to receive the second rod;
a first end adapted to fit within the bore of the second rod;
the first end having a curved surface adapted to secure the ball-shaped retainer in the hemispherical pocket such that the ball-shaped retainer may pivot and rotate within the hemispherical pocket; and
a second end which includes a limit surface positioned to contact the second rod after a predetermined amount of deflection of the second rod away from alignment with the first rod.

9. The compound spinal rod of claim 1, wherein the first coupling has an aperture adapted to mount to a post of a bone anchor.

10. The compound spinal rod of claim 1, wherein the housing has an exterior surface and wherein said exterior surface is fluted to be adapted to engage the exterior surface by one of a connector and a driver.

11. A compound spinal rod comprising:
a first rod having a first end and a second end;
a second rod having a first end and a second end;
a joint which secures the second end of the second rod to the first end of the first rod such that the second rod may pivot relative to the first rod;
a tubular extension of the first rod which extends over a portion of the second rod adjacent the joint; and
a compliant member disposed between the portion of the second rod adjacent the joint and the tubular extension of the first rod whereby the compliant member biases the second rod into alignment with the first rod.

12. The compound spinal rod of claim 11, further comprising:
a limit surface associated with the tubular extension and positioned to contact the second rod when the second rod pivots through a first angle from alignment with the first rod; and
wherein the limit surface resists pivoting of said second rod beyond said first angle.

13. The compound spinal rod of claim 11, wherein:
the second rod is aligned with a longitudinal axis of the first rod when unloaded; and
wherein application of a load on the first end of the second rod causes the second rod to pivot away from alignment with a longitudinal axis of the first rod thereby compressing the compliant member between the second rod and the tubular extension.

14. The compound spinal rod of claim 11, wherein:
said first rod and tubular extension are made in one piece.

15. The compound spinal rod of claim 11, wherein said joint comprises a ball-joint.

16. The compound spinal rod of claim 11, wherein said compliant member is a polymer disc having an outer diameter sized to fit with the tubular extension and a central aperture sized to receive the post.

17. The compound spinal rod of claim 11, wherein said joint is adapted to permit the second rod to rotate around a longitudinal axis of the second rod

18. A spinal implant comprising:
an elongate rod having a first end and a second end;
an elongate post having a first end and a second end;
a joint which secures the second end of the post to the first end of the rod such that the post can pivot relative to the rod;
a tubular cap having a bore, the tubular cap extending over a distal portion of the post, the tubular cap having a fastener which secures the tubular cap to the first end of the rod; and
a compliant ring disposed between the post and the tubular cap whereby the compliant ring biases the post into alignment with the rod.

19. The spinal implant of claim 18, wherein:
the bore has a circumferential groove therein; and
the compliant ring is retained in said circumferential groove.

20. The spinal implant of claim 18, wherein said tubular cap comprises a limit surface positioned to contact the post after a predetermined amount of deflection of the post away from alignment with the rod.

21. A compound spinal rod comprising:
a first rod,
the first rod having a housing at one end and a first coupling at the other end;
a bore in the housing aligned with a longitudinal axis of the first rod, the bore having an open end and a closed end and the closed end of the bore terminating in a hemispherical pocket;
a second rod;
a ball-shaped retainer at one end of the second rod and a second coupling at the other end of the second rod,
wherein the second rod is received in the bore in the housing of the first rod such that the ball-shaped retainer is positioned within the hemispherical pocket and the coupling of the second rod extends through the open end of the bore; and
a fastener which secures the ball-shaped retainer in the hemispherical pocket such that the ball-shaped retainer may pivot and rotate within the hemispherical pocket.

22. A compound spinal rod comprising:
a first rod having a first end and a second end;
a second rod having a first end and a second end;
a joint which secures the second end of the second rod to the first end of the first rod such that the second rod may pivot relative to the first rod; and
a tubular extension of the first rod which extends over a portion of the second rod adjacent the joint.

23. The compound spinal rod of claim 1 wherein the hemispherical pocket is elongated such that the ball-shaped retainer can move along a longitudinal axis of the second rod.

24. The compound spinal rod of claim 23 such that as the ball-shaped retainers move along the longitudinal axis of the second rod, the ability of the second rod to articulate relative to the first rod changes.
